(19) United States
(12) Patent Application Publication
(10) Pub. No.: US 2024/0156870 A1
(43) Pub. Date: May 16, 2024

(54) ANTI-EGFR SINGLE DOMAIN ANTIBODIES AND THERAPEUTIC CONSTRUCTS

(71) Applicant: National Research Council of Canada, Ottawa (CA)

(72) Inventors: Kevin HENRY, Ottawa (CA); Martin ROSSOTTI, Ottawa (CA); Mehdi ARBABI-GHAHROUDI, Ottawa (CA); Scott MCCOMB, Ottawa (CA); Risini WEERATNA, Ottawa (CA); Jamshid TANHA, Orleans (CA)

(21) Appl. No.: 18/550,715

(22) PCT Filed: Oct. 28, 2021
(86) PCT No.: PCT/CA2021/051525

§ 371 Date: Sep. 15, 2023

Related U.S. Application Data
(60) Provisional application No. 63/167,413, filed on Mar. 29, 2021.

(51) Int. Cl. A61K 35/17; A61K 39/00; A61P 35/00; C07K 16/28

(52) U.S. Cl. CPC A61K 35/17; A61K 39/4611; A61K 39/4631; A61K 39/464404; A61K 39/464411; A61P 35/00; C07K 16/2809; C07K 16/2863; A61K 2239/13; A61K 2239/29

(57) ABSTRACT

Herein are provided anti-EGFR single domain antibodies (sdAb) prepared by immunizing a llama with recombinant human EGFRvIII. VHH antibodies specific to EGFR were isolated. The example antibodies initially produced comprise CDR1, CDR2, and CDR3 sequences corresponding, respectively to SEQ NOs: 1-3, 4-6, 7-9, 10-12, 13-15, 16-18, 19-21, 22-24, 25-27, 28-30, 31-33, 34-36, 37-39, 40-42, 43-45, and 46-48; and related sequences, including humanized variants. Also provided are multivalent antibodies comprising any one of the sdAbs, including bispecific T-cell engagers, bispecific killer cell engagers (BiKEs), and trispecific killer cell engagers (TriKEs). Also described are chimeric antigen receptors (CARs) for CAR-T therapy comprising any one of the aforementioned sdAbs. Uses of these molecules in the treatment of cancer are also described, in particularly EGFR-high cancers. Hinge lengths may be selected to achieve desired activities, such as high activity or high selectivity for target vs. non-target cells.

### CROSS REFERENCE TO RELATED APPLICATIONS

This application claims the benefit of priority from U.S. Provisional Application No. 63/167,413 which is entitled “ANTI-EGFR SINGLE DOMAIN ANTIBODIES AND THERAPEUTIC CONSTRUCTS” and was filed Mar. 29, 2021, which is incorporated by reference in its entirety.

### FIELD

The present disclosure relates generally to anti-EGFR antibodies. More particularly, the present disclosure relates to ant-EGFR single domain antibodies.

### BACKGROUND

Cancer is a major public health problem and the second leading cause of death worldwide. Traditional therapy for cancer has included surgery, radiation and chemotherapy. These have been moderately successful for treatment of some cancers, particularly those diagnosed at early stages. However effective therapy is lacking for many aggressive cancers. Recent technological innovations suggest that immunotherapy (stimulating or restoring a patient's own immune system to fight cancer) can potentially provide potent and long term responses against many cancers including aggressive hard to treat cancers.

Immunotherapy has had phenomenal success in treating certain cancers. Antibodies have been used alone and in therapeutic constructs.

For example, bi-specific T cell engagers, and bi- and tri-specific killer cell engagers (BiKEs and TriKEs) incorporating single-chain variable fragments (scFvs) have been developed to direct a host's immune system to target cancer cell.

Chimeric Antigen Receptor (CAR) constructs have also been produced to combined facets of T cell activation into a single protein. These molecules link an extracellular antigen recognition domain to an intracellular signaling domain, which activates the T cell when an antigen is bound. Chimeric Antigen Receptor (CAR) modified immune cell therapies are an emergent form of cancer immunotherapy whereby single or multiple antigen binding domains from antibodies that specifically target cell surface protein(s) on cancer cells are combined with immune cell activating domains to generate “armored” Immune cells that seek and kill specific cells that harbor the targeting antigen(s). CAR modified T cell therapies (CAR-T) have provided unprecedented responses for patients suffering from certain aggressive forms of leukemia, for example.

Up-regulation of epidermal growth factor receptor (EGFR) is a hallmark of many solid tumors, and inhibition of EGFR signaling by small molecules and antibodies has clear clinical benefit. EGFR is a receptor tyrosine kinase that is overexpressed and constitutively activated in up to 80% of solid cancers [22]. Following the development of small molecule inhibitors, naked antibodies (Abs) against EGFR, exemplified by cetuximab, have shown clinical benefit in treating colorectal [23] and head and neck cancers [24]. Other EGFR Abs are under investigation in other indications and as Ab-drug conjugates. Camelid single-domain antibodies (sdAbs or VHHs) have previously been isolated against EGFR using protein immunization [25] and whole-cell immunization [26], and biparatopic molecules with improved potency have been constructed from these sdAb building blocks [27]. One advantage of sdAb-based biologics for cancer therapy is that they tend to penetrate solid tumors better than full-length IgGs [28].

It is, therefore, desirable to provide immunogenic molecule with affinity for EGFR.

### SUMMARY

It is an object of the present disclosure to obviate or mitigate at least one disadvantage of previous

In a first aspect, there is provided an isolated single domain antibody (sdAb), which binds specifically to human EGFR, the sdAb comprising:
a) a CDR1 amino acid sequence X₁X₂X₃X₄X₅X₆X₇X₈ (SEQ ID NO: 49), wherein:
X₁ is L or G,
X₂ is S, F, I, V, or F,
X₃ is D, A, I, or S,
X₄ is F or L,
X₅ is G, E, S, Y, or T,
X₆ is S, R, or I,
X₇ is K or N, and
X₈ is H, G, A, or T,
a CDR2 amino acid sequence X₉X₁₀X₁₁X₁₂GX₁₃T (SEQ ID NO: 50), wherein:
X₉ is I or V,
X₁₀ is F, S, T, or Y,
X₁₁ is S, L, R, or N,
X₁₂ is S, G, A, or is absent, and
X₁₃ is A, T, D, G, or S, and
a CDR3 amino acid sequence NX₁₄X₁₅PPX₁₆X₁₇X₁₈ (SEQ ID NO: 51), wherein:
X₁₄ is L, Y, or A,
X₁₅ is V or I,
X₁₆ is S, T, or F,
X₁ is P or S, and
X₁₈ is A, Y, or N,

b)
a CDR1 amino acid sequence
(SEQ ID NO: 22)
GSFFSRDA,
a CDR2 amino acid sequence
(SEQ ID NO: 23)
ISSGGLS,
and
a CDR3 amino acid sequence
(SEQ ID NO: 24)
SIYEDGVWTS,
c)
a CDR1 amino acid sequence
(SEQ ID NO: 25)
GSISSISV,
a CDR2 amino acid sequence
(SEQ ID NO: 26)
ITSGGIS,
a CDR3 amino acid sequence
(SEQ ID NO: 27)
YMYVHVGYWVDY,
d)
a CDR1 amino acid sequence
(SEQ ID NO: 28)
ESIDSIYV,
a CDR2 amino acid sequence
(SEQ ID NO: 29)
LTPGGGS,
a CDR3 amino acid sequence
SEQ ID NO: 30)
NAFHRLRGTHY,
or
e)
a CDR1 amino acid sequence
(SEQ ID NO: 31)
GFTFQEAY,
a CDR2 amino acid sequence
(SEQ ID NO: 32)
IDSSVGST,
a CDR3 amino acid sequence
(SEQ ID NO: 33)
GTYDTEETTSSWEDEYQYAV.

In another aspect, there is provided an isolated single domain antibody (sdAb), which binds specifically to human EGFR, the sdAb comprising:
A)
i) a CDR1 amino acid sequence as set forth in SEQ ID NO: 1, a CDR2 amino acid sequence as set forth in SEQ ID NO: 2, and a CDR3 amino acid sequence as set forth in SEQ ID NO: 3 (NRC-sdAb02l),
ii) a CDR1 amino acid sequence as set forth in SEQ ID NO: 4, a CDR2 amino acid sequence as set forth in SEQ ID NO: 5, and a CDR3 amino acid sequence as set forth in SEQ ID NO: 6 (NRC-sdAb022),
iii) a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9 (NRC-sdAb023),
iv) a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12 (NRC-sdAb024),
v) a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15 (NRC-sdAb025),
vi) a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18 (NRC-sdAb026),
vii) a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (NRC-sdAb027),
viii) a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (NRC-sdAb028),
ix) a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 (NRC-sdAb032),
x) a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30 (NRC-sdAb033), or
xi) a CDR1 amino acid sequence as set forth in SEQ ID NO: 31, a CDR2 amino acid sequence as set forth in SEQ ID NO: 32, and a CDR3 amino acid sequence as set fort in SEQ ID NO: 33 (NRC-VH54).

In another aspect, there is provided an isolated single domain antibody (sdAb), which binds specifically to human EGFR, the sdAb comprising:
A)
i) a CDR1 amino acid sequence as set forth in SEQ ID NO: 1, a CDR2 amino acid sequence as set forth in SEQ ID NO: 2, and a CDR3 amino acid sequence as set forth in SEQ ID NO: 3 (NRC-sdAb021),
ii) a CDR1 amino acid sequence as set forth in SEQ ID NO: 4, a CDR2 amino acid sequence as set forth in SEQ ID NO: 5, and a CDR3 amino acid sequence as set forth in SEQ ID NO: 6 (NRC-sdAb022),
iii) a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9 (NRC-sdAb023),
iv) a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12 (NRC-sdAb024),
v) a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15 (NRC-sdAb025),
vi) a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18 (NRC-sdAb026),
vii) a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (NRC-sdAb027),
viii) a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (NRC-sdAb028),
ix) a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 (NRC-sdAb032),
x) a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30 (NRC-sdAb033), or
xi) a CDR1 amino acid sequence as set forth in SEQ ID NO: 31, a CDR2 amino acid sequence as set forth in SEQ ID NO: 32, and a CDR3 amino acid sequence as set forth in SEQ ID NO: 33 (NRC-VH54); or
B)
CDR1, CDR2, and CDR3 amino acid sequences that are at least 80% identical to the CDR1, CDR2, and CDR3 sequences defined in any one of i) to xi).

In another aspect, there is provided an isolated VHH single domain antibody (sdAb), which binds specifically to human EGFR, the sdAb comprising:
A)
a CDR3 amino acid sequence as set forth in SEQ ID NO: 3,
a CDR3 amino acid sequence as set forth in SEQ ID NO: 6,
a CDR3 amino acid sequence as set forth in SEQ ID NO: 9,
a CDR3 amino acid sequence as set forth in SEQ ID NO: 12,
a CDR3 amino acid sequence as set forth in SEQ ID NO: 15,
a CDR3 amino acid sequence as set forth in SEQ ID NO: 18,
a CDR3 amino acid sequence as set forth in SEQ ID NO: 21,
a CDR3 amino acid sequence as set forth in SEQ ID NO: 24,
a CDR3 amino acid sequence as set forth in SEQ ID NO: 27,
a CDR3 amino acid sequence as set forth in SEQ ID NO: 30, or
a CDR3 amino acid sequence as set forth in SEQ ID NO: 33.

In one aspect, there is provided a recombinant polypeptide comprising an sdAb as defined herein.

In aspect, there is provided a recombinant nucleic acid molecule encoding an sdAb, the recombinant polypeptide, or the V_{H}H:F_{c} fusion as defined herein.

In one aspect, there is provided a composition comprising an sdAb as defined herein, or a polypeptide comprising such an sdAb; together with an acceptable excipient, diluent or carrier.

In one aspect, there is provided a use of the sdAb as defined herein or of an antibody comprising one or more V_{H}H:F_{c} fusion as defined herein for treatment of a cancer.

In one aspect, there is provided a use of the sdAb as defined herein or of an antibody comprising one or more V_{H}H:F_{c} fusion as defined herein for preparation of a medicament for treatment of a cancer.

In one aspect, there is provided the sdAb as defined herein or of an antibody comprising one or more V_{H}H:F_{c} fusion as defined herein for use in treatment of a cancer.

In one aspect, there is provided a method of treating a cancer in subject comprising administering to the subject the sdAb as defined herein or of an antibody comprising one or more V_{H}H:F_{c} fusion as defined herein.

In one aspect, there is provided a multivalent antibody comprising an sdAb as defined above.

In aspect, there is provided a recombinant nucleic acid molecule encoding the multivalent antibody as defined herein.

In one aspect, there is provided a composition comprising a multivalent antibody as defined herein; together with an acceptable excipient, diluent or carrier.

In one aspect, there is provided a use of the multivalent antibody as defined herein for treatment of a cancer.

In one aspect, there is provided a use of the multivalent antibody as defined herein for preparation of a medicament for treatment of a cancer.

In one aspect, there is provided the multivalent antibody as defined herein for use in treatment of a cancer.

In one aspect, there is provided a method of treating a cancer in subject comprising administering to the subject the multivalent antibody as defined herein.

In one aspect, there is provided a chimeric antibody receptor (CAR), which binds to human EGFR, comprising the VHH sdAb as defined herein.

In one aspect, there is provided a recombinant nucleic acid molecule encoding the CAR as defined herein.

In one aspect, there is provided a vector comprising the recombinant nucleic acid molecule as defined herein.

In one aspect, there is provided a recombinant viral particle comprising the recombinant nucleic acid as defined herein.

In one aspect, there is provided a cell comprising the recombinant nucleic acid molecule as defined herein.

In one aspect, there is providing a use of the nucleic acid, vector, or viral particle as described herein for preparation of cells for CAR-T.

In one aspect, there is providing a method of preparing cells for CAR-T comprising contacting a T-cell with the viral particle as described herein. In one embodiment, the T-cell is from a donor. In one embodiment, the T-cell is from a patient.

In one aspect, there is providing a method of preparing cells for CAR-T comprising introducing into a T-cell the nucleic acid or vector as described herein. In one embodiment, the T-cell is from a donor. In one embodiment, the T-cell is from a patient.

In one aspect, there is provided a use of the CAR or of the engineered cell as described herein for treatment of a cancer.

In one aspect, there is provided a use of the CAR or of the engineered cell as described herein for preparation of a medicament treatment of a cancer.

In one aspect, there is provided the CAR or the engineered cell as described herein for use in treatment of a cancer.

In one aspect there is provided a method of treating a cancer in a subject, comprising administering to the subject the engineered cell as defined herein.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 depicts results of serum titration ELISAs of the llama immunized against human EGFRvIII.

FIG. 2 depicts result of experiments showing binding of of VHHs to EGFR by SPR and ELISA. Panel A depicts SPR sensorgrams showing single-cycle kinetic analysis of VHH binding to human EGFR-Fc. Panel B provides a summary of epitope binning by SPR. Panel C depicts result of Competitive ELISA showing binding of VHHs to EGFR in the presence or absence of EGF.

FIG. 3A depicts binding of V_{H}H sdAb022 elicited by DNA immunization to human, rhesus and mouse EGFR-Fc in ELISA.

FIG. 3B depicts binding of V_{H}H sdAb028 elicited by DNA immunization to human, rhesus and mouse EGFR-Fc in ELISA.

FIG. 3C depicts binding of V_{H}H sdAb032 elicited by DNA immunization to human, rhesus and mouse EGFR-Fc in ELISA.

FIG. 3D depicts binding of V_{H}H sdAb033 elicited by DNA immunization to human, rhesus and mouse EGFR-Fc in ELISA.

FIG. 4A depicts sensorgrams for epitope binning SPR co-injection experiments.

FIG. 4B depicts sensorgrams for further epitope binning SPR co-injection experiments.

FIG. 4C depicts sensorgrams for further epitope binning SPR co-injection experiments.

FIG. 4D depicts sensorgrams for further epitope binning SPR co-injection experiments.

FIG. 4E depicts sensorgrams for further epitope binning SPR co-injection experiments.

FIG. 4F depicts sensorgrams for further epitope binning SPR co-injection experiments.

FIG. 4G depicts sensorgrams for further epitope binning SPR co-injection experiments.

FIG. 4H depicts sensorgrams for further epitope binning SPR co-injection experiments.

FIG. 4I depicts sensorgrams for further epitope binning SPR co-injection experiments.

FIG. 4J depicts sensorgrams for further epitope binning SPR co-injection experiments.

FIG. 4K depicts sensorgrams for further epitope binning SPR co-injection experiments.

FIG. 4L depicts sensorgrams for further epitope binning SPR co-injection experiments.

FIG. 4M depicts sensorgrams for further epitope binning SPR co-injection experiments.

FIG. 5A depicts binding of VHH monomers (20 μg/ml) to EGFR-positive MDA-MB-468 cells by flow cytometry detected using anti-c-Myc and AF647-labeled anti-mouse antibodies.

FIG. 5B depicts binding of serially diluted VHH-Fcs to EGFR-positive MDA-MB-468 cells by Mirrorball® microplate cytometry detected using AF488-labeled anti-human Fc antibody.

FIG. 5C depicts binding of serially diluted VHH-Fcs to EGFR-low MCF7 cells by Mirrorball® microplate cytometry detected using AF488-labeled anti-human Fc antibody.

FIG. 5D depicts binding of serially diluted biparatopic VHH-Fcs by Mirrorball® microplate cytometry detecting using AF488-labeled anti-human Fc antibody.

FIG. 6A depicts Western blotting of phosphorylated EGFR (Tyr1068), total EGFR and p-actin in serum-starved MDA-MB-468 cells treated with the indicated inhibitor (all at 500 nM) in the presence or absence of EGF.

FIG. 6B depicts densitometry analysis of bands in FIG. 6A.

FIG. 6C depicts Western blotting of phosphorylated EGFR (Tyr1068), total EGFR and p-actin in serum-starved MDA-MB-468 cells treated with the indicated inhibitor in the presence or absence of EGF.

FIG. 6D depicts densitometry analysis of bands in FIG. 6C. The concentrations used are as shown in FIG. 6C.

FIG. 6E depicts Western blotting of phosphorylated EGFR (Tyr1068), total EGFR and β-actin in serum-starved MDA-MB-468 cells treated with the indicated inhibitor (all at 5 nM) in the presence or absence of EGF.

FIG. 6F depicts densitometry analysis of bands in FIG. 6E.

FIG. 7, Panel A depicts structural elements of human EGFR and sdAb based EGFR-targeting CAR tested here. FIG. 7, Panels B, C, and D depicts results of experiments to assess EGFR-specific sdAbs with CAR functionality, in which Jurkat cells were electroporated with CAR constructs comprising EGFR targeted sdAb as antigen binding domain. Control cells with no plasmid or with a CD19-targeted scFv element were also tested here (FMC63-28z). Panel A depicts results for EGFR-high SKOV3. Panel B depicts results for EGFR-low MCF7 cells. Panel C depicts results for EGFR-negative Raji cells. Results show the mean+/−SEM from a single experiment performed in duplicate.

FIG. 8 depicts hinge truncations of EGFR-sdAb CAR constructs.

FIG. 9 depicts the result of experiments showing the effects of hinge truncations. Panel A to C depict results for Jurkat cells electroporated with varying CAR constructs before co-incubation with no target cells, or with EGFR-high SKOV3, or EGFR-low MCF7 as target cells at varying effector:target ratios as indicated on the X axis.

FIG. 10 depicts the results of experiments showing the relationship between hinge length and CAR response in EGFR-high and EGFR-low target cells. Panel A depicts data presented for CAR-J assay against EGFR-high SKOV3 cells as described in FIG. 9 reorganized to show the activation of EGFR sdCARs over a range of hinge lengths at an effector to target ratio of 1:10, varying from a full 45 amino acid human CD8 hinge to no hinge. Panel B depicts data from CAR-J data against EGFR-low MCF7 cells organized via hinge length. Panel C show the relative selectivity of hinge-modified EGFR sdCARs for SKOV3 over MCF7 is shown. Results depict the mean+/−SEM of a single experiment performed in duplicate.

FIG. 11 shows that CAR hinge sensitivity is highly dependent on target antigen and epitope location. Panel A A range of sdCAR constructs were produced containing the EGFR-specific sdAb021 and human CD8 hinge domains ranging between 1 and 45 residues in length. The full-length 45-residue CD8 hinge was extended with a Gly-Ser linker between 1 and 17 residues in length. Jurkat cells were electroporated with the resulting constructs and then co-incubated at an effector to target ratio of 1:1 with EGFR-high SKOV3 cells, EGFR-low MCF7 cells, or EGFR-negative NALM6 cells. After overnight incubation, CAR/GFP+ cells were examined for activation via staining with APC-labelled anti-human CD69 antibody. Panel B Similarly, trastuzumab-scFv HER2-specific CAR constructs were generated with hinge domains of varying lengths. CAR-expressing Jurkat cells were co-incubated with HER2-high SKOV3 cells, HER2-low MCF7 cells, or HER2-negative NALM6 cells and then CAR-T activation was assessed. Panel C Similarly, EGFRvIII-specific CAR constructs were generated with hinge domains of varying lengths. CAR-expressing Jurkat cells were co-incubated with EGFRvIII-overexpressing U87vIII cells or EGFRvIII-negative U87 wt cells and then CAR-T activation was assessed. Results show means+/−SEMs of three separate experiments.

FIG. 12 depicts the results of a high-throughput CAR activity assay in which Jurkat cells were transiently transfected with EGFR-CAR plasmids with varying hinge domains and subjected to a co-culture assay with or without EGFR-expressing target cells (SKOV3). Results demonstrate the ability to modulate CAR activity through modulating the hinge length and potential application in rationale design of CAR constructs.

FIG. 13 shows that sdCAR auto-activation shows no consistent association with hinge length. CAR-J activation data for the EGFR sdAb021 hinge-modified sdCARs with no target cells or with EGFR-negative NALM6 cells is shown. Results show the means+/−SEMs of three independent experiments.

FIG. 14 depicts red-fluorescent target cell counts for SKOV3 co-cultured with EGFR targeted CAR-T cells derived from three donors. Un-transduced donor T cells are used as negative controls.

FIG. 15 depicts red-fluorescent target cell counts MCF7 co-cultured for cells from three donors. with EGFR targeted CAR-T cells derived from three donors. Un-transduced donor T cells are used as negative controls.

FIG. 16 depicts fold changes in target cell expansion and CAR-T cell expansion at day 5 for summarized at varying effector:target ratios for donor 1 cells.

FIG. 17 depicts fold changes in target cell expansion and CAR-T cell expansion at day 5 for summarized at varying effector:target ratios for donor 2 cells.

FIG. 18 depicts the level of apparent surface EGFR expression on various target cell lines as determined using staining of single cell suspensions of various cell lines with a fluorescently labelled EGFR antibody and assessment via flow cytometry.

FIG. 19 depicts reduced killing of primary fibroblast cells with hinge domain truncation. Healthy donor T cells were transduced with lentiviral vectors (GFP marked) in order to induce expression of EGFR-targeted CAR molecules with varying hinge domains in co-culture with primary human fibroblast cells stably expressing a red-fluorescent protein.

FIG. 20 depicts relative growth of primary fibroblast cells (top) and CAR-T cells (bottom) in co-cultures experiments.

FIG. 21 shows that hinge-truncated sdCAR-T cells maintain selectivity for EGFR overexpressing cells following re-challenge. Donor blood derived T cells were transduced with lentiviral particles encoding the EGFR-specific sdAb021 sdCAR with varying hinge domains. The resulting sdCAR-T cells were challenged via co-culture with EGFR-high SKOV3 cells and examined for Panel A target cell expansion. Challenged cells were then diluted 1/10 with fresh media and challenged with (Panel B) EGFR-high SKOV3 cells, (Panel G) EGFR-medium U87vIII cells, or (Panel K) EGFR-low MCF7 cells. Similarly, cells challenged twice with SKOV3 cells were re-challenged with various target cells and examined for (Panels C, H, and L) target cell expansion. Each graph depicts automated cell counts or fluorescent areas from a single well from a single experiment.

FIG. 22 depicts sdCAR-T cell expansion data corresponding to FIG. 19. Panel D depicts sdCAR-T cell expansion data corresponding to FIG. 19, Panel A. Panel E depicts sdCAR-T cell expansion data corresponding to FIG. 19, Panel B. Panel I depicts sdCAR-T cell expansion data corresponding to FIG. 19, Panel G. Panel M depicts sdCAR-T cell expansion data corresponding to FIG. 19, Panel K. Panels F, J, and N depict sdCAR-T cell expansion data corresponding to FIG. 19, Panel C, H, and L, respectively. Each graph depicts automated cell counts or fluorescent areas from a single well from a single experiment.

FIG. 23 shows tumor volume estimated using caliper measurements for NOD/SCID/IL2γ-chain-null (NSG) mice injected subcutaneously with 2×10⁶ SKOV3 cells stably expressing mKate2.

FIG. 24 depicts time to humane endpoint (tumor volume 3000 mm³) for the experiments depicted in FIG. 21.

FIG. 25 depicts the final tumor measurement at day 112. The experiment was ended early due to COVID-19 related animal facility shutdown.

FIG. 26 depicts fluorescence imaging performed at varying time points after tumor cell injection.

FIG. 27 depicts proportion of human CD45+ cells that were GFP/CAR+n blood was collected at selected time points after tumor challenge to.

FIG. 28 depicts staining for human CCR7 and CD45RA was used to assess the differentiation status of CAR/GFP+ cells or total T cells for mice treated with mock-transduced CAR-T cells. The inset shows the gating strategy used to delineate effector, naïve, central memory, or effector memory-RA+ (EMRA) cells.

FIG. 29 depicts the molecular structure of EGFR-specific single domain antibody bi-specific T cell engager proteins; with an EGFR-sdAb sequence at the 5′ end of a DNA construct, followed by a linker sequence which can be of varying composition, herein depicted as either a flexible linker sequence or a flexible linker sequence followed by a human CD8-hinge domain, followed by a CD3-specific single chain variable fragment containing both a heavy chain and a light chain sequence.

FIG. 30 depicts higher killing of EGFR expressing U87vIII glioblastoma cells with a bi-specific T cell engager containing a human CD8-hinge spacer domain between EGFR-binding and CD3-binding domains. Cell supernatants from HEK293T cells transiently transfecting with plasmids expressing EGFR-CD3 bi-specific T cell engager molecules with or without CD8-hinge domain (shown in FIG. 29).

FIG. 31 depicts depicts relative growth of U87-vIII target cells in T-cell/tumour cell co-cultures with or without addition of cell supernatants containing various bi-specific T cell engager molecules.

### DETAILED DESCRIPTION

Generally, the present disclosure provides anti-EGFR single domain antibodies (sdAb) prepared by immunizing a llama with recombinant human EGFRvIII. VHH antibodies specific to EGFR were isolated. The example antibodies initially produced comprise CDR1, CDR2, and CDR3 sequences corresponding, respectively to SEQ NOs: 1-3, 4-6, 7-9, 10-12, 13-15, 16-18, 19-21, 22-24, 25-27, 28-30, 31-33, 34-36, 37-39, 40-42, 43-45, and 46-48; and related sequences, including humanized variants. Also provided are multivalent antibodies comprising any one of the sdAbs, including bispecific T-cell engagers, bispecific killer cell engagers (BiKEs), and trispecific killer cell engagers (TriKEs). Also described are chimeric antigen receptors (CARs) for CAR-T therapy comprising any one of the aforementioned sdAbs. Uses of these molecules in the treatment of cancer are also described, in particularly EGFR-high cancers. Hinge lengths may be selected to achieve desired activities, such as high activity or high selectivity for target vs. non-target cells.

Single Domain Antibodies & Polypeptides Comprising Them

A single domain antibody (sdAb), also known as a nanobody, is an antibody fragment consisting of a single monomeric variable antibody domain. sdAbs have been derived from heavy-chain antibodies found in Camelidae species (such as camel, llama, dromedary, alpaca and guanaco) using molecular biology techniques, which are also known as V_{H}H fragments (herein also termed “V_{H}H” or “VHH”). Other examples include V_{NAR}fragments derived from heavy chain antibodies found in cartilaginous fish, such as sharks. sdAbs have also been generated from a heavy chain/light chain of conventional immunoglobulin G (IgGs) by engineering techniques.

V_{H}H molecules are about 10 times smaller than IgG molecules. These single polypeptides are generally quite stable, often resisting extreme pH and temperature conditions that can be problematic for conventional antibodies and antibody fragments. Moreover, V_{H}Hs tend to be more resistant to the action of proteases. Furthermore, in vitro expression of V_{H}Hs tends to produce high yield of properly folded/functional VHHs. In addition, heavy chain antibodies and their engineered fragments (i.e., VHHs) generated in Camelidae species may recognize cryptic or hidden epitopes which otherwise inaccessible to larger conventional antibodies and antibody fragments generated in vitro through the use of antibody libraries or by immunization of other mammals.

In one aspect, there is provided an isolated single domain antibody (sdAb), which binds specifically to human EGFR, the sdAb comprising:
a) a CDR1 amino acid sequence X₁X₂X₃X₄X₅X₆X₇X₈ (SEQ ID NO: 49), wherein:
X₁ is L or G,
X₂ is S, F, I, V, or F,
X₃ is D, A, I, or S,
X₄ is F or L,
X₅ is G, E, S, Y, or T,
X₆ is S, R, or I,
X₇ is K or N, and
X₈ is H, G, A, or T,
a CDR2 amino acid sequence X₉X₁₀X₁₁X₁₂GX₁₃T (SEQ ID NO: 50), wherein:
X₉ is I or V,
X₁₀ is F, S, T, or Y,
X₁₁ is S, L, R, or N,
X₁₂ is S, G, A, or is absent, and
X₁₃ is A, T, D, G, or S, and
a CDR3 amino acid sequence NX₁₄X₁₅PPX₁₆X₁₇X₁₈ (SEQ ID NO: 51), wherein:
X₁₄ is L, Y, or A,
X₁₅ is V or I,
X₁₆ is S, T, or F,
X₁₇ is P or S, and
X₁₃ is A, Y, or N,

b)
a CDR1 amino acid sequence
(SEQ ID NO: 22)
GSFFSRDA,
a CDR2 amino acid sequence
(SEQ ID NO: 23)
ISSGGLS,
and
a CDR3 amino acid sequence
(SEQ ID NO: 24)
SIYEDGVWTS,
c)
a CDR1 amino acid sequence
(SEQ ID NO: 25)
GSISSISV,
a CDR2 amino acid sequence
(SEQ ID NO: 26)
ITSGGIS,
a CDR3 amino acid sequence
(SEQ ID NO: 27)
YMYVHVGYWVDY,
d)
a CDR1 amino acid sequence
(SEQ ID NO: 28)
ESIDSIYV,
a CDR2 amino acid sequence
(SEQ ID NO: 29)
LTPGGGS,
a CDR3 amino acid sequence
SEQ ID NO: 30)
NAFHRLRGTHY,
or
e)
a CDR1 amino acid sequence
(SEQ ID NO: 31)
GFTFQEAY,
a CDR2 amino acid sequence
(SEQ ID NO: 32)
IDSSVGST,
a CDR3 amino acid sequence
(SEQ ID NO: 33)
GTYDTEETTSSWEDEYQYAV.

In the above, group a) provides consensus sequences defined by antibodies herein termed sdAb021, sdAb022, sdAb023, sdAb024, sdAb025, sdAb026, and sdAb027.

“CDRs” or “complementarity-determining regions” are the portion of the variable chains in immunoglobulins that collectively constitute the paratope, and thereby impart binding specificity and affinity to the antibody. As used here, the term refers to CDRs mapped in sdAbs according to the standards or conventions set by IMGT™ (international ImMunoGeneTics information system).

The antibodies described herein have been raised to the human soluble EGFRvIII (e.g., see UniProt P00533: residues 1-29/Gly/297-645) or membrane-tethered human EGFRvII (e.g., see UniProt P00533: residues 1-29/Gly/297-668) expressed via DNA immunization of llama.

In another aspect, there is provided an isolated single domain antibody (sdAb), which binds specifically to human EGFR, the sdAb comprising:
A)
i) a CDR1 amino acid sequence as set forth in SEQ ID NO: 1, a CDR2 amino acid sequence as set forth in SEQ ID NO: 2, and a CDR3 amino acid sequence as set forth in SEQ ID NO: 3 (from NRC-sdAb021),
ii) a CDR1 amino acid sequence as set forth in SEQ ID NO: 4, a CDR2 amino acid sequence as set forth in SEQ ID NO: 5, and a CDR3 amino acid sequence as set forth in SEQ ID NO: 6 (from NRC-sdAb022),
iii) a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9 (from NRC-sdAb023),
iv) a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12 (from NRC-sdAb024),
v) a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15 (from NRC-sdAb025),
vi) a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18 (from NRC-sdAb026),
vii) a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (from NRC-sdAb027),
viii) a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (from NRC-sdAb028),
ix) a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 from (NRC-sdAb032),
x) a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30 (from NRC-sdAb033), or
xi) a CDR1 amino acid sequence as set forth in SEQ ID NO: 31, a CDR2 amino acid sequence as set forth in SEQ ID NO: 32, and a CDR3 amino acid sequence as set fort in SEQ ID NO: 33 (from NRC-VH54).

In one embodiment, the antibody comprises a CDR1 amino acid sequence as set forth in SEQ ID NO: 1, a CDR2 amino acid sequence as set forth in SEQ ID NO: 2, and a CDR3 amino acid sequence as set forth in SEQ ID NO: 3 (from NRC-sdAb02l).

In one embodiment, the antibody comprises a CDR1 amino acid sequence as set forth in SEQ ID NO: 4, a CDR2 amino acid sequence as set forth in SEQ ID NO: 5, and a CDR3 amino acid sequence as set forth in SEQ ID NO: 6 (from NRC-sdAb02l).

In one embodiment, the antibody comprises a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9 (from NRC-sdAb023).

In one embodiment, the antibody comprises a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12 (from NRC-sdAb024).

In one embodiment, the antibody comprises a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15 (from NRC-sdAb025).

In one embodiment, the antibody comprises a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18 (from NRC-sdAb026).

In one embodiment, the antibody comprises a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (from NRC-sdAb027)

In one embodiment, the antibody comprises a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (from NRC-sdAb028).

In one embodiment, the antibody comprises a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 (from NRC-sdAb032).

In one embodiment, the antibody comprises a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30 (from NRC-sdAb033).

In one embodiment, the antibody comprises a CDR1 amino acid sequence as set forth in SEQ ID NO: 31, a CDR2 amino acid sequence as set forth in SEQ ID NO: 32, and a CDR3 amino acid sequence as set forth in SEQ ID NO: 33 (from NRC-VH54).

In another aspect, there is provided an isolated single domain antibody (sdAb), which binds specifically to human EGFR, the sdAb comprising:
A)
i) a CDR1 amino acid sequence as set forth in SEQ ID NO: 1, a CDR2 amino acid sequence as set forth in SEQ ID NO: 2, and a CDR3 amino acid sequence as set forth in SEQ ID NO: 3 (from NRC-sdAb021),
ii) a CDR1 amino acid sequence as set forth in SEQ ID NO: 4, a CDR2 amino acid sequence as set forth in SEQ ID NO: 5, and a CDR3 amino acid sequence as set forth in SEQ ID NO: 6 (from NRC-sdAb022),
iii) a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9 (from NRC-sdAb023),
iv) a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12 (from NRC-sdAb024),
v) a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15 (from NRC-sdAb025),
vi) a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18 (from NRC-sdAb026),
vii) a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (from NRC-sdAb027),
viii) a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (from NRC-sdAb028),
ix) a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 (from NRC-sdAb032),
x) a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30 (from NRC-sdAb033), or
xi) a CDR1 amino acid sequence as set forth in SEQ ID NO: 31, a CDR2 amino acid sequence as set forth in SEQ ID NO: 32, and a CDR3 amino acid sequence as set fort in SEQ ID NO: 33 (from NRC-VH54); or
B)
CDR1, CDR2, and CDR3 amino acid sequences that are at least 80% identical to the CDR1, CDR2, and CDR3 sequences defined in any one of part A) i) to xi).

In one embodiment, in B) the CDR1 CDR2, and CDR3 amino acid sequences are at least 90% identical to the CDR1, CDR2, and CDR3 sequences defined in any one of part A) i) to xxviii). In one embodiment, in B) the CDR1 CDR2, and CDR3 amino acid sequences are at least 95% identical to the CDR1, CDR2, and CDR3 sequences defined in any one of part A) i) to xxviii). In one embodiment, in B) the CDR1 CDR2, and CDR3 amino acid sequences have at most three substitutions compared to the CDR1, CDR2, and CDR3 sequences defined in any one of part A) i) to xxviii). In one embodiment, in B) the CDR1 CDR2, and CDR3 amino acid sequences have at most two substitutions compared to the CDR1, CDR2, and CDR3 sequences defined in any one of part A) i) to xxviii). In one embodiment, in B) the CDR1 CDR2, and CDR3 amino acid sequences have at most one substitution compared to the CDR1, CDR2, and CDR3 sequences defined in any one of part A) i) to xxviii). In some embodiment, sequence differences vs. the sequences set forth in A) are conservative sequence substitutions.

The term “conservative amino acid substitutions” which is known in the art is defined herein as follows, with conservative substitutable candidate amino acids showing in parentheses: Ala (Gly, Ser); Arg (Gly, Gln); Asn (Gln; His); Asp (Glu); Cys (Ser); Gln (Asn, Lys); Glu (Asp); Gly (Ala, Pro); His (Asn; Gln); Ile (Leu; Val); Leu (lie; Val); Lys (Arg; Gln); Met (Leu, lie); Phe (Met, Leu, Tyr); Ser (Thr; Gly); Thr (Ser; Val); Trp (Tyr); Tyr (Trp; Phe); Val (lie; Leu).

Sequence variants according to certain embodiments are intended to encompass molecules in which binding affinity and/or specificity is substantially unaltered vs. the parent molecule from which it is derived. Such parameters can be readily tested, e.g., using techniques described herein and techniques known in the art. Such embodiments may encompass sequence substitutions, insertions, or deletions.

In another aspect, there is provided an isolated VHH single domain antibody (sdAb), which binds specifically to human EGFR, the sdAb comprising:
A)
a CDR3 amino acid sequence as set forth in SEQ ID NO: 3,
a CDR3 amino acid sequence as set forth in SEQ ID NO: 6,
a CDR3 amino acid sequence as set forth in SEQ ID NO: 9,
a CDR3 amino acid sequence as set forth in SEQ ID NO: 12,
a CDR3 amino acid sequence as set forth in SEQ ID NO: 15,
a CDR3 amino acid sequence as set forth in SEQ ID NO: 18,
a CDR3 amino acid sequence as set forth in SEQ ID NO: 21,
a CDR3 amino acid sequence as set forth in SEQ ID NO: 24,
a CDR3 amino acid sequence as set forth in SEQ ID NO: 27,
a CDR3 amino acid sequence as set forth in SEQ ID NO: 30, or
a CDR3 amino acid sequence as set forth in SEQ ID NO: 33.

Recognizing that CDR3 is often the major determinant of binding for V_{H}H sdAbs, it would be understood that other CDRs could be mutagenized or otherwise diversified and a resulting library (or candidate molecule) screened for antibodies that bind to human EGFR and/or cross-compete for binding to EGFR with the parent molecule. These embodiments are intended to cover, inter alia, molecules identified in this manner.

In one embodiment, the isolated sdAb comprises:
a CDR1 amino acid sequence as set forth in SEQ ID NO: 1, and a CDR3 amino acid sequence as set forth in SEQ ID NO: 3,
a CDR1 amino acid sequence as set forth in SEQ ID NO: 4, and a CDR3 amino acid sequence as set forth in SEQ ID NO: 6,
a CDR1 amino acid sequence as set forth in SEQ ID NO: 7, and a CDR3 amino acid sequence as set forth in SEQ ID NO: 9,
a CDR1 amino acid sequence as set forth in SEQ ID NO: 10, and a CDR3 amino acid sequence as set forth in SEQ ID NO: 12,
a CDR1 amino acid sequence as set forth in SEQ ID NO: 13, and a CDR3 amino acid sequence as set forth in SEQ ID NO: 15,
a CDR1 amino acid sequence as set forth in SEQ ID NO: 16, and a CDR3 amino acid sequence as set forth in SEQ ID NO: 18,
a CDR1 amino acid sequence as set forth in SEQ ID NO: 19, and a CDR3 amino acid sequence as set forth in SEQ ID NO: 21,
a CDR1 amino acid sequence as set forth in SEQ ID NO: 22, and a CDR3 amino acid sequence as set forth in SEQ ID NO: 24,
a CDR1 amino acid sequence as set forth in SEQ ID NO: 25, and a CDR3 amino acid sequence as set forth in SEQ ID NO: 27,
a CDR1 amino acid sequence as set forth in SEQ ID NO: 28, and a CDR3 amino acid sequence as set forth in SEQ ID NO: 30, or
a CDR1 amino acid sequence as set forth in SEQ ID NO: 31, and a CDR3 amino acid sequence as set forth in SEQ ID NO: 33.

In some embodiments, the sdAb cross-reacts with mouse EGFR.

In some embodiments, the sdAb cross-reacts with rhesus EGFR.

These embodiments are intended to encompass, inter alia, embodiments in which molecules recovered following mutagenization/diversification and screening for variant molecules that bind to EGFR and/or cross-compete for binding to EGFR with the parent molecule from which they are derived. As above, a library could be screened or individual candidate molecules could be tested.

In one embodiment, sdAb comprises A) the amino acid sequence of any one of SEQ ID NO: 34 to 48 across the full length thereof, or B) an amino acid sequence that is at least 80% identical to any one of SEQ ID NO: 34 to 48 across the full length thereof. In one embodiment, the amino acid sequence of B) is at least 85% identical across the full length therefore to one of the amino acid sequences of A). In one embodiment, the amino acid sequence of B) is at least 90% identical across the full length therefore to one of the amino acid sequences of A). In one embodiment, the amino acid sequence of B) is at least 95% identical across the full length therefore to one of the amino acid sequences of A). In one embodiment, the amino acid sequence of B) is at least 98% identical across the full length therefore to one of the amino acid sequences of A). In one embodiment, the amino acid sequence of B) is at least 98% identical across the full length therefore to one of the amino acid sequences of A). In some of these embodiments, sequences differences vs. sequences of A) are outside the CDR sequences.

In one embodiment, the sdAb comprises A) the amino acid sequence of any one of SEQ ID NO: 34 to 48.

In one embodiment, the sdAb comprises a CDR1, CDR2, and CDR3 of the sdAb sequence set forth in SEQ ID NO: 34.

In one embodiment, the sdAb comprises a CDR1, CDR2, and CDR3 of the sdAb sequence set forth in SEQ ID NO: 35.

In one embodiment, the sdAb comprises a CDR1, CDR2, and CDR3 of the sdAb sequence set forth in SEQ ID NO: 36.

In one embodiment, the sdAb comprises a CDR1, CDR2, and CDR3 of the sdAb sequence set forth in SEQ ID NO: 37.

In one embodiment, the sdAb comprises a CDR1, CDR2, and CDR3 of the sdAb sequence set forth in SEQ ID NO: 38.

In one embodiment, the sdAb comprises a CDR1, CDR2, and CDR3 of the sdAb sequence set forth in SEQ ID NO: 39.

In one embodiment, the sdAb comprises a CDR1, CDR2, and CDR3 of the sdAb sequence set forth in SEQ ID NO: 40.

In one embodiment, the sdAb comprises a CDR1, CDR2, and CDR3 of the sdAb sequence set forth in SEQ ID NO: 41.

In one embodiment, the sdAb comprises a CDR1, CDR2, and CDR3 of the sdAb sequence set forth in SEQ ID NO: 42.

In one embodiment, the sdAb comprises a CDR1, CDR2, and CDR3 of the sdAb sequence set forth in SEQ ID NO: 43.

In one embodiment, the sdAb comprises a CDR1, CDR2, and CDR3 of the sdAb sequence set forth in SEQ ID NO: 44.

In one embodiment, the sdAb comprises a CDR1, CDR2, and CDR3 of the sdAb sequence set forth in SEQ ID NO: 45.

In one embodiment, the sdAb comprises a CDR1, CDR2, and CDR3 of the sdAb sequence set forth in SEQ ID NO: 46.

In one embodiment, the sdAb comprises a CDR1, CDR2, and CDR3 of the sdAb sequence set forth in SEQ ID NO: 47.

In one embodiment, the sdAb comprises a CDR1, CDR2, and CDR3 of the sdAb sequence set forth in SEQ ID NO: 48.

In one embodiment of the above, CDR1, CDR2, and CDR3 are defined with respect to the IMGT numbering system. It is to be appreciated that CDR sequences could be defined by other conventions, such as the Kabat, Chothia, or EU numbering systems.

In one embodiment, the sdAb comprises SEQ ID NO: 34.

In one embodiment, the sdAb comprises SEQ ID NO: 35.

In one embodiment, the sdAb comprises SEQ ID NO: 36.

In one embodiment, the sdAb comprises SEQ ID NO: 37.

In one embodiment, the sdAb comprises SEQ ID NO: 38.

In one embodiment, the sdAb comprises SEQ ID NO: 39.

In one embodiment, the sdAb comprises SEQ ID NO: 40.

In one embodiment, the sdAb comprises SEQ ID NO: 41.

In one embodiment, the sdAb comprises SEQ ID NO: 42.

In one embodiment, the sdAb comprises SEQ ID NO: 43.

In one embodiment, the sdAb comprises SEQ ID NO: 44.

In one embodiment, the sdAb comprises SEQ ID NO: 45.

In one embodiment, the sdAb comprises SEQ ID NO: 46.

In one embodiment, the sdAb comprises SEQ ID NO: 47.

In one embodiment, the sdAb comprises SEQ ID NO: 48.

In one embodiment, the sdAb is a Camelidae V_{H}H sdAb.

In one embodiment, the sdAb is a llama V_{H}H sdAb

In one embodiment, the sdAb is humanized Camelidae V_{H}H.

By the term “humanized” as used herein is meant mutated so that immunogenicity upon administration in human patients is minor or nonexistent. Humanizing a polypeptide, according to the present invention, comprises a step of replacing one or more of the Camelidae amino acids by their human counterpart as found in the human consensus sequence, without that polypeptide losing its typical character, i.e. the humanization does not significantly affect the antigen binding capacity of the resulting polypeptide. A humanized antibody can be produced using a variety of techniques known in the art, including but not limited to, CDR-grafting, veneering or resurfacing, chain shuffling, etc.

In one embodiment, the humanized antibody comprises SEQ ID NO: 42 (NRC-sdAb028-H1).

In one embodiment, the humanized antibody comprises SEQ ID NO: 44 (NRC-sdAb032-H1).

In one embodiment, the humanized antibody comprises SEQ ID NO: 46 (NRC-sdAb033-H1).

In one embodiment, the humanized antibody comprises SEQ ID NO: 47 (NRC-sdAb033-H2).

In one embodiment, the sdAb has an affinity for human EGFR of 40 nM or less. In one embodiment, the sdAb has an affinity for human EGFR of 20 nM or less. In one embodiment, the sdAb has an affinity for human EGFR of 10 nM or less. In one embodiment, the sdAb has an affinity for human EGFR of 5 nM or less. Binding affinity can be determined, e.g., according to assays described herein.

In one aspect, there is provided a VHH single domain antibody (sdAb) that competes for specific binding to human EGFR with the isolated sdAb described above. An sdAb of the invention may be identified by a method that comprises a binding assay which assesses whether or not a test antibody is able to cross-compete with a known antibody of the invention for a binding site on the target molecule. For example, the antibodies described hereinabove may be used as reference antibodies. Methods for carrying out competitive binding assays are well known in the art. For example they may involve contacting together a known antibody of the invention and a target molecule under conditions under which the antibody can bind to the target molecule. The antibody/target complex may then be contacted with a test antibody and the extent to which the test antibody is able to displace the antibody of the invention from antibody/target complexes may be assessed. An alternative method may involve contacting a test antibody with a target molecule under conditions that allow for antibody binding, then adding an antibody of the invention that is capable of binding that target molecule and assessing the extent to which the antibody of the invention is able to displace the test antibody from antibody/target complexes. Such antibodies may be identified by generating new sdAbs to EGFR and screening the resulting library for cross-competition. Alternatively, one of the antibodies described herein may serve as a starting point for diversification, library generation, and screening. A further alternative could involve testing individual variants of an antibody described herein.

In one embodiment, the sdAb defined herein is a camelid sdAb.

In one embodiment, the sdAb defined herein is a llama sdAb.

In one embodiment, the sdAb defined herein is humanized form of Camelidae sdAb.

Table 1 and Table 7 lists the full length sequences for various sdAb disclosed herein. CDR1, CDR2, and CDR3 sequences are underlined in Table 7. CDR identification and numbering used herein is according to the IMGT™ convention.

Recombinant Polypeptides

In one aspect, there is provided a recombinant polypeptide comprising an sdAb as defined herein. In one embodiment, there is provided a recombinant polypeptide comprising one or more sdAb as defined herein. In one embodiment, there is provided a recombinant polypeptide comprising two or more sdAb as defined herein. In one embodiment, there is provided a recombinant polypeptide comprising two or more sdAb as defined herein.

V_{H}H:F_{c} Fusions

In one embodiment, there is provided the sdAb defined herein fused to a human Fc (termed a “V_{H}H:F_{c} fusion”). For example, the V_{H}H:F_{c} fusion may comprise at least a CH2 and a CH3 of the IgG, IgA, or IgD isotype. The V_{H}H:F_{c} fusion may comprise at least a CH2, a CH3, and a CH4 of the IgM or IgE isotype. Such embodiments may be useful in activating the immune system in higher order recombinant molecules. For example, according to some embodiments, two such F-containing V_{H}H:F_{c} fusions may assemble to form a recombinant monomeric antibody. In some embodiment, such a monomeric antibody is capable of activating the immune system. Such monomeric antibodies may be of IgG, IgA, IgD, IgE, or IgM isotype. In one embodiment, IgA F_{c}-containing V_{H}H:F_{c} fusions may also assemble into a recombinant dimeric (secretory) form. Multimeric forms are also envisaged in some embodiments. For example, five IgM monomers may assemble to form a recombinant pentameric antibody.

In some embodiments, the multivalent antibody described herein may be an assembly of the same VHH:Fc fusions.

In some embodiments, the multivalent antibody described herein may be an assembly of the different VHH:Fc fusions having the same binding target. For example, these may bind to different epitopes on the same target molecule. Examples may include assemblies of different VHH:Fc fusions, each comprising a different anti-EGFR sdAb as defined herein.

In some embodiments, the multivalent antibody described herein may be an assembly of an VHH:Fc fusion defined herein (comprising an anti-EGFR sdAb as defined herein) and another VHH:Fc fusion comprising a paratope directed to a different target.

Fusions to Cargo Molecules

In a further embodiment, the present disclosure provides an anti-EGFR sdAb as defined herein linked to a cargo molecule. The cargo molecule may comprise, for example, a therapeutic moiety, such as for example, a cytotoxic agent, a cytostatic agent, an anti-cancer agent or a radiotherapeutic. In particular embodiments of the disclosure, the antibody drug conjugates may comprise a cytotoxic agent. Another particular embodiment of the disclosure relates to antibody drug conjugates comprising a radiotherapeutic.

Targeted Nanoparticles

In another aspect, there is provided a nanoparticle linked to an EGFR sdAb as defined herein. The nanoparticle may comprise a therapeutic payload, such as a small molecule, polypeptide, or polynucleotide. The polynucleotide may comprise, for example, an mRNA, an siRNA, or an shRNA. The mRNA may encode a therapeutic polypeptide, such as a cytotoxic molecule. Such nanoparticles may be useful for targeted delivery of the therapeutic payload.

In one embodiment, there is provided a method of delivering a therapeutic payload to a cell, the method comprising contacting the cell with the nanoparticle linked to the EGFR sdAb. The cell may a cancer cell. The method may be a method of treating cancer in a subject.

In another embodiment, there is provided a use of the nanoparticle linked to the EGFR sdAb for delivery of a therapeutic payload to a cell. In another embodiment, there is the nanoparticle linked to the EGFR sdAb for use in delivery of a therapeutic payload to a cell. The cell may a cancer cell. The use may be for treatment of cancer in a subject.

Recombinant Nucleic Acid Molecules

In one aspect, there is provided a recombinant nucleic acid molecule encoding an sdAb, the recombinant polypeptide, or the V_{H}H:F_{c} fusion as defined herein. In one embodiment, the nucleic acid is DNA. In one embodiment, the nucleic acid is RNA. In one embodiment, the RNA is an mRNA. In one embodiment, the mRNA encodes for a therapeutic.

Compositions

In one aspect, there is provided a composition comprising an sdAb as defined herein, or a polypeptide comprising such an sdAb; together with an acceptable excipient, diluent or carrier. In one embodiment the composition is a pharmaceutical composition, and the excipient, diluent or carrier is a pharmaceutically acceptable excipient, diluent or carrier.

Uses & Methods

In one aspect, there is provided a use of the sdAb as defined herein or of an antibody comprising one or more V_{H}H:F_{c} fusion as defined herein for treatment of a cancer.

In one aspect, there is provided a use of the sdAb as defined herein or of an antibody comprising one or more V_{H}H:F_{c} fusion as defined herein for preparation of a medicament for treatment of a cancer.

In one aspect, there is provided the sdAb as defined herein or of an antibody comprising one or more V_{H}H:F_{c} fusion as defined herein for use in treatment of a cancer.

In one aspect, there is provided a method of treating a cancer in subject comprising administering to the subject the sdAb as defined herein or of an antibody comprising one or more V_{H}H:F_{c} fusion as defined herein.

In one embodiment, the cancer expresses EGFR. In one embodiment, the cancer aberrantly expresses EGFR normally absent in corresponding healthy tissue of origin and/or surrounding tissue. In one embodiment, the cancer expresses high levels of EGFR compared to corresponding healthy tissue of origin and/or surrounding tissue. In one embodiment, the cancer is selected from the group consisting of (but not limited to) lung cancer, pancreatic cancer, gastric cancer, brain cancer (such as glioblastoma), neuroendocrine cancer, head and neck cancer, bladder cancer, breast cancer, colorectal cancer, cervical cancer, and endometrial cancer.

Multivalent Antibodies & Related Embodiments

In one aspect, there is provided a multivalent antibody comprising an sdAb as defined above.

By “multivalent antibody” is use herein to mean a molecule comprising more than one variable region or paratope for binding to one or more antigen(s) within the same or different target molecule(s).

In some embodiments, the paratopes may bind to different epitopes on the same target molecule. In some embodiments, the paratopes may bind to different target molecules. In these embodiments, the multivalent antibody may be termed bispecific, trispecific, or multispecific, depending on the number of paratopes of different specificity that are present. As the multivalent antibody comprises one of the anti-EGFR sdAbs as herein defined, the multivalent antibody comprises EGFR binding affinity.

For example, as explained above, in some embodiments a multivalent antibody may be an assembly of a V_{H}H:F_{c} fusion defined herein (comprising an sdAb as defined herein) and another V_{H}H:F_{c} fusion comprising a different paratope conferring a different specificity.

In one embodiment, there is provided a bispecific antibody comprising an sdAb as defined above, and a second antigen-binding portion. In some embodiments, the second antigen binding portion may comprise a monoclonal antibody, an Fab, and F(ab′)₂, an Fab′, an scFv, or an sdAb, such as a V_{H}H or a V_{NAR}.

An “antigen-binding portion” is meant a polypeptide that comprises an antibody or antigen-binding fragment thereof having antigen-binding activity, including engineered antibodies fragments thereof.

In some embodiments, the second antigen-binding portion may bind to human serum albumin, e.g., for the purposes of stabilization/half-life extension.

In one embodiment, there is provided a trispecific antibody comprising an sdAb as defined above, and a second-binding portion, and a third antigen-binding portion. In some embodiments, the second antigen binding portion comprises a monoclonal antibody, an Fab, and F(ab′)₂, and Fab′, an sdFv, or an sdAb, such as a V_{H}H or a V_{NAR}. In some embodiments, the third antigen binding portion comprises, independently, a monoclonal antibody, an Fab, and F(ab′)₂, and Fab′, an sdFv, or an sdAb, such as a V_{H}H or a V_{NAR}.

The second and/or third antigen-binding portion may bind to human serum albumin, e.g., for the purposes of stabilization/half-life extension.

In some embodiments, the trispecific antibody may be multispecific and the antibody may comprise one or more additional antigen-binding portion(s). In such embodiments, the additional antigen-binding portion(s) may be, independently, an Fab, and F(ab′)₂, and Fab′, an sdFv, or an sdAb, such as a V_{H}H or a V_{NAR}.

In one embodiment, the multispecific antibody comprises a first antigen-binding portion comprising an sdAb as defined herein, and a second antigen-binding portion. In one embodiment, the second antigen-binding moiety binds specifically to a cell-surface marker of an immune cell.

A “cell surface marker” is a molecule expressed at the surface of the cell that is particular to (or enriched in) a cell type, and that is capable of being bound or recognized by an antigen-binding portion.

Bispecific T-Cell Engager

In one embodiment, the multivalent antibody is a bispecific T-cell engager comprising an sdAb as defined herein and second antigen-binding moiety that binds specifically to a cell-surface marker of a T-cell. In one embodiment, the T-cell marker comprises human CD3.

Human CD3 is a multi-subunit antigen, of which various subunits may participate in CD3 activation. One such subunit is CD3 epsilon (see, e.g., GenBank NP_000724.1). Other non-limiting examples include CD3 gamma (see, e.g., GenBank NP_000064.1) and delta (see, e.g., GenBank NP_000723.1 for delta isoform A, and, e.g., GenBank NP_001035741.1 for delta isoform B).

In some embodiments, T-cell marker comprises CD3 epsilon, CD3 gamma, or CD3 delta. In one specific embodiment, the T-cell marker comprises CD3 epsilon.

The term “bispecific T-cell engager”, as used herein, refers to a recombinant bispecific protein that has two linked variable regions from two different antibodies, one targeting a cell-surface molecule on T cells (for example, CD3ε), and the other targeting antigens on the surface of disease cells, typically malignant cells. For example a bispecific T-cell engager may comprises an sdAb as defined herein and an scFvs. A bispecific T-cell engager may comprise an sdAb as defined herein and a second VHH/sdAb. The two variable regions are typically linked together by a short flexible linker such as GlySer linker. By binding to tumor antigens and T cells simultaneously, bispecific T-cell engagers mediate T-cell responses and killing of tumor cells. The T-cell/target cell adherence facilitated by a bispecific T-cell engager is independent of MHC haplotype.

In one embodiment, the bispecific T-cell engager comprises in N-terminal to C-terminal direction:
the first antigen-binding portion,
an amino acid linker, and
the second antigen-binding portion.

In one embodiment, the signal peptide further comprises a signal peptide N-terminal to the fist antigen-binding portion.

A “signal peptide”, as referred to herein allows the nascent protein to be directed to the endoplasmic reticulum and subsequently to the cell surface, where it is expressed. The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases. The signal peptide may be at the amino terminus of the molecule.

In one embodiment, the signal peptide is a signal peptide from human CD28. In one embodiment, the signal peptide from human CD28 comprises SEQ ID NO: 53. In one embodiment, the signal peptide is at least 80% identical to SEQ ID NO: 53. In one embodiment, the signal peptide is at least 90% identical to SEQ ID NO: 53. In one embodiment, the signal peptide is at least 95% identical to SEQ ID NO: 53. In one embodiment, the signal peptide is at least 98% identical to SEQ ID NO: 53.

By “amino acid linker”, in this context, will be understood a sequence of sufficient length, flexibility, and composition to permit the bispecific T-cell engager to be properly functional an engage with both targets.

The amino acid linker may comprise a hinge. The hinge may be from human CD8, e.g. as set forth in SEQ ID NO: 55. In one embodiment, the CD8 hinge domain is at least 80% identical to SEQ ID NO: 55. In one embodiment, hinge domain is at least 90% identical to SEQ ID NO: 55. In one embodiment, the hinge domain is at least 95% identical to SEQ ID NO: 55. In one embodiment, the hinge domain is at least 98% identical to SEQ ID NO: 55.

The hinge may comprise a truncation of SEQ ID NO: 55. The hinge may comprise the C-terminal 22 amino acids of SEQ ID NO: 55 (SEQ ID NO: 57). The hinge may comprise the C-terminal 23 amino acids of SEQ ID NO: 55. The hinge may comprise the C-terminal 24 amino acids of SEQ ID NO: 55. The hinge may comprise the C-terminal 25 amino acids of SEQ ID NO: 55. The hinge may comprise the C-terminal 26 amino acids of SEQ ID NO: 55. The hinge may comprise the C-terminal 27 amino acids of SEQ ID NO: 55. The hinge may comprise the C-terminal 28 amino acids of SEQ ID NO: 55. The hinge may comprise the C-terminal 29 amino acids of SEQ ID NO: 55. The hinge may comprise the C-terminal 30 amino acids of SEQ ID NO: 55. The hinge may comprise the C-terminal 31 amino acids of SEQ ID NO: 55. The hinge may comprise the C-terminal 32 amino acids of SEQ ID NO: 55. The hinge may comprise the C-terminal 33 amino acids of SEQ ID NO: 55. The hinge may comprise the C-terminal 34 amino acids of SEQ ID NO: 55 (SEQ ID NO: 56). The hinge may comprise the C-terminal 35 amino acids of SEQ ID NO: 55. The hinge may comprise the C-terminal 36 amino acids of SEQ ID NO: 55. The hinge may comprise the C-terminal 37 amino acids of SEQ ID NO: 55. The hinge may comprise the C-terminal 38 amino acids of SEQ ID NO: 55. The hinge may comprise the C-terminal 39 amino acids of SEQ ID NO: 55. The hinge may comprise the C-terminal 40 amino acids of SEQ ID NO: 55. The hinge may comprise the C-terminal 41 amino acids of SEQ ID NO: 55. The hinge may comprise the C-terminal 42 amino acids of SEQ ID NO: 55. The hinge may comprise the C-terminal 43 amino acids of SEQ ID NO: 55. The hinge may comprise the C-terminal 44 amino acids of SEQ ID NO: 55. In one embodiment, the hinge comprises a sequence that is at least 80% identical to one of the aforementioned truncations. In one embodiment, the hinge comprises a sequence that is at least 90% identical to one of the aforementioned truncations. In one embodiment, the hinge comprises a sequence that is at least 95% identical to one of the aforementioned truncations. In one embodiment, the hinge comprises a sequence that is at least 98% identical to one of the aforementioned truncations.

In one embodiment, the amino acid linker comprises (in N- to C-terminal direction) SEQ ID NO: 54-SEQ ID NO: 55-SEQ ID NO: 62; or sequences at least 80% identical to SEQ ID NO: 54-SEQ ID NO: 55-SEQ ID NO: 62. In one embodiment, the amino acid linker comprises a sequence that is at least 80% identical to SEQ ID NO: 54. In one embodiment, the amino acid linker comprises a sequence that is at least 90% identical to SEQ ID NO: 54. In one embodiment, the amino acid linker comprises a sequence that is at least 95% identical to SEQ ID NO: 54. In one embodiment, the amino acid linker comprises a sequence that is at least 98% identical to SEQ ID NO: 54. In one embodiment, amino acid linker comprises a sequence that is at least 80% identical to SEQ ID NO: 55. In one embodiment, amino acid linker comprises a sequence that is at least 90% identical to SEQ ID NO: 55. In one embodiment, the amino acid linker comprises a sequence that is at least 95% identical to SEQ ID NO: 55. In one embodiment, the amino acid linker comprises a sequence that is at least 98% identical to SEQ ID NO: 55. In one embodiment, the amino acid linker comprises a sequence that is at least 80% identical to SEQ ID NO: 62. In one embodiment, the amino acid linker comprises a sequence that is at least 90% identical to SEQ ID NO: 62. In one embodiment, the amino acid linker comprises a sequence that is at least 95% identical to SEQ ID NO: 62. In one embodiment, the amino acid linker comprises a sequence that is at least 98% identical to SEQ ID NO: 62.

In one embodiment, the multivalent antibody is encoded by SEQ ID NO: 52.

In one embodiment, the sdAb comprises a CDR1 amino acid sequence as set forth in SEQ ID NO: 1, a CDR2 amino acid sequence as set forth in SEQ ID NO: 2, and a CDR3 amino acid sequence as set forth in SEQ ID NO: 3 (from NRC-sdAb021).

In one embodiment, the sdAb comprises a CDR1 amino acid sequence as set forth in SEQ ID NO: 4, a CDR2 amino acid sequence as set forth in SEQ ID NO: 5, and a CDR3 amino acid sequence as set forth in SEQ ID NO: 6 (from NRC-sdAb022).

In one embodiment, the sdAb comprises a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9 (from NRC-sdAb023).

In one embodiment, the sdAb comprises a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12 (from NRC-sdAb024).

In one embodiment, the sdAb comprises a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15 (from NRC-sdAb025).

In one embodiment, the sdAb comprises a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18 (from NRC-sdAb026).

In one embodiment, the sdAb comprises a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (NRC-sdAb027)

In one embodiment, the sdAb comprises a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (from NRC-sdAb028).

In one embodiment, the sdAb comprises a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 (from NRC-sdAb032).

In one embodiment, the sdAb comprises a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30 (from NRC-sdAb033).

In one embodiment, the sdAb comprises a CDR1 amino acid sequence as set forth in SEQ ID NO: 31, a CDR2 amino acid sequence as set forth in SEQ ID NO: 32, and a CDR3 amino acid sequence as set forth in SEQ ID NO: 33 (from NRC-VH54).

In one embodiment, the sdAb comprises a CDR1, CDR2, and CDR3 of the sdAb sequence set forth in SEQ ID NO: 34.

In one embodiment, the sdAb comprises a CDR1, CDR2, and CDR3 of the sdAb sequence set forth in SEQ ID NO: 35.

In one embodiment, the sdAb comprises a CDR1, CDR2, and CDR3 of the sdAb sequence set forth in SEQ ID NO: 36.

In one embodiment, the sdAb comprises a CDR1, CDR2, and CDR3 of the sdAb sequence set forth in SEQ ID NO: 37.

In one embodiment, the sdAb comprises a CDR1, CDR2, and CDR3 of the sdAb sequence set forth in SEQ ID NO: 38.

In one embodiment, the sdAb comprises a CDR1, CDR2, and CDR3 of the sdAb sequence set forth in SEQ ID NO: 39.

In one embodiment, the sdAb comprises a CDR1, CDR2, and CDR3 of the sdAb sequence set forth in SEQ ID NO: 40.

In one embodiment, the sdAb comprises a CDR1, CDR2, and CDR3 of the sdAb sequence set forth in SEQ ID NO: 41.

In one embodiment, the sdAb comprises a CDR1, CDR2, and CDR3 of the sdAb sequence set forth in SEQ ID NO: 42.

In one embodiment, the sdAb comprises a CDR1, CDR2, and CDR3 of the sdAb sequence set forth in SEQ ID NO: 43.

In one embodiment, the sdAb comprises a CDR1, CDR2, and CDR3 of the sdAb sequence set forth in SEQ ID NO: 44.

In one embodiment, the sdAb comprises a CDR1, CDR2, and CDR3 of the sdAb sequence set forth in SEQ ID NO: 45.

In one embodiment, the sdAb comprises a CDR1, CDR2, and CDR3 of the sdAb sequence set forth in SEQ ID NO: 46.

In one embodiment, the sdAb comprises a CDR1, CDR2, and CDR3 of the sdAb sequence set forth in SEQ ID NO: 47.

In one embodiment, the sdAb comprises a CDR1, CDR2, and CDR3 of the sdAb sequence set forth in SEQ ID NO: 48.

In one embodiment, the sdAb comprises SEQ ID NO: 34.

In one embodiment, the sdAb comprises SEQ ID NO: 35.

In one embodiment, the sdAb comprises SEQ ID NO: 36.

In one embodiment, the sdAb comprises SEQ ID NO: 37.

In one embodiment, the sdAb comprises SEQ ID NO: 38.

In one embodiment, the sdAb comprises SEQ ID NO: 39.

In one embodiment, the sdAb comprises SEQ ID NO: 40.

In one embodiment, the sdAb comprises SEQ ID NO: 41.

In one embodiment, the sdAb comprises SEQ ID NO: 42.

In one embodiment, the sdAb comprises SEQ ID NO: 43.

In one embodiment, the sdAb comprises SEQ ID NO: 44.

In one embodiment, the sdAb comprises SEQ ID NO: 45.

In one embodiment, the sdAb comprises SEQ ID NO: 46.

In one embodiment, the sdAb comprises SEQ ID NO: 47.

In one embodiment, the sdAb comprises SEQ ID NO: 48.

In some embodiments, the BiKE is a sequence variant of the above BiKE having 80%, 90%, 95%, 98%, or 99% identity to one of the above-described BiKEs. In some embodiments, the variant retains substantially the same binding specificity as the parent molecule from which it is derived. In some embodiments the variant retains substantially the same binding affinity as the parent molecule from which it is derived.

BiKEs & TriKEs

In one embodiment, the multivalent antibody is a bispecific killer cell engager.

The term “BiKE” refers to a recombinant bispecific protein that has two linked variable regions from two different antibodies, one targeting a cell-surface molecule on natural killer (NK) cells (for example, CD16), and the other targeting antigens on the surface of disease cells, typically malignant cells. For example the BiKE may comprises two scFvs, two VHHs, or a combination thereof. The two are typically linked together by a short flexible linker. By binding to tumor antigens and NK cells simultaneously, BiKEs mediate NK-cell responses and killing of tumor cells.

In one embodiment, the cell-surface marker of the immune cell comprises a natural killer (NK) cell marker. In one embodiment, the NK cell marker comprises human CD16.

In one embodiment, the multivalent antibody is a trispecific killer cell engager (BiKE).

The term “TriKE” indicates at a BiKE that has been further modified to include another functionality. This term has been used to encompass various approaches. One approach involves inserting an intervening immunomodulatory molecule (a modified human IL-15 crosslinker) to promote NK cell activation, expansion, and/or survival (Vallera et al. IL-15 Trispecific Killer Engagers (TriKEs) Make Natural Killer Cells Specific to CD33+ Targets While Also Inducing In Vivo Expansion, and Enhanced Function. Clinical Cancer Research. 2012; 22(14): 3440-50). Other TriKE approaches are trispecific molecules that include three antibody variable regions: one targeting an NK cell receptor and two that target tumour-associated antigens (Gleason et al. Bispecific and Trispecific Killer Cell Engagers Directly Activate Human NK Cells Through CD16 Signaling and Induce Cytotoxicity and Cytokine Production. Mol Cancer Ther. 2012; 11(12): 2674-84). Yet other TriKE approaches target two NK cell receptors (e.g., CD16 and NKp46) and one tumour-associated antigen (Gauthier et al. Multifunctional Natural Killer Cell Engagers Targeting NKp46 Trigger Protective Tumor Immunity. Cell. 2019; 177(7): 1701-13).

In one embodiment, the multivalent antibody further comprises a cytokine for stimulating activation, expansion, and/or survival of NK cells. In one embodiment, the cytokine for stimulating expansion of NK cells is interleukin-15 (IL15), a variant thereof, or a functional fragment thereof.

In one embodiment, the multivalent antibody further comprises at least a third antigen-binding portion that binds to a second NK cell marker. In one embodiment, the second NK cell marker is human NKp46.

In one embodiment, the multivalent antibody further comprises at least a third antigen-binding portion that binds to a tumour-associated antigen. In some embodiment, the tumour-associated antigen is distinct from human EGFR.

In one embodiment, the third antigen-binding portion comprises a V_{H}H, a V_{NAR}, or an scVF.

In one embodiment, the second antigen-binding portion comprises a V_{H}H.

In one embodiment, the third antigen-binding portion binds to human serum albumin. In such embodiment, the affinity for human serum albumin may contribute to stabilization/increased half-life.

In one embodiment, the sdAb comprises a CDR1 amino acid sequence as set forth in SEQ ID NO: 1, a CDR2 amino acid sequence as set forth in SEQ ID NO: 2, and a CDR3 amino acid sequence as set forth in SEQ ID NO: 3 (from NRC-sdAb021).

In one embodiment, the sdAb comprises a CDR1 amino acid sequence as set forth in SEQ ID NO: 4, a CDR2 amino acid sequence as set forth in SEQ ID NO: 5, and a CDR3 amino acid sequence as set forth in SEQ ID NO: 6 (from NRC-sdAb022).

In one embodiment, the sdAb comprises a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9 (from NRC-sdAb023).

In one embodiment, the sdAb comprises a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12 (from NRC-sdAb024).

In one embodiment, the sdAb comprises a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15 (from NRC-sdAb025).

In one embodiment, the sdAb comprises a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18 (from NRC-sdAb026).

In one embodiment, the sdAb comprises a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (from NRC-sdAb027)

In one embodiment, the sdAb comprises a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (from NRC-sdAb028).

In one embodiment, the sdAb comprises a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 (from NRC-sdAb032).

In one embodiment, the sdAb comprises a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30 (NRC-sdAb033).

In one embodiment, the sdAb comprises a CDR1 amino acid sequence as set forth in SEQ ID NO: 31, a CDR2 amino acid sequence as set forth in SEQ ID NO: 32, and a CDR3 amino acid sequence as set forth in SEQ ID NO: 33 (from NRC-VH54).

In one embodiment, the sdAb comprises a CDR1, CDR2, and CDR3 of the sdAb sequence set forth in SEQ ID NO: 34.

In one embodiment, the sdAb comprises a CDR1, CDR2, and CDR3 of the sdAb sequence set forth in SEQ ID NO: 35.

In one embodiment, the sdAb comprises a CDR1, CDR2, and CDR3 of the sdAb sequence set forth in SEQ ID NO: 36.

In one embodiment, the sdAb comprises a CDR1, CDR2, and CDR3 of the sdAb sequence set forth in SEQ ID NO: 37.

In one embodiment, the sdAb comprises a CDR1, CDR2, and CDR3 of the sdAb sequence set forth in SEQ ID NO: 38.

In one embodiment, the sdAb comprises a CDR1, CDR2, and CDR3 of the sdAb sequence set forth in SEQ ID NO: 39.

In one embodiment, the sdAb comprises a CDR1, CDR2, and CDR3 of the sdAb sequence set forth in SEQ ID NO: 40.

In one embodiment, the sdAb comprises a CDR1, CDR2, and CDR3 of the sdAb sequence set forth in SEQ ID NO: 41.

In one embodiment, the sdAb comprises a CDR1, CDR2, and CDR3 of the sdAb sequence set forth in SEQ ID NO: 42.

In one embodiment, the sdAb comprises a CDR1, CDR2, and CDR3 of the sdAb sequence set forth in SEQ ID NO: 43.

In one embodiment, the sdAb comprises a CDR1, CDR2, and CDR3 of the sdAb sequence set forth in SEQ ID NO: 44.

In one embodiment, the sdAb comprises a CDR1, CDR2, and CDR3 of the sdAb sequence set forth in SEQ ID NO: 45.

In one embodiment, the sdAb comprises a CDR1, CDR2, and CDR3 of the sdAb sequence set forth in SEQ ID NO: 46.

In one embodiment, the sdAb comprises a CDR1, CDR2, and CDR3 of the sdAb sequence set forth in SEQ ID NO: 47.

In one embodiment, the sdAb comprises a CDR1, CDR2, and CDR3 of the sdAb sequence set forth in SEQ ID NO: 48.

In one embodiment, the sdAb comprises SEQ ID NO: 34.

In one embodiment, the sdAb comprises SEQ ID NO: 35.

In one embodiment, the sdAb comprises SEQ ID NO: 36.

In one embodiment, the sdAb comprises SEQ ID NO: 37.

In one embodiment, the sdAb comprises SEQ ID NO: 38.

In one embodiment, the sdAb comprises SEQ ID NO: 39.

In one embodiment, the sdAb comprises SEQ ID NO: 40.

In one embodiment, the sdAb comprises SEQ ID NO: 41.

In one embodiment, the sdAb comprises SEQ ID NO: 42.

In one embodiment, the sdAb comprises SEQ ID NO: 43.

In one embodiment, the sdAb comprises SEQ ID NO: 44.

In one embodiment, the sdAb comprises SEQ ID NO: 45.

In one embodiment, the sdAb comprises SEQ ID NO: 46.

In one embodiment, the sdAb comprises SEQ ID NO: 47.

In one embodiment, the sdAb comprises SEQ ID NO: 48.

In some embodiments, the BiKE or TriKE is a sequence variant of one of the above BiKEs and TriKEs having 80%, 90%, 95%, 98%, or 99% identity thereto. In some embodiments, the variant retains substantially the same binding specificity as the parent molecule from which it is derived. In some embodiments the variant retains substantially the same binding affinity as the parent molecule from which it is derived.

In one embodiment, there is provided a multivalent antibody comprising: a first antigen-binding portion, an amino acid linker comprising a polypeptide hinge from human CD8, and a second antigen-binding portion. In one embodiment, the polypeptide hinge from human CD8 comprises SEQ ID NO: 55. In one embodiment, the amino acid linker further comprises at least one G4S positioned N-terminal to the polypeptide hinge from human CD8, and at least one G4S positioned C-terminal to the polypeptide hinge from human CD8. In one embodiment, the amino acid linker is at least 47 amino acids in length, preferably is at least 52 amino acids in length, preferably at least 57 amino acids in length, more preferably at least 62 amino acids in length, even more preferably at least 67 amino acids in length. In one embodiment, the amino acid linker comprises, in N-terminal to C-terminal, direction SEQ ID NOs: 54, 55, and 62. In one embodiment, the first antigen-binding portion binds specifically to human EGFR. In one embodiment, the first antigen-binding portion is a V_{H}H, V_{NAR}, or an scVF. In one embodiment, the first antigen-binding portion is one the anti-EGFR sdAbs as described herein. In one embodiment, the second antigen-binding moiety binds specifically to a cell-surface marker of an immune cell. In one embodiment, he cell-surface marker of the immune cell comprises a T-cell marker. In one embodiment, the T-cell marker comprises human CD3. In one embodiment, the second antigen binding portion is a V_{H}H, V_{NAR}, or an scVF.

Table 7 lists example sequences for and modules of multivalent antibodies described herein, according to certain aspects and embodiments. For the example bispecific T-cell engager construct comprising antibody EGFR1 (sdAb021) (SEQ ID NO: 69):
positions 1-21 correspond to the signal peptide
positions 22-135 correspond to antibody sdAb021 (but could, alternatively, be any other anti-EGFR sdAb described herein)
positions 136-143 correspond to restriction site scar
positions 144-211 correspond to the (G4S)3-hCD8a-(G4S) linker domain (also contains restriction sites for modular hinge)
positions 211 and 212 are separated by the heavy chain for a CD3-specific murine antibody
positions 212-227 correspond to the (G4S)3 linker, and
positions 227 and 228 are separated by correspond to the light chain for the CD3-specific murine antibody.

For the purposes of experimentation and testing, the construct thereafter comprises positions 228-234, which correspond to a 6×His tag and two stop codons.

Recombinant Nucleic Acid Molecules

In aspect, there is provided a recombinant nucleic acid molecule encoding the multivalent antibody as defined herein. In one embodiment, nucleic acid is a vector. In one embodiment, the nucleic acid is DNA. In one embodiment, the nucleic acid is RNA. In one embodiment, the RNA is an mRNA. In one embodiment, the mRNA encodes for a therapeutic.

Compositions

In one aspect, there is provided a composition comprising a multivalent antibody as defined herein; together with an acceptable excipient, diluent or carrier. In one embodiment, the composition comprises a bispecific T-cell engager as herein defined. In one embodiment, the composition comprises a BiKE as herein defined. In one embodiment, the composition comprises a TriKE as herein defined. In one embodiment the composition is a pharmaceutical composition, and the excipient, diluent or carrier is a pharmaceutically acceptable excipient, diluent or carrier.

Uses & Methods

In one aspect, there is provided a use of the multivalent antibody as defined herein for treatment of a cancer.

In one aspect, there is provided a use of the multivalent antibody as defined herein for preparation of a medicament for treatment of a cancer.

In one aspect, there is provided the multivalent antibody as defined herein for use in treatment of a cancer.

In one aspect, there is provided a method of treating a cancer in subject comprising administering to the subject the multivalent antibody as defined herein.

In one embodiment, the cancer expresses EGFR. In one embodiment, the cancer aberrantly expresses EGFR normally absent in corresponding healthy tissue of origin and/or surrounding tissue. In one embodiment, the cancer expresses high levels of EGFR compared to corresponding healthy tissue of origin and/or surrounding tissue. In one embodiment, the cancer is selected from the group consisting of (but not limited to) lung cancer, pancreatic cancer, gastric cancer, brain cancer (such as glioblastoma), neuroendocrine cancer, head and neck cancer, bladder cancer, breast cancer, colorectal cancer, cervical cancer, and endometrial cancer.

Chimeric Antibody Receptors & Related Embodiments

In one aspect, there is provided a chimeric antibody receptor (CAR), which binds to human EGFR, comprising the VHH sdAb as defined herein.

“Chimeric antigen receptors” are receptor proteins engineered to give T cells the new ability to target a specific protein. The receptors are chimeric because they combine both antigen-binding and T-cell activating functions into a single receptor (see Stoiber et al. Limitations in the Design of Chimeric Antigen Receptors for Cancer Therapy. Cells. 2012; 8(5): 472 and van der Stegen et al. The pharmacology of second-generation chimeric antigen receptors. Nat Rev Drug Discov. 2019; 14(7): 499-509).

In one embodiment, the CAR comprises, in N-terminal to C-terminal direction:
an EGFR binding domain comprising the sdAb as defined herein,
a polypeptide hinge,
a transmembrane domain, and
a cytoplasmic domain comprising a co-stimulatory domain and a signaling domain.

The term “polypeptide hinge” used herein generally means any oligo- or polypeptide that functions to link the extracellular ligand-binding domain to the transmembrane domain. In particular, hinge region are used to provide more flexibility and accessibility for the extracellular ligand-binding domain. A hinge region may comprise up to 300 amino acids, preferably 10 to 100 amino acids and most preferably 25 to 50 amino acids. Hinge region may be derived from all or part of naturally occurring molecules, such as from all or part of the extracellular region of CD8, CD4 or CD28, or from all or part of an antibody constant region. Alternatively the hinge region may be a synthetic sequence that corresponds to a naturally occurring hinge sequence, or may be an entirely synthetic hinge sequence.

In one embodiment, the polypeptide hinge comprises a CD8 hinge domain. In one embodiment, the CD8 hinge domain comprises SEQ ID NO: 55 or C-terminal fragment thereof comprising at least 22 C-terminal amino acids of SEQ ID NO: 55. In one embodiment, the CD8 hinge domain comprises SEQ ID NO: 55. In one embodiment, the CD8 hinge domain is at least 80% identical to SEQ ID NO: 55. In one embodiment, hinge domain is at least 90% identical to SEQ ID NO: 55. In one embodiment, the hinge domain is at least 95% identical to SEQ ID NO: 55. In one embodiment, the hinge domain is at least 98% identical to SEQ ID NO: 55.

The hinge may comprise a fragment of SEQ ID NO: 55. The hinge may comprise an C-terminal fragment of SEQ ID NO: 55. The hinge may comprise the C-terminal 22 amino acids of SEQ ID NO: 55 (SEQ ID NO: 57). The hinge may comprise the C-terminal 23 amino acids of SEQ ID NO: 55. The hinge may comprise the C-terminal 24 amino acids of SEQ ID NO: 55. The hinge may comprise the C-terminal 25 amino acids of SEQ ID NO: 55. The hinge may comprise the C-terminal 26 amino acids of SEQ ID NO: 55. The hinge may comprise the C-terminal 27 amino acids of SEQ ID NO: 55. The hinge may comprise the C-terminal 28 amino acids of SEQ ID NO: 55. The hinge may comprise the C-terminal 29 amino acids of SEQ ID NO: 55. The hinge may comprise the C-terminal 30 amino acids of SEQ ID NO: 55. The hinge may comprise the C-terminal 31 amino acids of SEQ ID NO: 55. The hinge may comprise the C-terminal 32 amino acids of SEQ ID NO: 55. The hinge may comprise the C-terminal 33 amino acids of SEQ ID NO: 55. The hinge may comprise the C-terminal 34 amino acids of SEQ ID NO: 55 (SEQ ID NO: 56). The hinge may comprise the C-terminal 35 amino acids of SEQ ID NO: 55. The hinge may comprise the C-terminal 36 amino acids of SEQ ID NO: 55. The hinge may comprise the C-terminal 37 amino acids of SEQ ID NO: 55. The hinge may comprise the C-terminal 38 amino acids of SEQ ID NO: 55. The hinge may comprise the C-terminal 39 amino acids of SEQ ID NO: 55. The hinge may comprise the C-terminal 40 amino acids of SEQ ID NO: 55. The hinge may comprise the C-terminal 41 amino acids of SEQ ID NO: 55. The hinge may comprise the C-terminal 42 amino acids of SEQ ID NO: 55. The hinge may comprise the C-terminal 43 amino acids of SEQ ID NO: 55. The hinge may comprise the C-terminal 44 amino acids of SEQ ID NO: 55. In one embodiment, the hinge comprises a sequence that is at least 80% identical to one of the aforementioned C-terminal fragments. In one embodiment, the hinge comprises a sequence that is at least 90% identical to one of the aforementioned C-terminal fragments. In one embodiment, the hinge comprises a sequence that is at least 95% identical to one of the aforementioned C-terminal fragments. In one embodiment, the hinge comprises a sequence that is at least 98% identical to one of the aforementioned C-terminal fragments.

In one embodiment, the polypeptide hinge comprises SEQ ID NO: 71 or a C-terminal fragment thereof comprising at least 22 amino acids.

In some embodiments, hinge length may be adjusted to achieve a desired property, as described herein. While, C-terminal fragments are discussed herein, it is also envisaged that N-terminal fragments or internal truncations (deletions) of SEQ ID NO: 71 could also be made and tested to determine which possess the desired property. These fragments or internal truncations may comprise at least 22 amino acids of SEQ ID NO: 71. They may comprise at least 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 amino acids of SEQ ID NO: 71.

In one embodiment, the hinge length may be selected to achieve maximum signaling, i.e. maximum activity toward target cells. Signaling can be assayed in vitro using, e.g., EGFR-high SKOV3 cells and, e.g., according to assays described herein. For example, a hinge comprising from 40 and 52 C-terminal amino acids of human of SEQ ID NO: 71 may achieve maximum signalling, e.g., for a CAR comprising a sdAb comprising a CDR1 of SEQ ID NO: 1, a CDR2 of SEQ ID NO: 2, and a CDR3 of SEQ ID NO:3; or wherein the CAR comprises SEQ ID NO: 34 (sdAb021). In some embodiments, a CAR construct comprising a hinge conveying these properties would be suited to targeting strategies that incorporate additional mechanisms to downregulate on-target off tumour responses. For example, these strategies may comprise Natural Killer-based cellular immunotherapy or Boolean-gated CAR receptors. The hinge may comprise 40, 41, 42, 43, 44, 45, 46, 47, 48, 49, 50, 51, or 52 C-terminal amino acids of SEQ ID NO: 71.

In one embodiment, the hinge length may be selected to maximize selectivity for target cells, i.e. discrimination for EGFR-high cells over EGFR-low cells. This may be tested, e.g., according to the assays described herein using, e.g., EGFR-high SKOV3 cells vs. EGFR-low MCF7 cells. For example, a hinge comprising from 34 to 39 C-terminal amino acids of human of SEQ ID NO: 71 may achieve maximum signalling, e.g., for a CAR comprising a sdAb comprising a CDR1 of SEQ ID NO: 1, a CDR2 of SEQ ID NO: 2, and a CDR3 of SEQ ID NO:3; or wherein the CAR comprises SEQ ID NO: 34 (sdAb021). In some embodiments, a CAR construct comprising a hinge conveying this property would be suited to “single CAR” applications, for example applications in which no other CAR or cellular regulation strategy is used. The hinge may comprise 34, 35, 36, 37, 38, or 39 C-terminal amino acids of SEQ ID NO: 71.

In one embodiment, the hinge length may be selected to achieve low activity towards target cells, i.e. some binding to EGFR-high target cells with negligible to no detectable signaling. This may be tested, e.g., according the assays described herein using, e.g., EGFR-high SKOV3 cells. For example, a hinge comprising from 22 to 33 C-terminal amino acids of human of SEQ ID NO: 55 may achieve maximum signalling, e.g., for a CAR comprising a sdAb comprising a CDR1 of SEQ ID NO: 1, a CDR2 of SEQ ID NO: 2, and a CDR3 of SEQ ID NO:3; or wherein the CAR comprises SEQ ID NO: 34 (sdAb021). In some embodiments, a CAR construct comprising a hinge conveying this properties would be suited for multi-antigen targeting CAR applications that incorporate additional targeting elements. These may include, but are not limited to, tandem CAR constructs containing multiple antigen binding domains, bi-cistronic CAR constructs wherein multiple CAR receptors are encoded in a single viral construct, co-transduction of CAR viruses targeting different antigens, or co-administration of separate CAR-T product which target different antigens. The hinge may comprises 22, 23, 24, 25, 26, 27, 28, 29, 30, 31, or 32 C-terminal amino acids of SEQ ID NO: 71.

The term “transmembrane domain” indicates a polypeptide having the ability to span a cell membrane and thereby link the extracellular portion of the CAR (which comprises the EGFR-binding portion) to the intracellular portion responsible for signaling. Commonly used transmembrane domains for CARs have been derived from CD4, CD8α, CD28 and CD3ζ.

In one embodiment, the transmembrane domain is a CD28 transmembrane domain. In one embodiment, the CD28 transmembrane domain comprises SEQ ID NO: 58. In one embodiment, the transmembrane domain is at least 80% identical to SEQ ID NO: 58. In one embodiment, the transmembrane domain is at least 90% identical to SEQ ID NO: 58. In one embodiment, the transmembrane domain is at least 95% identical to SEQ ID NO: 58. In one embodiment, the transmembrane domain is at least 98% identical to SEQ ID NO: 58.

The term “cytoplasmic domain” (also termed a “signal transduction domain”) refers to the intracellular portion of the CAR that is responsible for intracellular signaling following the binding of extracellular ligand binding domain to the target resulting in the activation of the immune cell and immune response. In other words, cytoplasmic domain is responsible for the activation of at least one of the normal effector functions of the immune cell in which the CAR is expressed. For example, the effector function of a T cell can be a cytolytic activity or helper activity including the secretion of cytokines. Thus, the term “cytoplasmic domain” refers to the portion of a protein which transduces the effector signal and directs the cell to perform a specialized function. It is common for such cytoplasmic domains to comprise a co-stimulatory domain in addition to a signaling domain.

The term “signaling domain” refers to the portion of a protein which transduces the effector signal and directs the cell to perform a specialized function. Examples of signal transducing domain for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. Signal transducing domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs or ITAMs. ITAMs are well defined signaling motifs found in the intracytoplasmic tail of a variety of receptors that serve as binding sites for syk/zap70 class tyrosine kinases. Non-limiting examples of signaling domains used in the invention can include those derived from TCRzeta, common FcR gamma (FCERIG), Fcgamma Rlla, FcRbeta (Fc Epsilon Rib), FcRepsilon, CD3 zeta, CD3gamma, CD3delta, CD3epsilon, CD5, CD22, CD79a, CD79b, CD66d, DAP10, or DAP12. In a preferred embodiment, the signaling transducing domain of the CAR can comprise the CD3zeta signaling domain.

In one embodiment, the signaling domain is a CD3-zeta signaling domain. In one embodiment, the CD3-zeta signaling domain comprises SEQ ID NO: 60. In one embodiment, the signaling domain is at least 80% identical to SEQ ID NO: 60. In one embodiment, the signaling domain is at least 90% identical to SEQ ID NO: 60. In one embodiment, the signaling domain is at least 95% identical to SEQ ID NO: 60. In one embodiment, the signaling domain is at least 98% identical to SEQ ID NO: 60.

The term “co-stimulatory domain” refers to the cognate binding partner on a T-cell that specifically binds with a co-stimulatory ligand, thereby mediating a co-stimulatory response by the cell, such as, but not limited to proliferation. Co-stimulatory molecules include, but are not limited to, an MHC class I molecule, BTLA and Toll ligand receptor. Examples of costimulatory molecules include CD27, CD28, 4-1 BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CDI Id, ITGAE, CD103, ITGAL, CDlla, LFA-1, ITGAM, CDllb, ITGAX, CDlIc, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D or a combination thereof.

In one embodiment, the co-stimulatory domain is a 4-1BB co-stimulatory domain. In one embodiment, the 4-1BB signal transduction domain comprises SEQ ID NO: 59. In one embodiment, the co-stimulatory domain is at least 80% identical to SEQ ID NO: 59. In one embodiment, the co-stimulatory domain is at least 90% identical to SEQ ID NO: 59. In one embodiment, the co-stimulatory domain is at least 95% identical to SEQ ID NO: 59. In one embodiment, the co-stimulatory domain is at least 98% identical to SEQ ID NO: 59.

In one embodiment, CAR further comprises a flexible amino acid linker between the sdAb and the polypeptide hinge. In one embodiment, the amino acid linker comprises SEQ ID NO: 62. In one embodiment, the amino acid linker is at least 80% identical to SEQ ID NO: 62. In one embodiment, the amino acid linker is at least 90% identical to SEQ ID NO: 62. In one embodiment, the amino acid linker is at least 95% identical to SEQ ID NO: 62. In one embodiment, the amino acid linker is at least 98% identical to SEQ ID NO: 62.

In one embodiment, the CAR further comprises a signal peptide.

In one embodiment, the signal peptide is a signal peptide from human CD28. In one embodiment, the signal peptide from human CD28 comprises SEQ ID NO: 53. In one embodiment, the signal peptide is at least 80% identical to SEQ ID NO: 53. In one embodiment, the signal peptide is at least 90% identical to SEQ ID NO: 53. In one embodiment, the signal peptide is at least 95% identical to SEQ ID NO: 53. In one embodiment, the signal peptide is at least 98% identical to SEQ ID NO: 53.

In one embodiment, the CAR is encoded by SEQ ID NO: 62.

In one embodiment, the sdAb comprises a CDR1 amino acid sequence as set forth in SEQ ID NO: 1, a CDR2 amino acid sequence as set forth in SEQ ID NO: 2, and a CDR3 amino acid sequence as set forth in SEQ ID NO: 3 (NRC-sdAb021).

In one embodiment, the sdAb comprises a CDR1 amino acid sequence as set forth in SEQ ID NO: 4, a CDR2 amino acid sequence as set forth in SEQ ID NO: 5, and a CDR3 amino acid sequence as set forth in SEQ ID NO: 6 (NRC-sdAb022).

In one embodiment, the sdAb comprises a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9 (NRC-sdAb023).

In one embodiment, the sdAb comprises a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12 (NRC-sdAb024).

In one embodiment, the sdAb comprises a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15 (NRC-sdAb025).

In one embodiment, the sdAb comprises a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18 (NRC-sdAb026).

In one embodiment, the sdAb comprises a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (NRC-sdAb027)

In one embodiment, the sdAb comprises a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (NRC-sdAb028).

In one embodiment, the sdAb comprises a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 (NRC-sdAb032).

In one embodiment, the sdAb comprises a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30 (NRC-sdAb033).

In one embodiment, the sdAb comprises a CDR1 amino acid sequence as set forth in SEQ ID NO: 31, a CDR2 amino acid sequence as set forth in SEQ ID NO: 32, and a CDR3 amino acid sequence as set forth in SEQ ID NO: 33 (NRC-VH54).

In one embodiment, the sdAb comprises a CDR1, CDR2, and CDR3 of the sdAb sequence set forth in SEQ ID NO: 34.

In one embodiment, the sdAb comprises a CDR1, CDR2, and CDR3 of the sdAb sequence set forth in SEQ ID NO: 35.

In one embodiment, the sdAb comprises a CDR1, CDR2, and CDR3 of the sdAb sequence set forth in SEQ ID NO: 36.

In one embodiment, the sdAb comprises a CDR1, CDR2, and CDR3 of the sdAb sequence set forth in SEQ ID NO: 37.

In one embodiment, the sdAb comprises a CDR1, CDR2, and CDR3 of the sdAb sequence set forth in SEQ ID NO: 38.

In one embodiment, the sdAb comprises a CDR1, CDR2, and CDR3 of the sdAb sequence set forth in SEQ ID NO: 39.

In one embodiment, the sdAb comprises a CDR1, CDR2, and CDR3 of the sdAb sequence set forth in SEQ ID NO: 40.

In one embodiment, the sdAb comprises a CDR1, CDR2, and CDR3 of the sdAb sequence set forth in SEQ ID NO: 41.

In one embodiment, the sdAb comprises a CDR1, CDR2, and CDR3 of the sdAb sequence set forth in SEQ ID NO: 42.

In one embodiment, the sdAb comprises a CDR1, CDR2, and CDR3 of the sdAb sequence set forth in SEQ ID NO: 43.

In one embodiment, the sdAb comprises a CDR1, CDR2, and CDR3 of the sdAb sequence set forth in SEQ ID NO: 44.

In one embodiment, the sdAb comprises a CDR1, CDR2, and CDR3 of the sdAb sequence set forth in SEQ ID NO: 45.

In one embodiment, the sdAb comprises a CDR1, CDR2, and CDR3 of the sdAb sequence set forth in SEQ ID NO: 46.

In one embodiment, the sdAb comprises a CDR1, CDR2, and CDR3 of the sdAb sequence set forth in SEQ ID NO: 47.

In one embodiment, the sdAb comprises a CDR1, CDR2, and CDR3 of the sdAb sequence set forth in SEQ ID NO: 48.

In one embodiment, the sdAb comprises SEQ ID NO: 34.

In one embodiment, the sdAb comprises SEQ ID NO: 35.

In one embodiment, the sdAb comprises SEQ ID NO: 36.

In one embodiment, the sdAb comprises SEQ ID NO: 37.

In one embodiment, the sdAb comprises SEQ ID NO: 38.

In one embodiment, the sdAb comprises SEQ ID NO: 39.

In one embodiment, the sdAb comprises SEQ ID NO: 40.

In one embodiment, the sdAb comprises SEQ ID NO: 41.

In one embodiment, the sdAb comprises SEQ ID NO: 42.

In one embodiment, the sdAb comprises SEQ ID NO: 43.

In one embodiment, the sdAb comprises SEQ ID NO: 44.

In one embodiment, the sdAb comprises SEQ ID NO: 45.

In one embodiment, the sdAb comprises SEQ ID NO: 46.

In one embodiment, the sdAb comprises SEQ ID NO: 47.

In one embodiment, the sdAb comprises SEQ ID NO: 48.

In some embodiments, the CAR is a sequence variant of one of the above CARs having 80%, 90%, 95%, 98%, or 99% identity thereto. In some embodiments, the variant retains substantially the same binding specificity as the parent molecule from which it is derived. In some embodiments the variant retains substantially the same binding affinity as the parent molecule from which it is derived.

Table 7 lists example sequences for and modules of multivalent antibodies and CARs described herein, according to certain aspects and embodiments.

For the example CAR construct comprising antibody EGFR1 (sdAb021) (SEQ ID NO: 66):
positions 1-21 correspond to the signal peptide,
positions 22-135 correspond to antibody sdAb021 (but could, alternatively, be any other anti-EGFR sdAb described herein),
positions 136-140 correspond to a restriction scar,
positions 141-185 correspond to a human CD8a hinge and restriction scars,
positions 186-217 correspond to the human CD28 transmembrane domain, and
positions 218-259 correspond to the human 41BB co-stimulatory domain, and
positions 260-373 correspond to human CD3-zeta signaling domain.

Nucleic Acids & Vectors

In one aspect, there is provided a recombinant nucleic acid molecule encoding the CAR as defined herein.

In one aspect, there is provided a vector comprising the recombinant nucleic acid molecule as defined herein. In one embodiment, the vector is a viral vector. In one embodiment, the viral vector is a lentivirus vector.

Viral Particles

In one aspect, there is provided a recombinant viral particle comprising the recombinant nucleic acid as defined herein. In one embodiment, the recombinant viral particle is a recombinant lentiviral particle.

Cells

In one aspect, there is provided a cell comprising the recombinant nucleic acid molecule as defined herein. In one embodiment, the recombinant nucleic acid molecule is DNA. In one embodiment, the recombinant nucleic acid molecule is RNA. In one embodiment, the RNA is mRNA. In one embodiment, the mRNA encodes a therapeutic.

In one embodiment, there is provided an engineered cell expressing at the cell surface membrane the CAR as defined herein. In one embodiment, the engineered cell is an immune cell. In one embodiment, the immune cell is a T-lymphocyte or is derived from T-lymphocytes.

Use & Methods

“CAR-T” cell therapy uses T cells engineered with CARs for cancer therapy. The premise of CAR-T immunotherapy is to modify T cells to recognize disease cells, typically cancer cells, in order to more effectively target and destroy them. Generally, T are genetically altered to express a CAR, and these cells are infused into a patient to attack their tumors. CAR-T cells can be either derived from T cells in a patient's own blood (autologous) or derived from the T cells of another healthy donor (allogeneic).

In one aspect, there is providing a use of the nucleic acid, vector, or viral particle as described herein for preparation of cells for CAR-T.

In one aspect, there is providing a method of preparing cells for CAR-T comprising contacting a T-cell with the viral particle as described herein. In one embodiment, the T-cell is from a donor. In one embodiment, the T-cell is from a patient.

In one aspect, there is providing a method of preparing cells for CAR-T comprising introducing into a T-cell the nucleic acid or vector as described herein. In one embodiment, the T-cell is from a donor. In one embodiment, the T-cell is from a patient.

In one aspect, there is provided a use of the CAR or of the engineered cell as described herein for treatment of a cancer.

In one embodiment, the method further comprises an initial step of obtaining cells from a patient or donor and introducing the recombinant nucleic acid molecule or vector encoding the CAR, as described herein.

In one embodiment, the method further comprises an initial step of obtaining cells from a patient or donor and contacting the cells with the viral particle, as described herein.

In one aspect, there is provided a use of the CAR or of the engineered cell as described herein for preparation of a medicament treatment of a cancer.

In one aspect, there is provided the CAR or the engineered cell as described herein for use in treatment of a cancer.

In one aspect there is provided a method of treating a cancer in a subject, comprising administering to the subject the engineered cell as defined herein.

In one embodiment, the cancer expresses EGFR. In one embodiment, the cancer aberrantly expresses EGFR normally absent in corresponding healthy tissue of origin and/or surrounding tissue. In one embodiment, the cancer expresses high levels of EGFR compared to corresponding healthy tissue of origin and/or surrounding tissue. In one embodiment, the cancer is selected from the group consisting of (but not limited to) lung cancer, pancreatic cancer, gastric cancer, brain cancer (such as glioblastoma), neuroendocrine cancer, head and neck cancer, bladder cancer, breast cancer, colorectal cancer, cervical cancer, and endometrial cancer.

### EXAMPLES

The following Examples outline embodiments of the invention and/or studies conducted pertaining to the invention. While the Examples are illustrative, the invention is in no way limited the following exemplified embodiments.

Abbreviations: Abs, antibodies; AF488, Alexa Fluor® 488; APC, allophycocyanin; BSA, bovine serum albumin; EGF, epidermal growth factor; EGFR, epidermal growth factor receptor; GPCR, G protein-coupled receptor; HBSS, Hank's Balanced Salt Solution; HRP, horseradish peroxidase; IMAC, immobilized metal affinity chromatography; LCIB, live cell imaging buffer; PBS, phosphate-buffered saline; PE, R-phycoerythrin; RU, resonance unit; sdAb, single-domain antibody; SPR, surface plasmon resonance; TMB, 3,3′,5,5′-tetramethylbenzidine; VHH, variable domain of camelid heavy chain-only antibody.

### Example 1

Generation and Characterization of Single-Domain Antibodies

In this Example, Camelid single-domain antibodies are raised to EGFR by DNA immunization.

Background

DNA immunization of large outbred animals is generally recognized as inconsistent and poorly effective in eliciting humoral immune responses. The mechanisms underlying this difficulty are thought to involve low rates of plasmid uptake and antigen expression, potentially relating to concentration effects in peripheral tissue. Studies have examined DNA immunization of camelid species, with generally poor results. Here, the hypothesis was tested that high-affinity and functional camelid sdAbs could be produced against a model antigen, EGFR, using DNA immunization alone. Herein is described the comprehensive in vitro characterization of a panel of sdAbs generated in this manner, which were dramatically superior to those previously isolated using protein immunization.

Materials and Methods

Antibodies and Reagents

Recombinant 6×His-tagged human EGFRvIII was produced by transient transfection of HEK293-6E cells as previously described [17] and purified by immobilized metal affinity chromatography (IMAC) followed by a final size exclusion chromatography polishing step to remove aggregates. Recombinant 6×His-tagged human EGFR ectodomain was from Genscript (Cat. No. Z03194; Piscataway, NJ), recombinant in vivo biotinylated 6×His-tagged human EGFRvIII ectodomain was from ACROBiosystems (Cat. No. EGR-H82E0; Newark, DE) and recombinant streptavidin was from Thermo Fisher Scientific (Waltham, MA). Human EGFR-Fc fusion protein was from Genscript (Cat. No. Z03381), and rhesus and mouse EGFR-Fc fusion proteins were from Sino Biological (Cat. Nos. 90317-K02H and 51091-M02H; Beijing, China). Horseradish peroxidase (HRP)-conjugated goat polyclonal anti-llama IgG was from Cedarlane Laboratories (Cat. No. A160-100P; Burlington, Canada), HRP-conjugated goat polyclonal anti-human IgG was from Sigma-Aldrich (St. Louis, MO) and 3,3′,5,5′-tetramethylbenzidine (TMB) substrate was from Mandel Scientific (Guelph, Canada). Bovine serum albumin (BSA) and Tween-20 were from Sigma-Aldrich, and all cell culture reagents were from Thermo Fisher. Mouse monoclonal anti-c-Myc IgG was from Santa Cruz Biotechnology (clone 9E10, Cat. No. sc-40; Dallas, TX), allophycocyanin (APC)-conjugated goat polyclonal anti-mouse IgG was from Thermo Fisher (Cat. No. A865), Alexa Fluor® 488 (AF488)-conjugated donkey anti-human IgG was from Jackson ImmunoResearch (Cat. No. 709546098; West Grove, PA) and R-phycoerythrin (PE)-conjugated streptavidin was from Thermo Fisher (Cat. No. S866). Erlotinib was from Sigma-Aldrich, recombinant human epidermal growth factor (EGF) and mouse monoclonal Ab against β-actin were from Genscript (Cat. Nos. Z00333 and A00702), rabbit polyclonal Ab against phospho-EGFR (Tyr1068) was from Cell Signaling Technology (Cat. No. 2234; Danvers, MA), mouse monoclonal Ab against human EGFR was a generous gift from Anne Marcil (National Research Council Canada, Montreal, Canada) and cetuximab was a generous gift from Yves Durocher (National Research Council Canada, Montreal, Canada). HRP-conjugated donkey polyclonal anti-mouse IgG was from Jackson ImmunoResearch (Cat. No. 715036150) and HRP-conjugated goat polyclonal anti-rabbit IgG was from Cedarlane Laboratory (Cat. No. CLCC43007). SuperSignal™ West Pico PLUS chemiluminescent substrate was from Thermo Fisher.

Llama Immunization

A male llama (Lama glama) was immunized by biolistic transfection using a Helios® gene gun system (Bio-Rad, Hercules, CA) followed by intradermal injection using a DERMOJET device (AKRA DERMOJET, Pau, France). Two pTT5 vectors [17] encoding either soluble human EGFRvII (UniProt P00533: residues 1-29/Gly/297-645) or membrane-tethered human EGFRvII (UniProt P00533: residues 1-29/Gly/297-668) were purified from overnight cultures of Escherichia coli DH5α cells using a QIAGEN® Plasmid Maxi Kit (Qiagen, Hilden, Germany). Briefly, 50 mg of gold particles were coated with 100 μL of 0.05 M spermidine, then vortexed and sonicated. An equimolar mixture of both pTT5 vectors (50 pg each; 100 pg total DNA in 100 μL ultrapure water) was added to the spermidine-coated gold particles, then 100 μL of 1 M CaCl2 was added dropwise to the mixture. After incubating for 10 min at room temperature, the gold particles were pelleted in a microfuge, washed three times with 100% ethanol and resuspended in 6 ml of 100% ethanol containing 0.05 mg/ml polyvinylpyrrolidone. The DNA-gold solution was dried onto the inner walls of two 30-inch lengths of gold-coat tubing under nitrogen flow, then the tubing was cut into 0.5-inch lengths.

The llama was immunized six times (weeks 0, 2, 4, 6, 9 and 12) by biolistic transfection; each immunization consisted of 12 bombardments administered at 600 PSI to shaved sites on the neck and hind limb (10 pg of total DNA per immunization). Thereafter, four additional immunizations (weeks 16, 20, 24 and 28) were administered by intradermal injection of 1 mg (1 mg/ml) of DNA using a DERMOJET device. Serum titration ELISA was conducted as described previously [18], and binding was detected using HRP-conjugated polyclonal goat anti-llama IgG. Experiments involving animals were conducted using protocols approved by the National Research Council Canada Animal Care Committee and in accordance with the guidelines set out in the OMAFRA Animals for Research Act, R. S. O. 1990, c. A.22.

Construction and Panning of Phage-Displayed V_{H}H Library

A phage-displayed VHH library was constructed from the peripheral blood lymphocytes of the immunized llama as described previously [18]. Briefly, peripheral blood mononuclear cells were purified by density gradient centrifugation from blood obtained 5 days following the third and the final DERMOJET immunizations. Total RNA was extracted from −5×10⁷ cells from each time point using a PureLink™ RNA Mini Kit (Thermo Fisher) and cDNA was reverse transcribed using qScript® cDNA supermix containing random hexamer and oligo(dT) primers (Quanta Biosciences, Gaithersburg, MD). VHH genes were amplified using semi-nested PCR and cloned into the phagemid vector pMED1; the final library had a size of 3×10⁷ independent transformants and an insert rate of −75%. Phage particles were rescued from the library using M13K07 helper phage and panned against microplate-adsorbed human EGFRvIII for three rounds with triethylamine elution as described previously [18]. A second independent library selection was carried out in the same manner, except that the target was streptavidin-captured biotinylated EGFRvIII.

Expression of V_{H}Hs and V_{H}H-Fc Fusions

VHH DNA sequences were cloned into the pSJF2H expression vector and monomeric VHHs tagged C-terminally with c-Myc and 6×His were purified from the periplasm of E. coli TG1 cells by IMAC as previously described [18]. In addition, in vivo biotinylated monomeric VHHs were produced by co-transformation of E. coli BL21 (DE3) cells with two vectors encoding (i) VHHs C-terminally tagged with a biotin acceptor peptide and 6×His and (ii) the biotin ligase BirA and purified by IMAC [19]. Bivalent VHH-human IgG1 Fc fusions were produced by transient transfection of HEK293-6E cells followed by protein A affinity chromatography as previously described [17]. Heterodimeric biparatopic VHH-Fc fusions were produced by co-transfection of HEK293-6E cells with two pTT5 vectors encoding (i) NRC-sdAb032-Fc tagged C-terminally with 6×His and (ii) a second untagged VHH-Fc. The heterodimeric Ab was purified by sequential protein A affinity chromatography and IMAC and eluted using a linear 0→0.5 M imidazole gradient over 20 column volumes to separate species bearing one or two 6×His tags. VHHs and VHH-Fcs were dialyzed against or buffer-exchanged into phosphate-buffered saline (PBS), pH 7.4.

ELISA and EGF-Competition ELISA

Wells of NUNCO MaxiSorp™ microtiter plates (Thermo Fisher) were coated overnight at 4° C. with 2 pg/ml streptavidin in 100 μL of PBS, pH 7.4. The wells were blocked with 200 μL of PBS containing 1% (w/v) BSA for 1 h at 37° C. and then biotinylated VHHs [10 pg/ml in 100 μL of PBS containing 1% BSA and 0.1% (v/v) Tween-20] were captured for 30 min at room temperature. The wells were washed 5× with PBS containing 0.1% Tween-20 and then incubated with human EGFR-Fc (500 ng/ml in 100 μL of PBS containing 1% BSA and 0.05% Tween-20) in the presence or absence of EGF (17 pg/ml) for 1 h at room temperature. The wells were washed 5× again and incubated with HRP-conjugated goat anti-human IgG (1 pg/ml in 100 μL of PBS containing 1% BSA and 0.05% Tween-20) for 1 h at room temperature. After a final wash (5× with PBS containing 0.1% Tween-20), the wells were developed with TMB substrate, stopped with 1 M _{H2SO4} and the absorbance at 450 nm was measured using a Multiskan™ FC photometer (Thermo Fisher).

Surface Plasmon Resonance

Prior to surface plasmon resonance (SPR) analyses, monomeric VHHs were purified by preparative size exclusion chromatography using a Superdex™ 75 10/300 GL column (GE Healthcare, Piscataway, NJ) connected to an AKTA FPLC protein purification system (GE Healthcare). In the first SPR experiment, multi-cycle kinetic analyses were performed on a Biacore™ 3000 instrument (GE Healthcare) at 25° C. in HBS-EP buffer [10 mM HEPES, pH 7.4, containing 150 mM NaCl, 3 mM EDTA and 0.005% (w/v) surfactant P20]. Approximately 1304-2158 and 741 resonance units (RUs), respectively, of recombinant human EGFR and EGFRvIII ectodomains were immobilized on a CM5 sensor chip (GE Healthcare) in 10 mM acetate buffer, pH 4.5, using an amine coupling kit (GE Healthcare). An ethanolamine-blocked flow cell served as the reference. Monomeric VHHs at concentrations ranging from 0.1 to 100 nM were injected over the EGFR and EGFRvIII surfaces in HBS-EP buffer at a flow rate of 20 μL/min. For NRC-sdAb032 only, the VHH (212 RUs) was immobilized on a CM5 sensor chip by amine coupling and 0.5-50 nM recombinant human EGFR ectodomain was injected over the VHH surface in HBS-EP buffer at a flow rate of 20 μL/min. Contact times were 180-300 s and dissociation times were 300-600 s. The EGFR, EGFRvIII and NRC-sdAb032 surfaces were regenerated using a 10 s pulse of 10 mM glycine, pH 1.5.

In the second SPR experiment, single-cycle kinetic analyses were performed on a Biacore™ T200 instrument (GE Healthcare) at 25° C. in HBS-EP buffer. Approximately 618, 1051 and 600 RUs of human, rhesus and mouse EGFR-Fc, respectively, were immobilized on three flow cells of a Series S Sensor Chip CM5 (GE Healthcare) in 10 mM acetate buffer, pH 4.5, using an amine coupling kit. An ethanolamine-blocked flow cell served as the reference. Monomeric VHHs at concentrations ranging from 0.6 to 50 nM were injected over the EGFR surfaces in HBS-EP buffer at a flow rate of 40 μL/min. The contact time was 180 s and the dissociation time was 600 s. The EGFR surfaces were regenerated using a 10 s pulse of 10 mM glycine, pH 1.5.

Epitope-binning experiments were performed essentially as described above on a Biacore™ 3000 instrument, except that 3383 RUs of human EGFR-Fc were immobilized on a CM5 sensor chip. A single VHH (or cetuximab) at a concentration equivalent to 25×KD was injected at 40 μL/min with a contact time of 150 s to saturate the EGFR surface. The second injection consisted of the same VHH (or cetuximab) in the presence of 25× the KD concentration of a second VHH. All data were analyzed by fitting to a 1: 1 interaction model using BIAevaluation 4.1 software (GE Healthcare).

Flow Cytometry and Mirrorball® Assays

MDA-MB-468 and MCF7 cells were cultured at 37° C. in a humidified 5% C02 atmosphere in T75 flasks containing RPMI-1640 medium supplemented with 10% (v/v) fetal bovine serum, 2 mM glutamine, 100 U/ml penicillin, 100 g/ml streptomycin and 250 ng/ml amphotericin B. For flow cytometry experiments, cells were grown to 70-80% confluency, dissociated using trypsin-EDTA solution, washed in PBS and then resuspended in PBS containing 1% BSA. Approximately 1×10⁵ cells were stained sequentially on ice for 30 min with (i) 20 pg/ml of each VHH, (ii) 5 pg/ml of mouse anti-c-Myc IgG and (iii) 5 pg/ml of APC-conjugated goat anti-mouse IgG. The cells were washed with PBS in between each staining step, and after a final wash, data were acquired on a BD FACSCanto™ instrument (BD Biosciences, San Jose, CA).

For Mirrorball® experiments, cells were dissociated from flasks using Accutase® solution, washed in Hank's Balanced Salt Solution (HBSS) and then ˜5000 cells in growth medium were plated in each rat tail collagen-coated well of a Nunc™ MicroWell 96-well optical bottom plate (Thermo Fisher). After incubating at 37° C./5% C02 for 24 h, the cells were washed with HBSS and Abs [biotinylated VHHs, VHH-Fcs or cetuximab, serially diluted in live cell imaging buffer (LCIB) containing 1% BSA] were added to wells for 2 h at 4° C. The cells were washed with LCIB and secondary detection reagents (40 pg/ml PE-conjugated streptavidin or 30 pg/ml AF488-conjugated donkey anti-human IgG in LCIB containing 1% BSA) were added as appropriate to each well for 1 h at 4° C. The cells were washed with LCIB and stained with 1 M DRAQ5™ for 10 min at 4° C. After a final wash with LCIB, data were acquired on a Mirrorball® microplate cytometer (TTP Labtech, Melbourn, U.K.) and analyzed using Cellista software (TTP Labtech).

EGFR Phosphorylation Assay

Approximately 2×10⁵ MDA-MB-468 cells were seeded in wells of 12-well tissue culture plates and then starved in serum-free RPMI-1640 medium overnight. The next day, the medium was replaced with RPMI-1640 containing 1% BSA and various concentrations of erlotinib or Abs (VHH-Fcs or cetuximab). After 30 min at 37° C., EGF was added to a final concentration of 25 nM and incubated for a further 15 min. The cells were cooled immediately on ice, washed twice with PBS and then scraped in 100 ml Laemmli buffer. Cell lysates (5 ml) were electrophoresed on 4-20% Mini-Protean® TGX™ precast gels (Bio-Rad Laboratories) and transferred to polyvinylidene fluoride membranes using the semi-dry method. Western blotting was performed as previously described [6]. Briefly, membranes were blocked overnight at 4° C. with 2% BSA in PBS, then sequentially incubated for 1 h at room temperature with (i) primary Abs (anti-phospho-EGFR, anti-EGFR or anti-β-actin, all diluted 1: 1500 in PBS containing 1% BSA and 0.1% Tween-20) and (ii) secondary Abs (HRP-conjugated donkey anti-mouse IgG or goat anti-rabbit IgG, diluted 1: 3000 in PBS containing 1% BSA and 0.1% Tween-20). The membranes were washed extensively with PBS containing 0.1% Tween-20 following incubations with primary and secondary Abs. The blots were developed using enhanced chemiluminescence and imaged using a Molecular Imager® Gel Doc™ XR+ System (Bio-Rad Laboratories). Band densitometry analysis was conducted using ImageJ version 1.52.

V_{H}H Humanization

VHHs were humanized by alignment with human IGHV3-30*01 and IGHJ1-1*01 amino acid sequences. For each VHH, three humanized variants were designed representing a spectrum of increasing homology to the human germline: (i) variant H1, in which all FR sequences were reverted to the human consensus excepting residues located within five positions of an FR-CDR boundary; (ii) variant H2, in which all FR sequences were reverted to the human consensus excepting residues located within two positions of an FR-CDR boundary and (iii) variant H3, in which all FR sequences were fully human. CDR residues as well as FR2 positions 42 and 52 (IMGT numbering) were left unaltered in all variants.

Results and Discussion

Llama DNA Immunization Using Gene Gun and DERMOJET

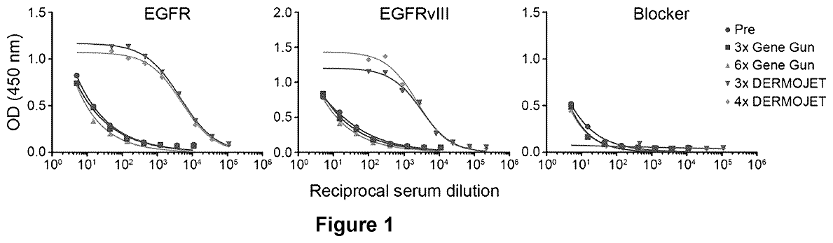

A llama was immunized with DNA encoding human EGFRvIII six times every 2-3 weeks by biolistic transfection using a gene gun. No polyclonal serum Ab response was evident in the animal following the six gene gun immunizations, but boosting three times by intradermal injection using a DERMOJET device elicited serum Abs against recombinant EGFR with a half-maximal titer of ˜1: 5000 (FIG. 1). Binding of polyclonal serum Abs to EGFR and EGFRvIII was roughly equivalent and was not improved by further boosting.

V_{H}Hs Elicited by DNA Immunization Targeted Five Unique EGFR Epitopes Including an Epitope Overlapping that of Cetuximab

A phage-displayed VHH library was constructed from the peripheral blood lymphocytes of the immunized llama and VHHs were isolated by panning against either plate-adsorbed EGFRvIII or streptavidin-captured biotinylated EGFRvIII. Ten unique VHHs falling into three sequence families were identified in the panning against plate-adsorbed EGFRvIII (NRC-sdAb021-NRC-sdAb030; Table 1).

TABLE 1
Sequence characteristics of anti-EGFR
V_{H}Hs raised by llama DNA immunization

| Family | V_{H}H | CDR-H3 Length (aa)^{a} | Alternative Name |
|---|---|---|---|
| — | EG2 | 18 | — |
| 1 | NRC-sdAb021 | 8 | EGFR1 |
| | NRC-sdAb022 | 8 | EGFR2 |
| | NRC-sdAb023 | 8 | EGFR3 |
| | NRC-sdAb024 | 8 | EGFR4 |
| | NRC-sdAb025 | 8 | EGFR5 |
| | NRC-sdAb026 | 8 | EGFR6 |
| | NRC-sdAb027 | 8 | EGFR7 |
| 2 | NRC-sdAb028 | 10 | EGFR10 |
| 3 | NRC-sdAb029 | 17 | — |
| | NRC-sdAb030 | 17 | — |
| 4 | NRC-sdAb032 | 12 | EVE46 |
| 5 | NRC-sdAb033 | 11 | EVE48 |

^{a}IMGT framework definitions

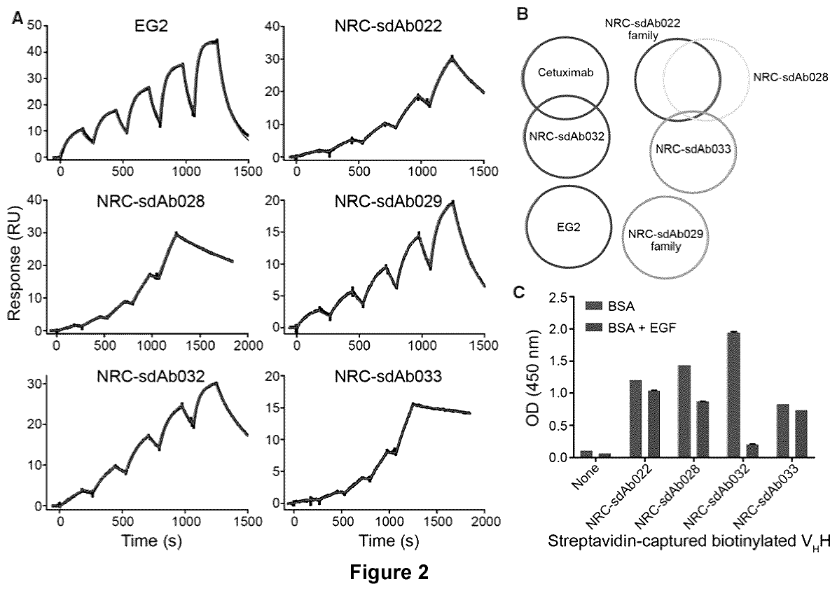

All of these VHHs as well as two others (NRC-sdAb032 and NRC-sdAb033) were identified by panning against streptavidin-captured biotinylated EGFRvIII. The VHHs had monovalent binding affinities for recombinant human EGFR, ranging from 1 to 40 nM as measured by SPR, and, despite immunization with DNA encoding EGFRvIII, all showed identical binding to EGFR and EGFRvIII (FIG. 2, Panel A and Table 2).

TABLE 2
Monovalent affinities and kinetics of the interactions between VHHs and recombinant
human EGFR and EGFRvIII extracellular domains (pH 7.4, 25° C.)

| | EGFR | | | EGFRvIII | | |
|---|---|---|---|---|---|---|
| V_{H}H | kₒₙ (M⁻¹s⁻¹) | k_{off} (s⁻¹) | K_{D} (nM) | kₒₙ (M⁻¹s⁻¹) | k_{off} (s⁻¹) | K_{D} (nM) |
| EG2 | 1.1 × 10⁶ | 2.8 × 10⁻² | 19.1 | 1.2 × 10⁶ | 1.7 × 10⁻² | 14.6 |
| NRC-sdAb021 | 3.1 × 10⁵ | 1.2 × 10⁻² | 38.5 | 2.4 × 10⁵ | 9.8 × 10⁻³ | 40.4 |
| NRC-sdAb022 | 4.0 × 10⁵ | 6.7 × 10⁻⁴ | 1.7 | 2.9 × 10⁵ | 7.7 × 10⁻⁴ | 2.6 |
| NRC-sdAb023 | 4.6 × 10⁵ | 5.7 × 10⁻³ | 12.6 | 3.4 × 10⁵ | 6.7 × 10⁻³ | 19.4 |
| NRC-sdAb024 | 2.5 × 10⁵ | 7.3 × 10⁻⁴ | 2.9 | 1.8 × 10⁵ | 9.0 × 10⁻⁴ | 4.9 |
| NRC-sdAb025 | 3.6 × 10⁵ | 5.9 × 10⁻⁴ | 1.7 | 2.6 × 10⁵ | 7.1 × 10⁻⁴ | 2.8 |
| NRC-sdAb026 | 3.6 × 10⁵ | 5.6 × 10⁻³ | 15.4 | 2.7 × 10⁵ | 6.8 × 10⁻³ | 25.0 |
| NRC-sdAb027 | 5.1 × 10⁵ | 7.9 × 10⁻⁴ | 1.6 | 3.8 × 10⁵ | 8.6 × 10⁻⁴ | 2.3 |
| NRC-sdAb028 | 8.4 × 10⁴ | 7.5 × 10⁻⁴ | 9.0 | 9.8 × 10⁴ | 8.4 × 10⁻⁴ | 8.6 |
| NRC-sdAb029 | 4.0 × 10⁵ | 4.0 × 10⁻³ | 9.9 | 4.4 × 10⁵ | 4.4 × 10⁻³ | 10.2 |
| NRC-sdAb030 | 2.7 × 10⁵ | 5.9 × 10⁻³ | 21.4 | 2.9 × 10⁵ | 6.6 × 10⁻³ | 22.7 |
| NRC-sdAb032 | 1.0 × 10⁵ | 1.3 × 10⁻³ | 12.9¹ | n.d.² | n.d.² | n.d.² |
| NRC-sdAb033 | 1.5 × 10⁵ | 1.7 × 10⁻⁴ | 1.1 | n.d.² | n.d.² | n.d.² |

¹Determined by amine-coupling NRC-sdAb032 and flowing recombinant EGFR ectodomain.
²No difference was observed in ELISA binding to human EGFR or EGFRvIII (data not shown); n.d., not determined.

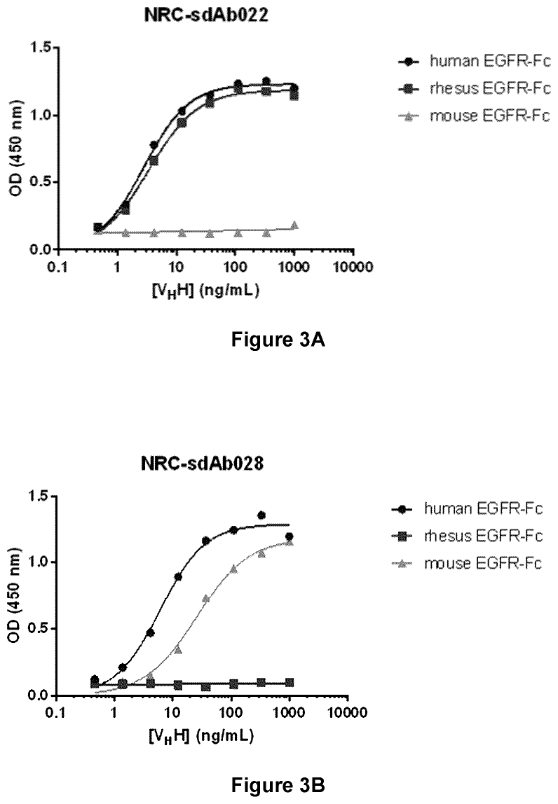

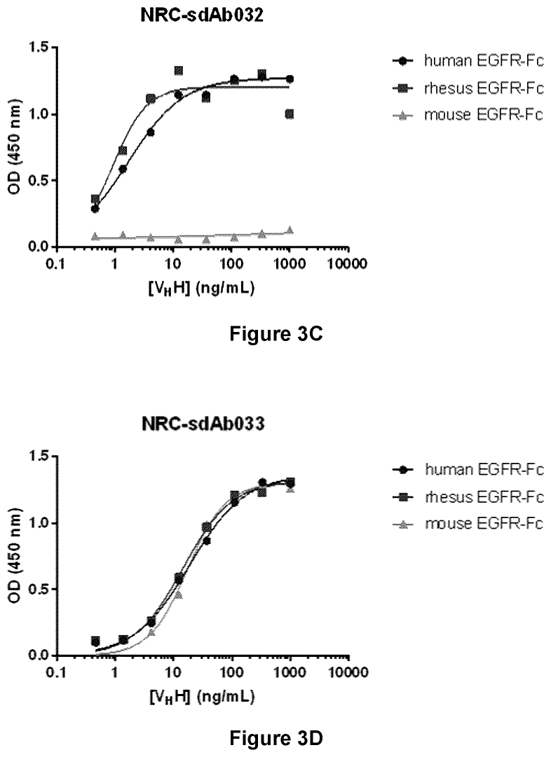

In contrast with a previous report, the EGFR-binding affinity of EG2 was measured (a VHH raised by immunization with the recombinant EGFRvIII ectodomain) as ˜15-20 nM, not 55 nM [4]. The VHHs showed a variety of cross-species reactivity patterns (Tables 3A and 3B, and FIG. 3).

TABLE 3A
Monovalent affinities and kinetics of the interactions between
VHHs and human, rhesus and mouse EGFR-Fc (pH 7.4, 25° C.)

| | Human EGFR-Fc | | | Rhesus EGFR-Fc | |
|---|---|---|---|---|---|
| | kₒₙ | | | kₒₙ | |
| V_{H}H | (M⁻¹ s⁻¹) | k_{off} (s⁻¹) | K_{D} (nM) | (M⁻¹ s⁻¹) | k_{off} (s⁻¹) |
| EG2 | 8.5 × 10⁵ | 8.5 × 10⁻³ | 11.1 ± 0.2 | — | — |
| NRC- | 2.5 × 10⁵ | 2.2 × 10⁻³ | 8.9 ± 0.1 | 8.5 × 10⁴ | 1.7 × 10⁻³ |
| sdAb022 | | | | | |
| NRC- | 9.2 × 10⁴ | 5.5 × 10⁻⁴ | 6.0 ± 0.1 | — | — |
| sdAb028 | | | | | |
| NRC- | 5.3 × 10⁵ | 4.5 × 10⁻³ | 8.5 ± 0.1 | 5.5 × 10⁵ | 6.4 × 10⁻³ |
| sdAb029 | | | | | |
| NRC- | 3.6 × 10⁵ | 2.5 × 10⁻³ | 6.9 ± 0.02 | 4.8 × 10⁵ | 3.9 × 10⁻³ |
| sdAb032 | | | | | |
| NRC- | 9.0 × 10⁴ | 1.6 × 10⁻⁴ | 1.8 ± 0.1 | 1.1 × 10⁵ | 1.1 × 10⁻⁴ |
| sdAb033 | | | | | |

(—) indicates no binding.
K_{D} values are expressed as the means ± standard deviations of three independent experiments.

TABLE 3B
(continuation of Table 3A) Monovalent affinities
and kinetics of the interactions between VHHs and
human, rhesus and mouse EGFR-Fc (pH 7.4, 25° C.)

| | Mouse EGFR-Fc | | | |
|---|---|---|---|---|
| V_{H}H | K_{D} (nM) | kₒₙ (M⁻¹ s⁻¹) | k_{off} (s⁻¹) | K_{D} (nM) |
| EG2 | — | — | — | — |
| NRC-sdAb022 | 19.6 ± 0.7 | — | — | — |
| NRC-sdAb028 | — | 6.5 × 10⁵ | 7.5 × 10⁻³ | 11.6 ± 0.3 |
| NRC-sdAb029 | 11.8 ± 0.3 | 5.1 × 10⁵ | 6.3 × 10⁻³ | 12.5 ± 1.2 |
| NRC-sdAb032 | 8.1 ± 0.1 | — | — | — |
| NRC-sdAb033 | 1.0 ± 0.1 | 9.2 × 10⁴ | 2.8 × 10⁻⁴ | 3.1 ± 0.2 |

(—) indicates no binding.
K_{D} values are expressed as the means ± standard deviations of three independent experiments.

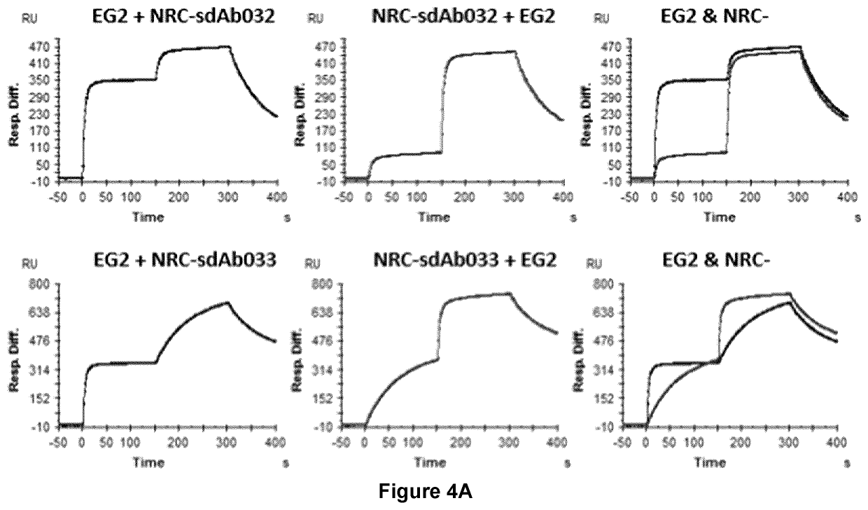

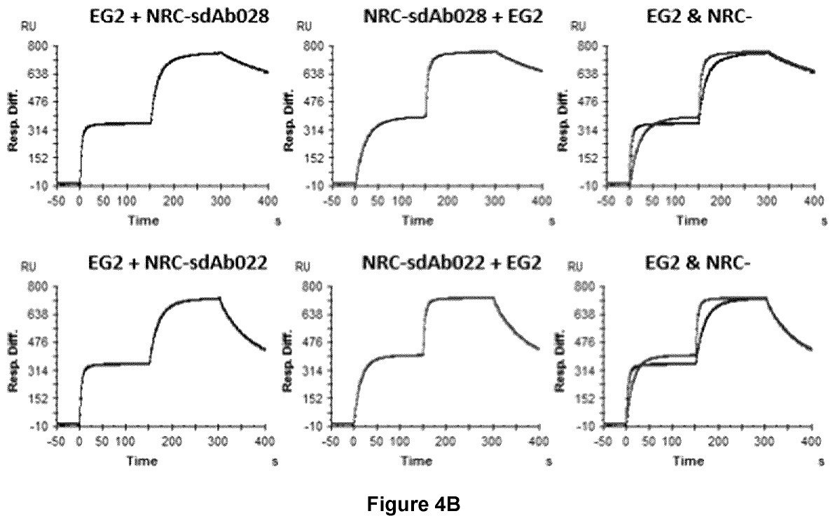

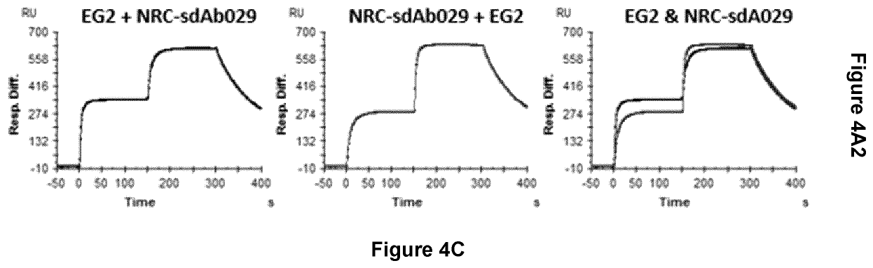

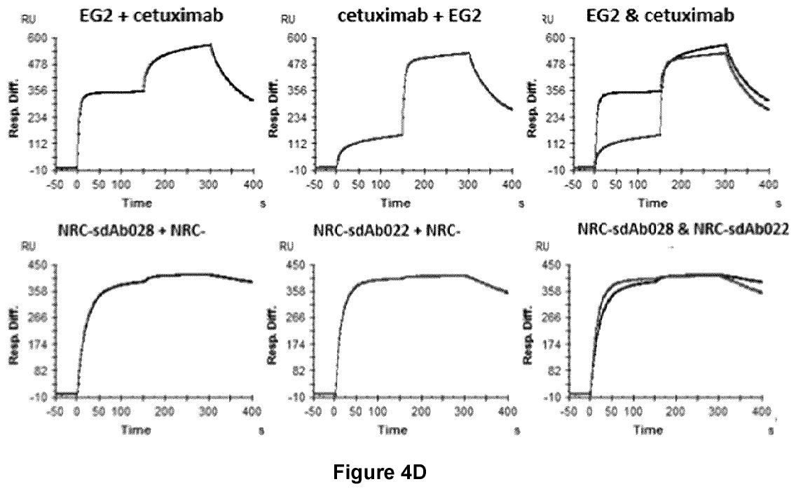

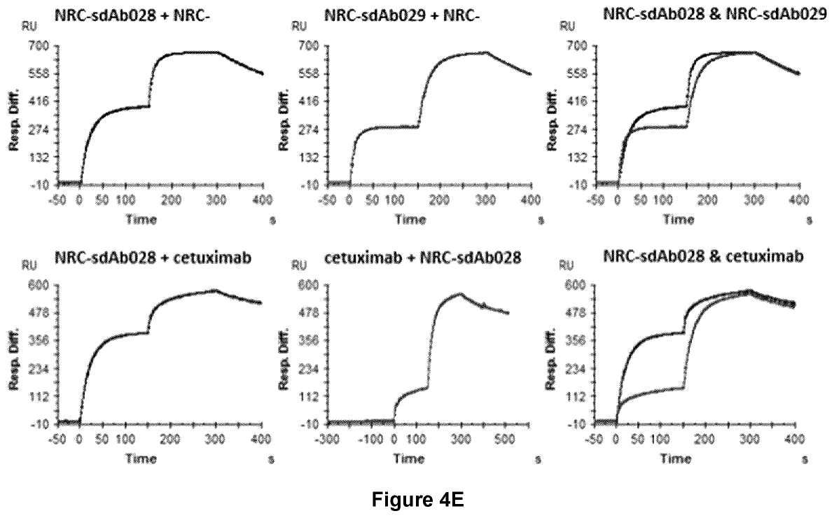

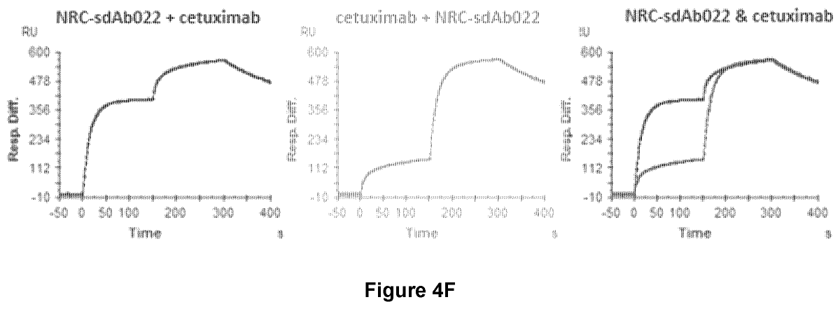

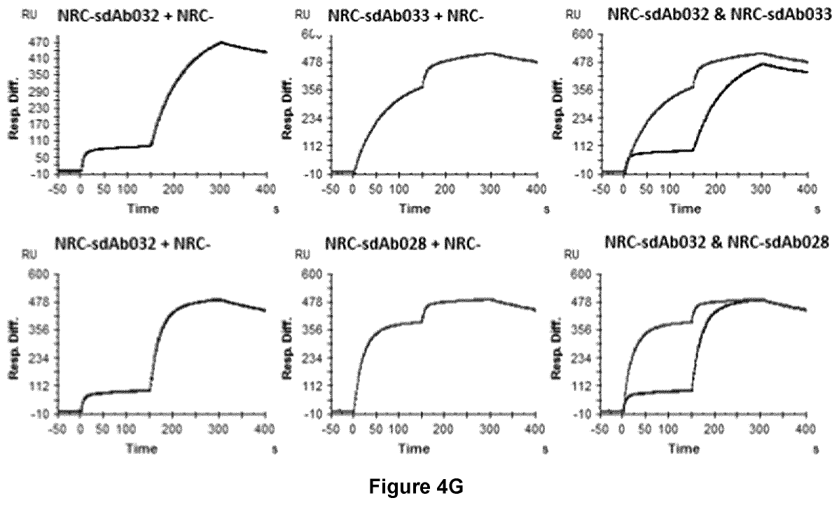

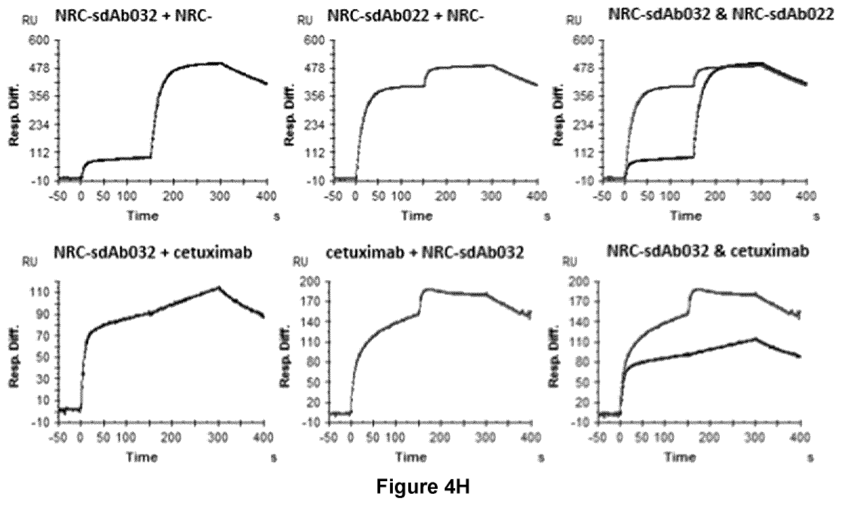

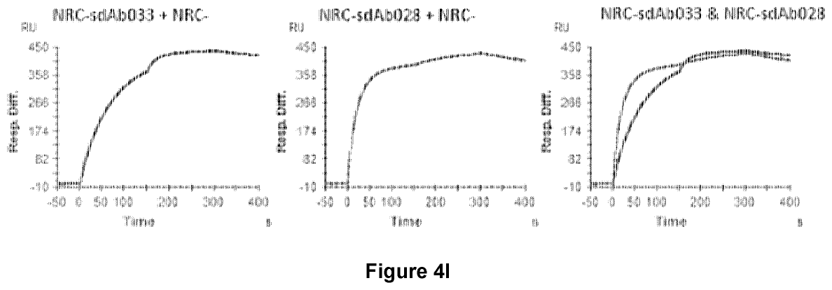

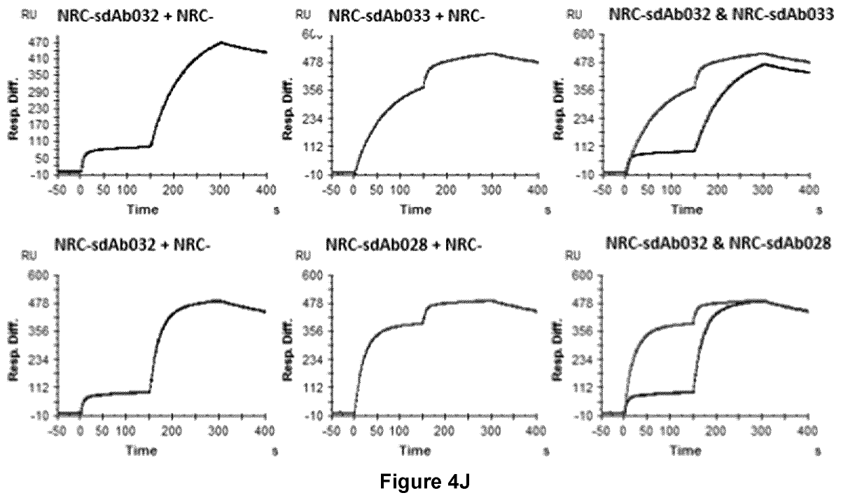

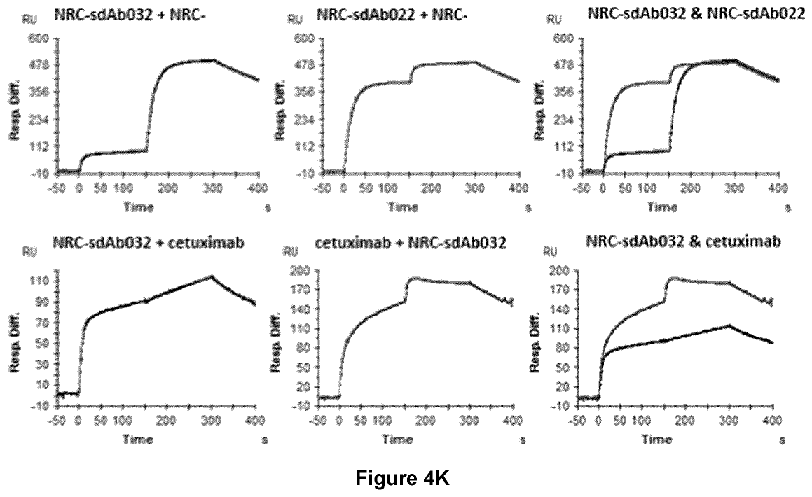

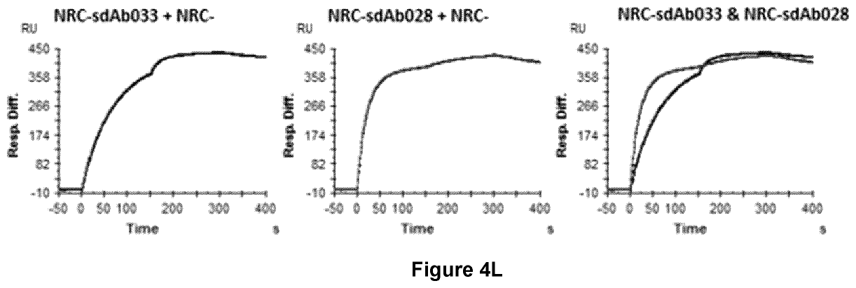

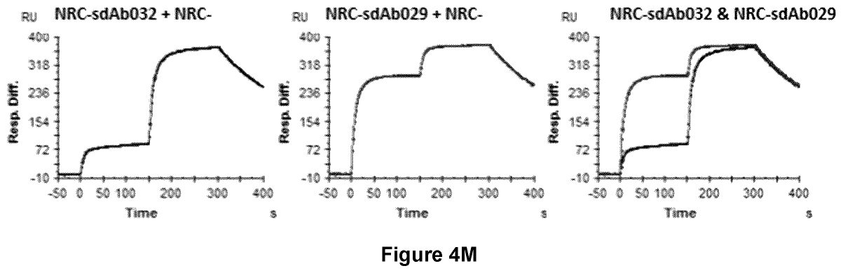

EG2 VHH bound only human EGFR, NRC-sdAb022-family VHHs and NRC-sdAb032 bound human and rhesus EGFR with similar affinities, and NRC-sdAb029-family VHHs and NRC-sdAb033 bound human, rhesus and mouse EGFR with similar affinities (FIGS. 3A, 3C, and 3D). Surprisingly, NRC-sdAb028 bound human and mouse EGFR with similar affinity but did not react with rhesus EGFR (FIG. 3B). SPR epitope-binning co-injection experiments showed that: (i) EG2 VHH recognized a distinct epitope present only on human EGFR; (ii) NRC-sdAb029-family VHHs recognized a distinct epitope conserved across human, rhesus and mouse EGFR; (iii) NRC-sdAb022-family VHHs and NRC-sdAb028 recognized highly overlapping but distinct epitopes (conserved across human and rhesus EGFR and human, rhesus and mouse EGFR, respectively), while NRC-sdAb033 recognized a partially overlapping but highly conserved epitope, and (iv) NRC-sdAb032 recognized an epitope partially overlapping that of cetuximab (FIG. 2, Panel B, and FIGS. 4A to 4M). NRC-sdAb032 shared further similarities with cetuximab, in that both Abs cross-reacted with rhesus but not mouse EGFR, neither Ab bound well in SPR to immobilized EGFR but did bind EGFR in solution (data not shown), and both Abs competed with EGF for EGFR binding (FIG. 2, Panel C).

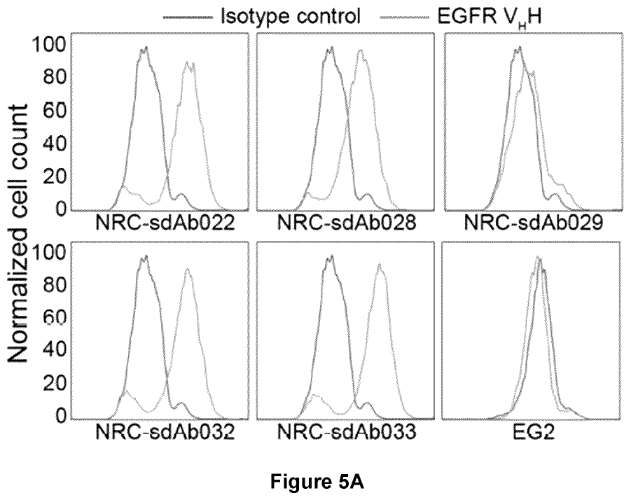

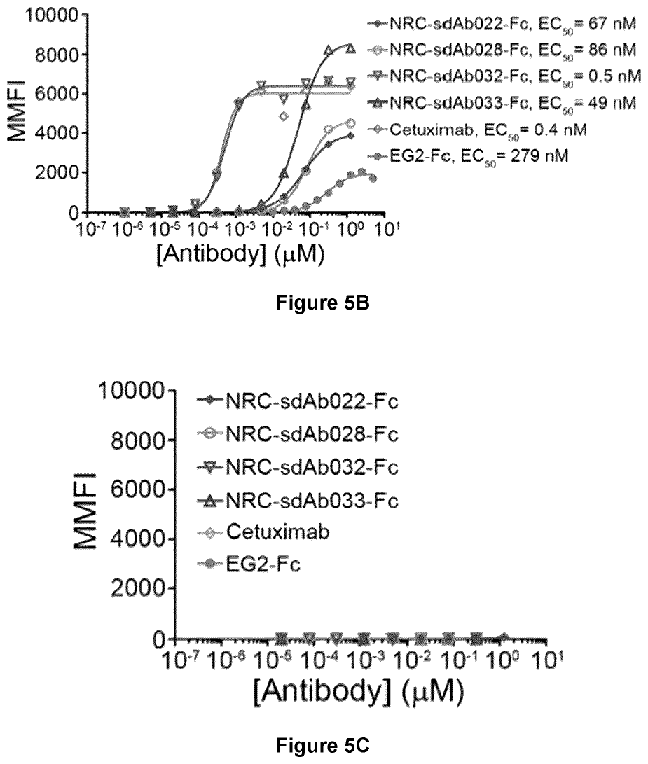

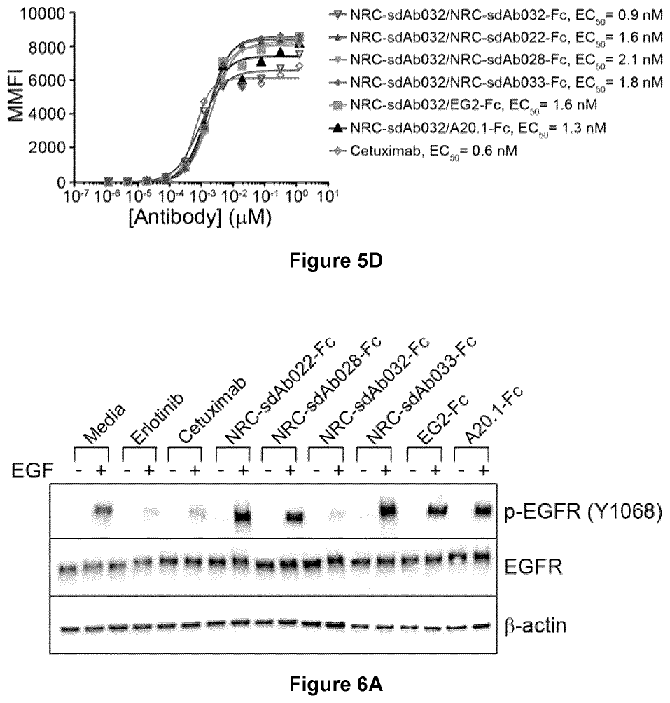

Despite their broadly similar monovalent affinities for recombinant EGFR (1-40 nM), the VHHs showed significant variability in their ability to recognize EGFR-positive tumor cell lines. Flow cytometry showed that four of five epitope bins targeted by the VHH monomers were accessible on native cell-surface EGFR; neither binding of NRC-sdAb029 nor, surprisingly, binding of EG2 to MDA-MB-468 cells was detectable at the single concentration (20 mg/ml, equivalent to ˜1.3 mM) used in this assay (FIG. 5, Panel A). Titration of the VHH-Fc fusions against adherent MDA-MB-468 cells used in Mirrorball® microplate cytometry assay revealed very weak binding of EG2-Fc (EC50: 279 nM), moderate binding of NRC-sdAb022-Fc, NRC-sdAb028-Fc and NRC-sdAb033-Fc (EC50: 67, 86 and 49 nM, respectively) and strong binding of NRC-sdAb032 (EC50: 0.5 nM, similar to cetuximab) (FIG. 5, Panel B). Similar binding patterns were observed for in vivo biotinylated VHH monomers (data not shown), and no binding of either the VHHs or VHH-Fcs was observed to EGFR-low MCF7 cells (FIG. 5, Panel C). Binding by biparatopic heterodimeric VHH/VHH-Fcs combining NRC-sdAb032 with each of the other possible VHHs was similar to the parental homodimeric NRC-sdAb032-Fc (FIG. 5, Panel D), suggesting that the monovalent interaction of the NRC-sdAb032 VHH arm with EGFR drove the majority of binding in this assay. Thus, almost all of the VHHs raised by DNA immunization recognized native EGFR on tumor cells better than EG2 VHH, and one VHH-Fc (NRC-sdAb032-Fc) had an EC50 1-2 logs lower than previously reported VHHs (in bivalent form) raised by recombinant protein immunization [4,6].

NRC-sdAb032, a VHH Elicited by DNA Immunization, Inhibited EGFR Signaling with Potency Similar to Cetuximab

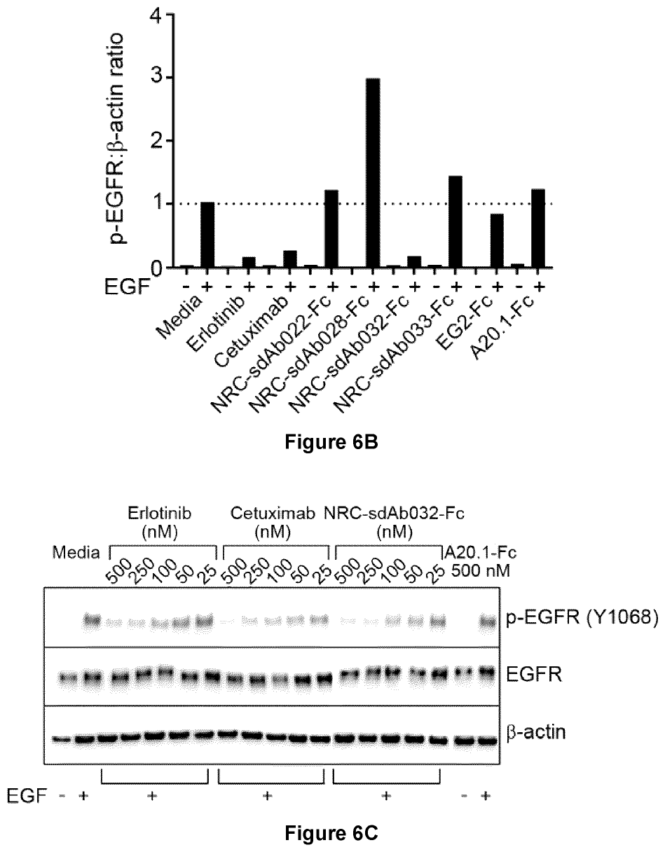

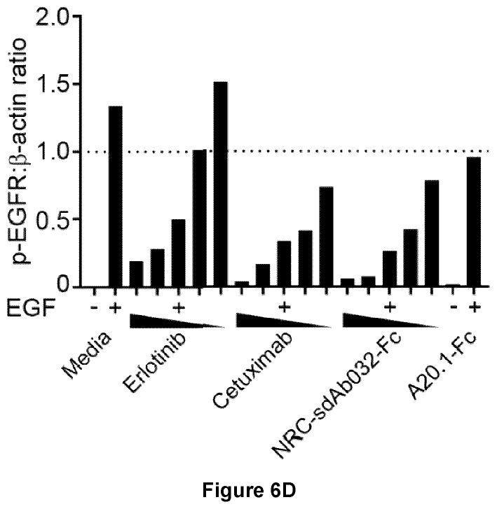

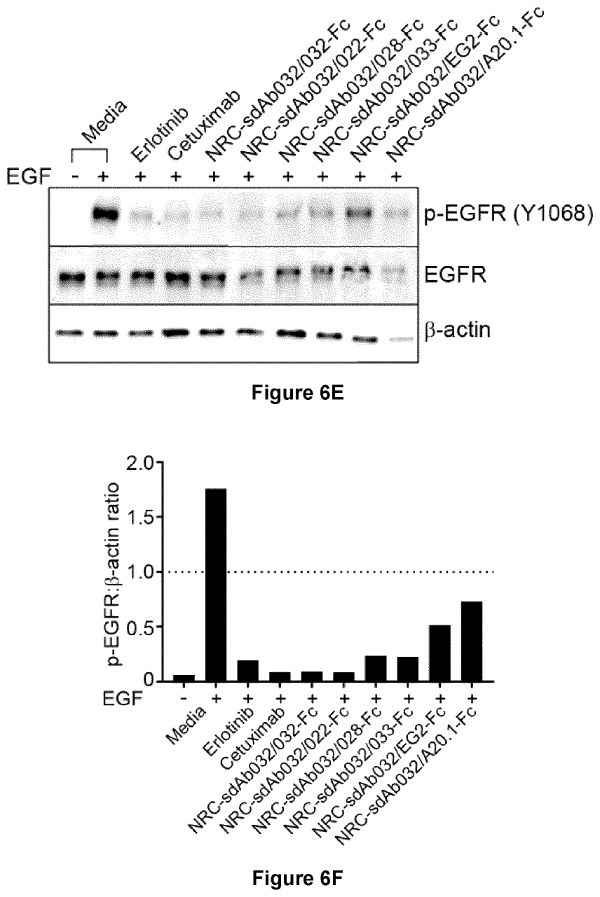

The ability of all of the VHH-Fcs showing significant binding to EGFR-positive tumor cells (NRC-sdAb022-Fc, NRC-sdAb028-Fc, NRC-sdAb032-Fc and NRC-sdAb033-Fc), as well as cetuximab and EG2-Fc as a historical control, to inhibit EGF-induced EGFR phosphorylation was tested. At a single concentration of 500 nM, NRC-sdAb032-Fc was the only VHH-Fc showing significant inhibition of EGFR signaling (FIGS. 6A, 6B). Reduction in EGFR phosphorylation in the presence of NRC-sdAb032-Fc was dose-dependent, with an IC50 between 25 and 50 nM, similar to that of cetuximab (FIGS. 6C, 6D). No improvement in potency was achieved by biparatopic heterodimeric VHH/VHH-Fcs combining NRC-sdAb032 with each other VHH, although there appeared to be a general advantage of molecules bearing two EGFR-binding arms (other than EG2) over VHH/VHH-Fcs bearing a single EGFR-binding arm (FIGS. 6E, 6F).

Discussion

The VHHs isolated and characterized here were generated by immunization of a llama with DNA alone (no protein or cell boost), using biolistic transfection (gene gun) followed by intradermal injection (DERMOJET). Many of the VHHs had high affinities for recombinant human EGFR, cross-reacted with rhesus and/or mouse EGFR, and recognized native cell-surface EGFR on tumor cell lines; in all of these respects, the VHHs described here are dramatically superior to those previously isolated by our group using recombinant protein immunization [4]. One VHH, NRC-sdAb032, showed clear functional activity as an inhibitor of EGFR signaling, and any of the humanized VHHs may have other therapeutic applications. NRC-sdAb032 was isolated by panning on streptavidin-captured EGFR but not on passively adsorbed EGFR, and its epitope appears to be present on native EGFR and recombinant EGFR ectodomain in solution but not in the same ectodomain when it is passively adsorbed to microtiter plates or amine-coupled to sensor chips. This epitope, which overlaps the cetuximab epitope and the EGF-binding site in EGFR domain Ill, is apparently poorly available for binding by NRC-sdAb032 or cetuximab in ELISAs against directly adsorbed EGFR or in SPR experiments in which these antibodies are flowed over amine-coupled EGFR surfaces; in contrast, both NRC-sdAb032 and cetuximab bound EGFR⁺ tumor cells with lower _{EC50s} than other VHHs with similar monovalent binding affinities for recombinant EGFR. It appears that the NRC-sdAb032 epitope, though easily destroyed, contributes to this VHH's superior recognition of native EGFR displayed on the tumor cell surface.

### Example 2

Humanization of V_{H}Hs

With the aim of using these VHHs in therapeutic applications, the sequences of the four VHHs showing significant binding to EGFR-positive tumor cells (NRC-sdAb022, NRC-sdAb028, NRC-sdAb032 and NRC-sdAb033) were humanized with reference to human IGHV3-30*01 and IGHJ1*01 germline genes. Humanization and testing can be accomplished using routine techniques. This process yielded at least one humanized variant with unimpaired solubility and EGFR-binding affinity for each of three parent VHHs (NRC-sdAb028-H1, NRC-sdAb032-H1 and NRC-sdAb033-H2). The framework regions of these humanized variants bore 89-94% sequence identity with human IGHV3-30*01, with no or minimal impact on the biophysical properties and EGFR-binding affinities of the resulting VHHs (Tables 4 and 5).

TABLE 4
Aggregation propensities and monovalent affinities
of humanized VHHs for human EGFR-Fc

| | Monomer | | | |
|---|---|---|---|---|
| V_{H}H | (%)¹ | kₒₙ (M⁻¹ s⁻¹) | k_{off} (s⁻¹) | K_{D} (nM) |
| NRC-sdAb022 | >95 | 2.5 × 10⁵ | 2.2 × 10⁻³ | 8.9 ± 0.1 |
| NRC-sdAb022-H1 | n.d.² | | | |
| NRC-sdAb022-H2 | <50 | n.b.³ | n.b.³ | n.b.³ |
| NRC-sdAb022-H3 | n.d.² | | | |
| NRC-sdAb028 | >95 | 9.2 × 10⁴ | 5.5 × 10⁻⁴ | 6.0 ± 0.1 |
| NRC-sdAb028-H1 | >95 | 9.3 × 10⁴ | 5.8 × 10⁻⁴ | 6.2 ± 0.2 |
| NRC-sdAb028-H2 | n.d.² | | | |
| NRC-sdAb028-H3 | n.d.² | | | |
| NRC-sdAb032 | >95 | 3.6 × 10⁵ | 2.5 × 10⁻³ | 6.9 ± 0.02 |
| NRC-sdAb032-H1 | >95 | 6.2 × 10⁵ | 1.0 × 10⁻² | 16.6 ± 0.5 |
| NRC-sdAb032-H2 | <50 | n.d.⁴ | n.d.⁴ | 254 ± 31 |
| NRC-sdAb032-H3 | n.d.² | | | |
| NRC-sdAb033 | >95 | 9.0 × 10⁴ | 1.6 × 10⁻⁴ | 1.8 ± 0.1 |
| NRC-sdAb033-H1 | >95 | 1.3 × 10⁵ | 2.2 × 10⁻⁴ | 1.7 ± 0.03 |
| NRC-sdAb033-H2 | >95 | 2.0 × 10⁵ | 2.6 × 10⁻⁴ | 1.4 ± 0.1 |
| NRC-sdAb033-H3 | <50 | 1.3 × 10⁵ | 1.1 × 10⁻² | 83.8 ± 3.7 |

¹Determined by SEC (% monomer peak area).
²n.d., not determined due to low or no expression.
³n.b., no binding of the purified monomeric sdAb to human EGFR-Fc could be detected.
⁴n.d., not determined because steady-state K_{D} was calculated.

TABLE 5
Sequence characteristics of humanized anti-EGFR VHHs

| | No. of amino | Overall amino | Framework region |
|---|---|---|---|
| | acid changes | acid identity | amino acid identity |
| | with respect | with human | with human |
| V_{H}H | to parent | IGHV3-30 (%) | IGHV3-30 (%)^{a} |
| NRC-sdAb022 | 0 | 71.3 | 77.5 |
| NRC-sdAb022-H1 | 12 | 83.0 | 91.3 |
| NRC-sdAb022-H2 | 14 | 85.1 | 93.8 |
| NRC-sdAb022-H3 | 17 | 88.3 | 97.5 |
| NRC-sdAb028 | 0 | 72.6 | 76.3 |
| NRC-sdAb028-H1 | 10 | 82.1 | 88.8 |
| NRC-sdAb028-H2 | 13 | 85.3 | 92.5 |
| NRC-sdAb028-H3 | 17 | 89.5 | 97.5 |
| NRC-sdAb032 | 0 | 67.4 | 75.0 |
| NRC-sdAb032-H1 | 13 | 80.0 | 91.3 |
| NRC-sdAb032-H2 | 16 | 83.2 | 95.0 |
| NRC-sdAb032-H3 | 18 | 85.3 | 97.5 |
| NRC-sdAb033 | 0 | 67.4 | 75.0 |
| NRC-sdAb033-H1 | 12 | 79.0 | 90.0 |
| NRC-sdAb033-H2 | 15 | 82.1 | 93.8 |
| NRC-sdAb033-H3 | 18 | 85.3 | 97.5 |

^{a}IMGT framework definitions

### Example 3

Generation of Chimeric Antigen Receptor Constructs

Overview

Chimeric antigen receptor (CAR) technology has revolutionized the treatment of B-cell malignancies and steady progress is being made towards CAR-immunotherapies for solid tumours. In the context of CARs targeting antigens which are commonly overexpressed in cancer but also expressed at lower levels in normal tissues, such as epidermal growth factor family receptors EGFR or HER2, it is imperative that any targeting strategy consider the potential for on-target off-tumour toxicity. Molecular optimization of the various protein domains of CARs can be used to increase the tumour selectivity.

Herein, high-throughput CAR screening is used to identify a novel camelid single-domain antibody CAR (sdCAR) targeting human epidermal growth factor (EGFR) with high EGFR-specific activity. To further optimize the target selectivity of this EGFR-sdCAR, progressive N-terminal single amino acid truncations of an extended human CD8 hinge domain [(G4S)₃GG-45CD8h] were made tested to improve selectivity for EGFR-overexpressing cells. Varying hinge domains in scFv-based CARs targeting EGFR-family tumour associated antigens EGFRvII and HER2 were compared.

Through comparison of various hinge-truncated scFv- and sdAb-based CARs, it is shown that the CAR hinge/spacer domain plays varying roles in modifying CAR signaling depending upon target epitope location. For membrane-proximal epitopes, hinge truncation by even a single amino acid resulted in fine control of CAR signaling strength. Hinge-modified CARs showed consistent and predictable signaling in Jurkat-CAR cells and primary human CAR-T cells in vitro and in vivo. Overall, these results indicate that membrane-proximal epitope targeting CARs can be optimized through hinge length tuning for improved target selectivity and therapeutic function.

Materials and Methods

CAR cloning

Three of the above-described EGFR-specific sdAb sequences were cloned into a modular CAR backbone using PCR amplification and single-pot restriction ligation as previously described[47]. EGFR-sdCAR constructs bearing either a full-length human 45 amino acid CD8 hinge (45CD8h) or progressively N-terminally truncated hinge variants (34CD8h, 22CD8h, or no hinge) were cloned using Gibson assembly. A library of sdAb021-CAR truncation mutants with single amino acid N terminal truncations of the human CD8 hinge extended with an additional N-terminal flexible linker [(GGGGS)₃GG-CD8h] was generated using a modular hinge-CAR with convenient type-IIs restriction sites integrated into the construct 3′ of the sdAb coding region. An array of DNA encoding truncated CD8 hinge domains of varying lengths (60 to 1 amino acid) was synthesized as DNA fragments (Twist Bioscience, USA) and cloned into the sdAb021-modular-hinge-BBz-GFP CAR construct using single-pot restriction ligation. Limited hinge truncation libraries with defined-target CARs were generated by exchanging the sdAb021 sequence with appropriate scFv sequences. Trastuzumab derived scFv sequences were generated based on previously reported mutant forms of trastuzumab with enhanced avidity, whereas EGFRvIII-targeting scFvs were generated as previously reported[47]. Both HER2- and EGFRvIII-scFvs were in a VH-(G4S)₃-VL format.

Cell Lines and Culture

All cell lines were purchased from American Tissue Culture Collection (ATCC, Manassas, VA). The glioblastoma cell line U97MG-WT and U87MG-vIII (U87-vIII, expressing EGFRvIII via retroviral transduction and sorting) were kindly provided by Professor Cavnee, from the Ludwig Institute for Cancer Research, University of California, San Diego (San Diego, CA, USA). Cell lines used were Jurkat, and target cells SKOV-3, MCF-7, U-87-MG vll, Raji, and Nalm6. Target cells were transduced with lentivirus containing NucLight Red (Sartorius, Essen BioScience, USA), a third generation HIV-based, VSV-G pseudotyped lentivirus encoding a nuclear-localized mKate2. All cell lines were cultured in RPMI supplemented with 10% fetal calf serum and 1% penicillin/streptomycin. These cell lines were tested for the presence of mycoplasma contamination by PCR.

CAR-J Assay

Jurkat cells were transfected via electroporation according to a previously outlined protocol[47]. Briefly, 5×10\hat{ }5 cells were suspended in 100 μl Buffer 1SM (5 mM KCl, 15 mM MgCl₂, 120 mM Na₂HPO₄/NaH₂PO₄, 25 mM sodium succinate, and 25 mM mannitol; pH7.2) and incubated with 2 μg of pSLCAR-CAR plasmids as described in the text or with no plasmid control. Cells and plasmid DNA in solution were transferred into 0.2 cm generic electroporation cuvettes (Biorad Gene Pulser; Bio-Rad Laboratories, Hercules, California, USA) and immediately electroporated using a Lonza Nucleofector I (Lonza, Basel, Switzerland) and program X-05 (X-005 on newer Nucleofector models). Cells were cultured in pre-warmed recovery media (RPMI containing 20% FBS, 1 mM sodium pyruvate and 2 mM L-glutamine) for four 4h before being co-cultured with EGFR-expressing target cells U-87MG-vIII, MCF7 and SC-OV-3 or negative control Ramos and Nalm6. Electroporated Jurkat cells were added to varying numbers of target cells in round bottom 96-well plates in effector to target (E:T) ratios ranging from 1:10 to 100:1 (effector to target ratio) or with no target cells (or an E:T of 1:0) and cultured overnight before being staining with allophycocyanin (APC)-conjugated anti human-CD69 antibody (BD Biosciences #555533). Flow cytometry was performed using a BD-Fortessa (BD Biosciences) and data was analyzed using Flowjo software (Flowjo LLC, Ashland, Oregon, USA) and visualized using GraphPad Prism (GraphPad Software, Inc. California, USA).

Human Peripheral Blood Mononuclear Cell (PBMC) Isolation

Heparinized whole blood was collected from healthy donors by venipuncture and transported at room temperature from Ottawa Hospital Research Institute. Blood was diluted 1:1 with Hank's balanced salt solution (HBSS) and PBMCs were isolated by Ficoll density gradient centrifugation. Briefly, samples layered on Ficoll gradient were centrifuged for 20 min at 700 x g without applying a brake. The PBMC interface was carefully removed by pipetting and was washed twice with HBSS by stepwise centrifugation for 15 min at 300 x g. PBMC were resuspended and counted by mixed 1:1 with Cellometer ViaStain™ acridine orange/propidium iodide (AOPI) staining solution and counted using a Nexcelom Cellometer Auto 2000 (Nexcelom BioScience, Lawrence, Massachusetts, USA).

T cells from freshly isolated PBMCs were activated with Miltenyi MACS GMP TransAct CD3/CD28 beads and seeded at 1×10⁶ cells/ml in serum-free StemCell Immunocult-XF media (StemCell Technologies, Vancouver, Canada) containing clinical grade human IL-2 (20 U/ml, Novartis) or Miltenyi TexMACS containing research grade human IL-7 and human IL-15 (both 10 ng/ml).

T cells from freshly isolated PBMC were activated with Miltenyi MACS GMP TransAct CD3/CD28 beads and seeded 1×10⁶ T cells/ml in serum-free StemCell Immunocult-XF media (StemCell Technologies, Vancouver, Canada) with clinical grade 20 U/ml human IL-2 (Novartis) or Miltenyi TexMACS with research grade 10 ng/ml human IL-7 and 10 ng/ml human IL-15.

Human Primary T Transduction by Spinfection

High concentration lentiviral particles encoding various sdCAR constructs were generated as previously described [47]. After 24 h of T cell stimulation with beads, T cells were transduced with sdCAR-GFP lentiviral vectors (multiplicity of infection=10) by spinfection. Briefly, lentivirus was added to T cells (1×10⁶ cells/ml) and the mixture was centrifuged at 850×g for 2 h at 32° C. After centrifugation, cells were incubated at 37° C. for another 2 h. After incubation, cells were plated in a 24 well plate (100,000 cells/ml/well in a total of 1.5 mL) in media supplemented with cytokine(s). Media with cytokine(s) was added at 48 and 72 h post transduction to promote CAR-T cell proliferation without disrupting the cells. CAR-T cells were sampled daily until the end of production. Cell number and viability were assessed by AOPI staining and counting using a Nexcelom Cellometer. CAR-T cells were propagated until harvest on days 7, 9, 14, and 21 to assess the efficiency of transduction and to characterize T cell subpopulations by flow cytometry. CAR-T cells that had returned to a resting state (as determined by decreased growth kinetics, day 10 post-T cell activation) were used for assays. Expression of GFP-CARs by transduced T cells ranged from 20% to 70%.

Continuous Live-Cell Imaging Cytotoxicity Assay

Cytotoxicity of the CAR-T cells was assayed using a Sartorius IncuCyte S3 (Essen Bioscience). Tumour cells (U87vIII-NucLight, MCF7-NucLight, and SKOV3-NucLight were plated in a flat bottom 96-well plate (2000 cells/well). CAR-T cells or control T cells were added into each well in a final volume of 200 μl per well at varying effector:target ratios and co-cultured for 7 days at 37° C. Images were taken every 30 min in light phase and under red (ex. 565-605 nm; em. 625-705 nm) or green fluorescence (ex. 440-480 nm; em. 504-544 nm). The assays were repeated twice with T cells derived from independent blood donors. For one donor, CAR-T cells challenged once or twice with EGFR-high SKOV3 cells were rechallenged with various freshly plated target cells after 7 day of co-culture. Automated cell counting of red (target) or green (CAR-T) cells was performed using Incucyte analysis software and data were graphed using GraphPad Prism.

Animal Studies

NOD/SCID/IL2Ry^{-/-} (NSG, JAX #005557) mice were purchased from Jackson Laboratories and maintained by the Animal Resource Group at the National Research Council of Canada. Eight-week-old NSG mice were injected with 2×10⁶ SKOV-3 in 100 μL of PBS subcutaneously. Eighteen days post tumour cells injection (when tumour reached 5 mm×5 mm), mice were retro orbitally injected with 5×10⁶ mock T cells or T cells transduced with various CAR-T cells as described in the text. Tumours were measured using calipers twice a week and mice were imaged via IVS in vivo imager for red-fluorescence signal (expressed on tumour cells) once a week. For the alternative U87vIII model experiments, mice were subcutaneously injected with 1×10⁶ fluorescently labelled U87-vIII cells described above, a number previously determined to consistently produce a palpable tumour within 7 days. Eight days after tumour cell injection, cryo-preserved CAR-T cells were thawed, washed with PBS, and 1×10⁷ total T cells (with 20-25% CAR transduction) were immediately delivered intra-tumourally, ensuring equal distribution of tumour sizes between groups. Tumour growth was evaluated three times per week using calipers by trained animal technicians blinded to specific treatment groups. Primary endpoint was tumour size above 2000 mm³, with secondary endpoints determined by overall animal health and well-being. Mice were also assessed for tumour growth using IVIS in vivo imaging to examine red fluorescence derived from the NLS-mKate2 marked U87vIII cells. Mice were euthanized when they met pre-specified endpoints. The study was approved by the NRC-HHT Institutional Animal Care Committee and was conducted in accordance with Canadian Council on Animal Care (CCAC) guidelines. Tumour growth and survival (humane endpoint) curves were generated using GraphPad Prism.

Flow Cytometry and Antibodies

Blood was obtained from mice at various time points post CAR-T injection. Blood was washed with cold PBS and pelleted at 350×g for 5 min at 4° C. Red blood cells were lysed using Red Blood Cell Lysing Buffer Hybri-Max (Sigma-Aldrich, St. Louis, MO, USA). Human T cells were identified and analyzed for activation/differentiation status using the following antibodies: hCD45-APC-H7, hCD45RA-BV650, hCD45RO-PE-CF594, hCD27-BUV737, hCCR7-PE, hCD4-BUV395, and hCD8-PerCP-Cy5.5 (all antibodies from BD Biosciences, USA). CAR expression was measured indirectly via expression of GFP incorporated in CAR constructs. To evaluate exhaustion, staining by an hPD-1-BV421 antibody was evaluated. T cell activation was detected using hCD25-PE-Cy7 and hCD69-BV786 antibodies. For in vivo studies, a BV71 1-labeled antibody against mouse CD45 was used to identify murine cells and CD19 expression was analyzed using an anti-human CD19-BUV496 antibody. Staining of human EGFR was performed using anti-human EGFR-PE-CF594 (BD Biosciences, Cat #563431).

Results

High-Affinity EGFR-Specific sdAbs Generate Antigen-Responsive CARs with Low Tonic Signaling

Camelid sdAbs were raised in a previous study against EGFR using DNA immunization and phage display. In order to assess whether these sdAbs were functional in the context of a sdCAR three EGFR-specific sdAbs with varying affinities as discussed above and epitopes were selected (Table 6).

TABLE 6
Antigen-binding domains tested as CAR elements in this study

| Antibody | | | Affinity | Cross- | | Epitope | |
|---|---|---|---|---|---|---|---|
| Name | Type | Target | (K_{D}) | Reactivity | Epitope | Location | Ref |
| sdAb021 | sdAb | EGFR | 38.5 nM | Human, | A; partial | Unknown | |
| | | (Erbb1) | | Cyno | overlap with B | | |
| sdAb027 | sdAb | EGFR | 1.6 nM | Human, | A; partial | Unknown | |
| | | (Erbb1) | | Cyno | overlap with B | | |
| sdAb028 | sdAb | EGFR | 9.0 nM | Human, | B; partial | Unknown | |
| | | (Erbb1) | | Mouse | overlap with A | | |
| Trastuzumab | scFv | HER2 | n.d. | Human, | Juxtamembrane | Very | [51, 58] |
| | | (Erbb2) | (5 nM)^{a} | Cyno | conformational | membrane | |
| | | | | | epitope in | proximal | |
| | | | | | domain IV | | |
| F265 | scFv | EGFRvIII | n.d. | Unknown | Domain I/III | Membrane | [47] and |
| | | | (27.5 nM)^{a} | | neoepitope, | distal | unpublished |
| | | | | | non-competitive | | |
| | | | | | with F269 | | |
| F269 | scFv | EGFRvIII | n.d. | Unknown | Domain I/III | Membrane | [47] and |
| | | | (31.5 nM)^{a} | | neoepitope of | distal | unpublished |
| | | | | | EGFR, non- | | |
| | | | | | competitive | | |
| | | | | | with F265 | | |

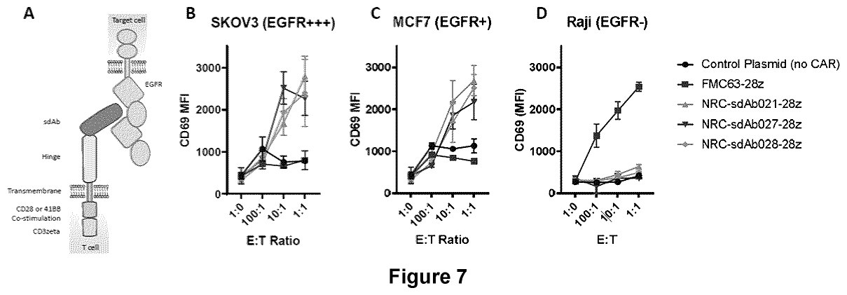

The sdAbs were cloned into a modular CAR backbone and the resulting sdCARs were screened for responses to target cells with varying EGFR expression using a previously described high throughput Jurkat screening assay[47] (FIG. 7, Panel A). Jurkat cells electroporated with any of three EGFR-specific sdCAR constructs showed specific upregulation of 0069 following co-culture with EGFR-high SKOV3 cells or EGFR-low MCF7 cells (FIG. 7, Panels B and C). In contrast, Jurkat cells expressing EGFR sdCARs showed no activation in response to EGFR-negative Raji cells (FIG. 7, Panel 0). These data confirmed that high affinity EGFR sdAbs can effectively redirect CAR signaling towards EGFR-expressing cell lines.

Hinge Shortening Decreases CAR Signaling

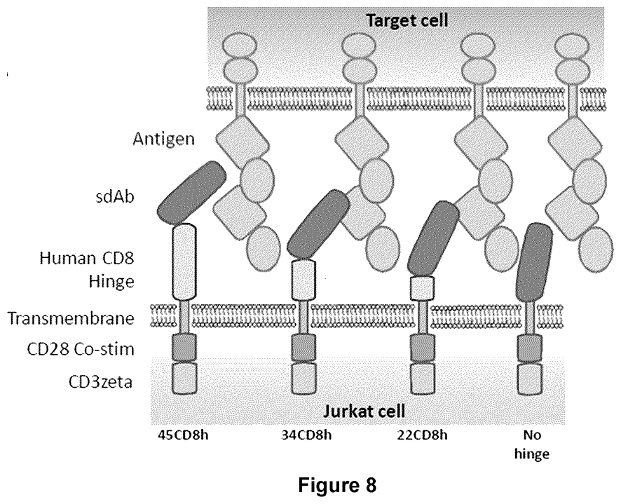

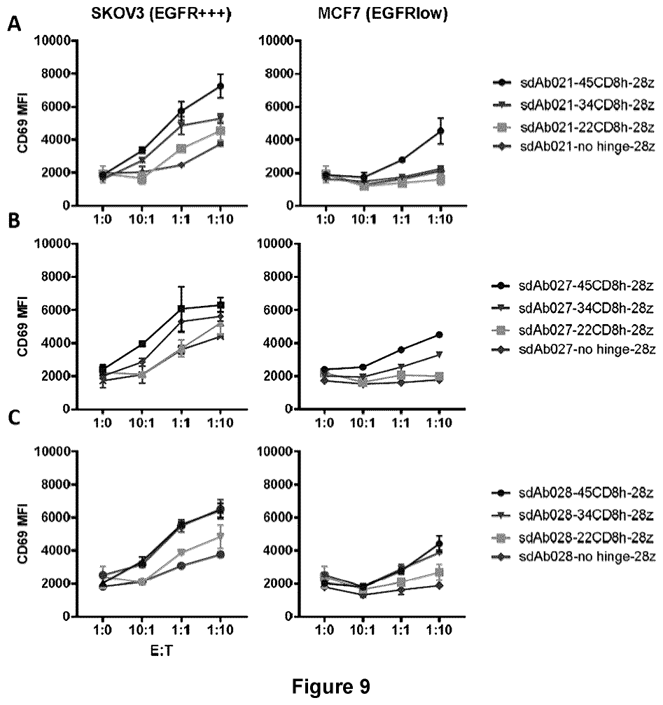

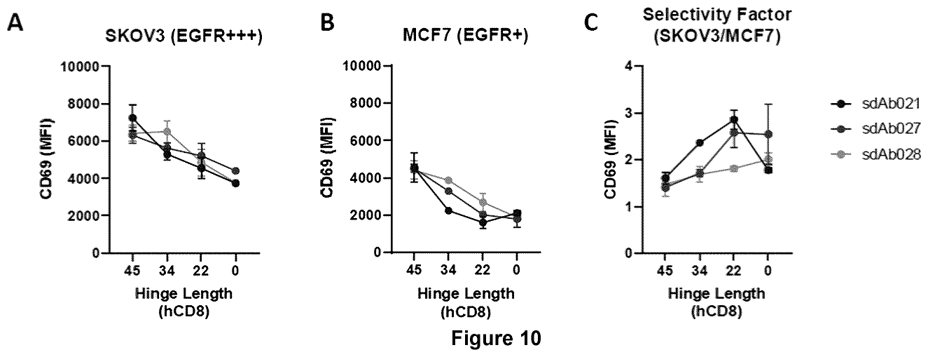

It has previously been shown that altering the length of the spacer (hinge) between the antigen binding domain (ABD) and transmembrane domain can be used to modulate CAR signaling[48,49]. It was next investigated whether the selectivity of the EGFR sdCARs for EGFR-high cells could be increased by progressively decreasing the length of the hinge region. EGFR sdCAR constructs were cloned with either a full-length 45 amino acid human CD8 hinge (45CD8h) or progressively N-terminally truncated hinge variants (34CD8h, 22CD8h, or no hinge) (FIG. 8). Jurkat cells transiently expressing the truncated hinge variants of all three EGFR sdCARs showed progressively decreasing activation in response to EGFR-high SKOV3 cells (FIG. 9, Panel A to C). Intriguingly, EGFR sdCAR responses to EGFR-low MCF7 cells appeared to be more sensitive to hinge truncation, particularly for the sdAb021 sdCAR (FIG. 9, Panel A to C, and FIG. 10).

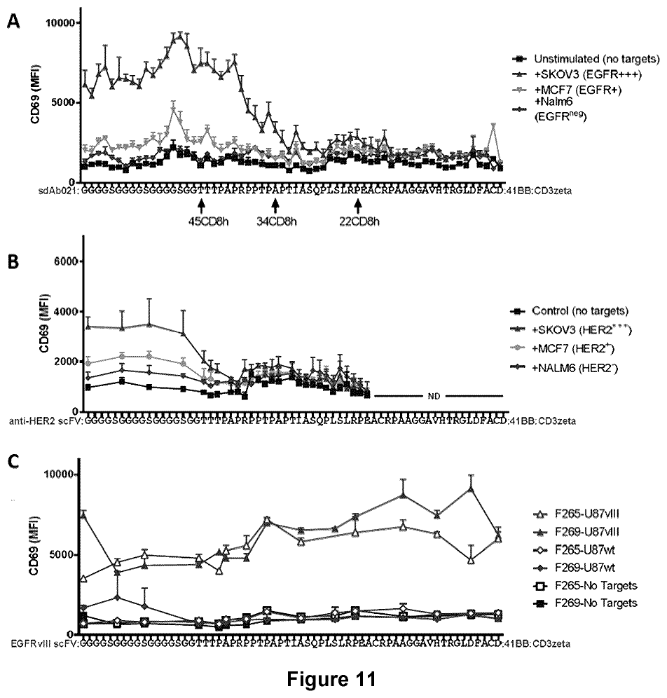

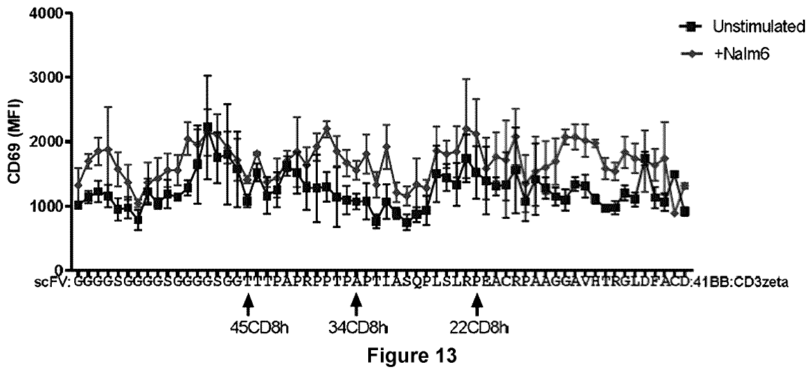

Experiments were designed to more finely map the effects of hinge length modulation on an EGFR sdAb CAR. As a starting point, an extended hinge domain was designed wherein an additional N-terminal flexible linker of 17 amino acids was included before the human CD8-hinge sequence [(GGGGS)₃GG-CD8h]. A library of sdAb021-CAR constructs with single amino acid N-terminal deletions of the human CD8 hinge was generated. Screening the sdCAR single-residue hinge truncation library revealed a clear pattern of CAR activation (FIG. 11, Panel A). CAR constructs containing a hinge domain of a full human CD8-hinge or longer produced a consistently high response to SKOV3 cells. Interestingly, addition of a short SGG N-terminal linker slightly increased CAR signaling against EGFR-high SKOV3 and EGFR-low MCF7 cells. CARs with CD8 hinge sizes between 39 and 32 amino acids showed a progressive decrease in CAR activation, while CAR constructs with hinges less than 26 residues showed no apparent difference in CAR-specific response between EGFR-high target and EGFR-negative controls. As in experiments above, it appeared that CAR activation fell more quickly in MCF7 cells relative to SKOV3, providing a range of CAR constructs wherein response to SKOV3 remains but MCF7-response is not significantly different from response to irrelevant target cells (NALM6). Data was re-examined, isolating tonic (target-independent) signaling and response to EGFR-negative NALM6 cells; no consistent trend in antigen independent activation of constructs with varying hinge length was observed (FIG. 13). Taken together, these data suggest that fine hinge mapping may be an effective strategy to optimize CAR selectivity for EGFR-overexpressing tumours.

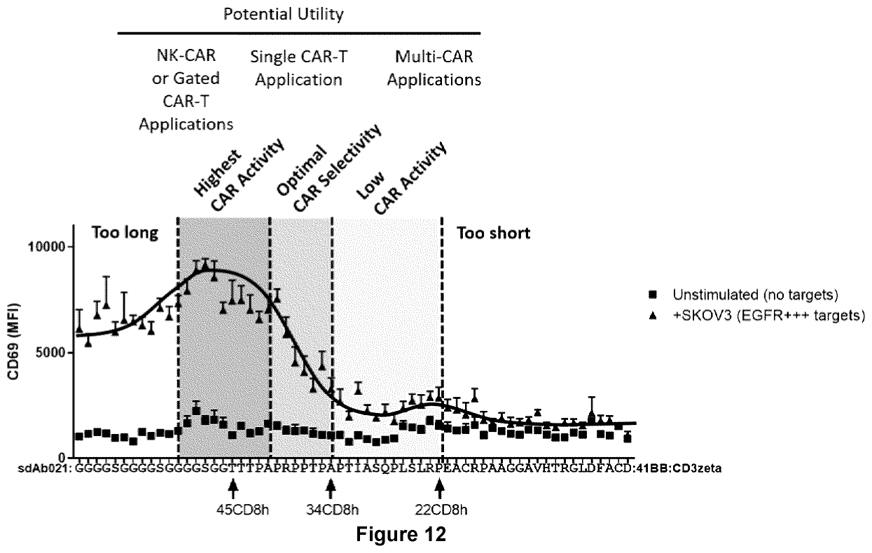

Examining the range of CAR activity across the full array of single-amino acid truncations tested, it is expected that particular ranges of hinge length may have applicability to different cellular immunotherapy targeting strategies (FIG. 12). Specifically, hinges ranging between GGGGSGG-45CD8h and 40-CD8h have high signaling activity; sdAb021 CARs constructed with this range of hinge elements would be suited to targeting strategies that incorporate additional mechanisms that can downregulate on-target off tumour responses and may include but are not limited to Natural Killer based cellular immunotherapy or Boolean-gated CAR receptors. A hinge domain containing between 40 and 34 amino acids from the CD8 hinge domain may result in a CAR that has balanced single target selectivity, yet maintains reasonable on target activity; such constructs may be ideally suited to single construct CAR applications. CAR constructs containing a hinge element between 34 and 22 amino acids of the human CD8 hinge domain induce low CAR-T activation below a threshold necessary for tumour targeting. Such constructions would not function therapeutically in the absence of additional signally, but may be best suited to multi-antigen targeting CAR applications that incorporate additional targeting elements; that may include but are not limited to tandem CAR constructs containing multiple antigen binding domains, bi-cistronic CAR constructs wherein multiple CAR receptors are encoded in a single viral construct, co-transduction of CAR viruses targeting different antigens, or co-administration of separate CAR-T product which target different antigens.

Epitope Location is a Determinant of CAR Hinge Sensitivity

As has been previously proposed, the location of the CAR target epitope should be critical in determining the minimal hinge length necessary for CAR signaling[50]. Epitope binning experiments indicated that all three sdAbs have partially overlapping epitopes, and cross-reactivity analysis is suggestive of membrane proximal domain IV binding (Table 6). To more carefully investigate the role of epitope location, limited hinge libraries were generated for CARs with known target epitopes. scFv-CARs were generated based on either trastuzumab, which is known to bind a highly membrane proximal epitope of HER2 [51], or novel EGFRvII-specific antibodies[47], which by necessity must bind the membrane distal neo-epitope of EGFRvII (Table 6). As hypothesized, the trastuzumab CAR required a very long hinge element: no HER2 scFv CARs containing shorter than a full CD8-hinge showed any response (FIG. 11, Panel B). In contrast, membrane-distal targeting EGFRvII CARs maintained full activation when the entire CD8 hinge domain was deleted (FIG. 11, Panel C). These data clearly demonstrated that epitope location is a key determinant of the hinge length required for maximal CAR signaling.

Hinge-Truncated CAR Signaling is Consistent in Primary T Cells In Vitro and In Vivo

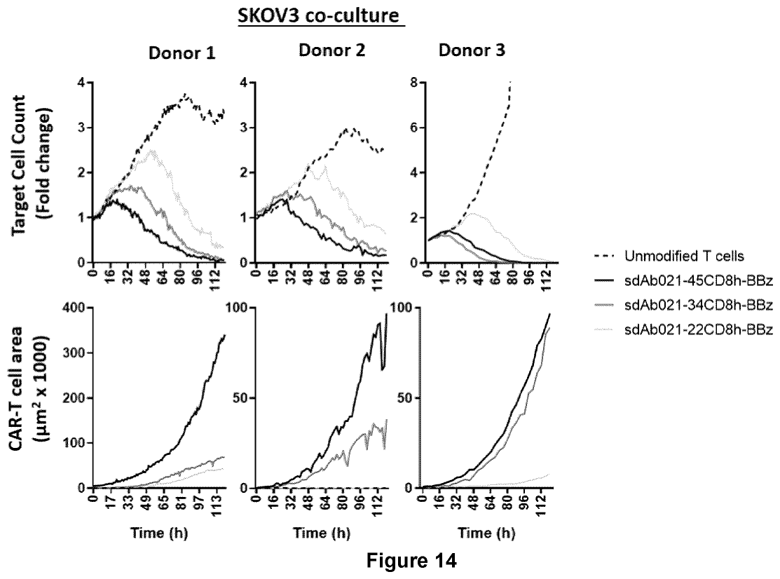

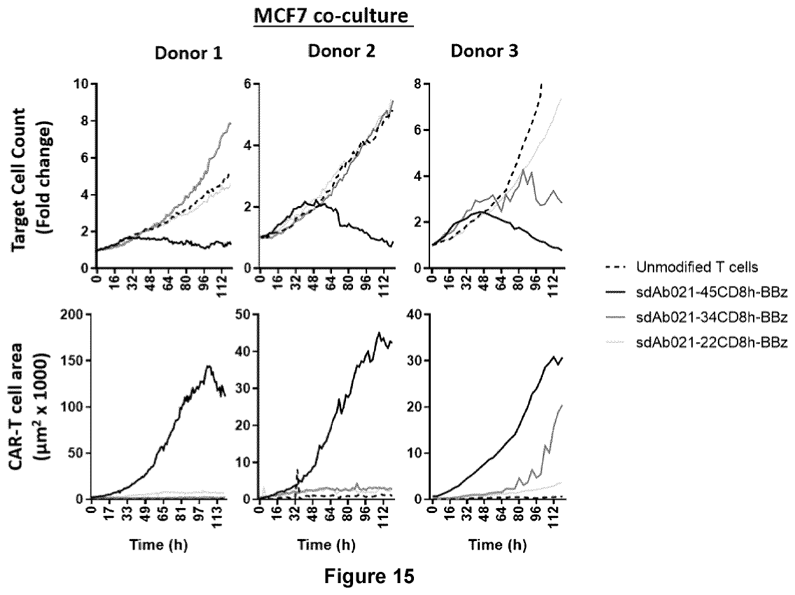

Experiments were carried out to confirm whether the reduced signaling of sdCARs with truncated hinge elements expressed in Jurkat cells was also manifested in primary CAR-T cells. Thus, T cells derived from two human blood donors were transduced with hinge-modified forms of the sdAb021 sdCAR. Following polyclonal expansion and sdCAR transduction, green fluorescent protein (GFP)+ CAR-T cells with various hinge elements were placed in low density co-culture with target cells stably marked with a nuclear localized red fluorescent protein. Co-cultures were then monitored for tumour cell (red fluorescence) and CAR-T cell (green fluorescence) expansion over 7 days. Consistent with observations in Jurkat cells, hinge truncation progressively diminished the ability of sdAb021 sdCARs to restrict tumour cell growth and expand in response to target cells (FIG. 14, FIG. 15). Also consistent with Jurkat observations, there was no obvious relationship between hinge length and CAR tonic signaling as measured by CAR-T expansion with no additional stimulation. Most importantly, although all hinge truncated sdAb-021 sdCARs were able to control expansion of EGFR-high SKOV3 cells, only the sdCAR with the unmodified hinge (sdAb021-45CD8h-28z) showed potent tumour killing and CAR-T expansion in response to EGFR-low MCF7 cells (FIG. 14, FIG. 15).

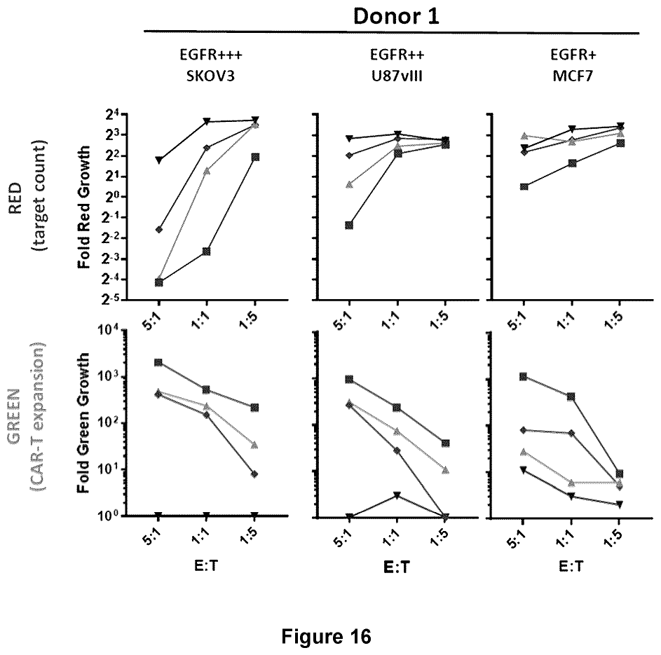

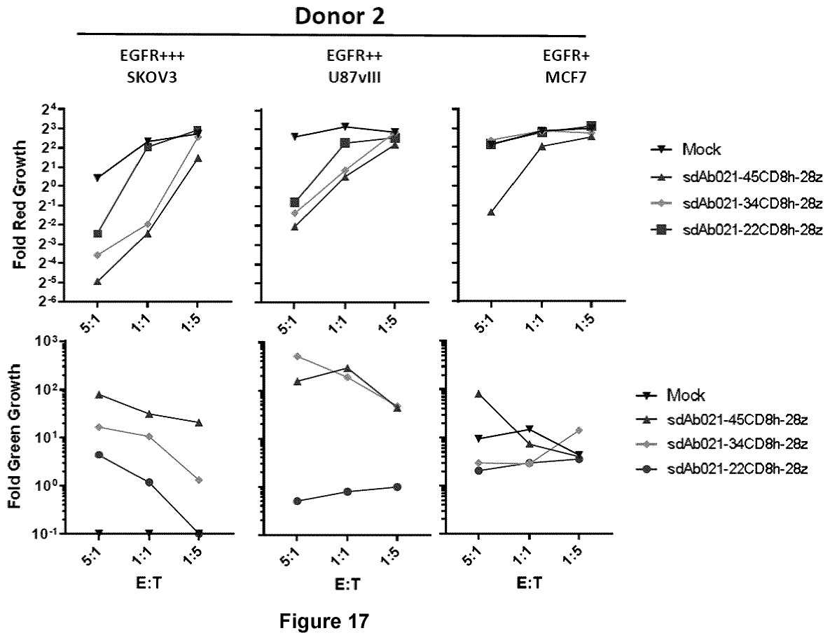

Experiments were carried out to more finely examine the effect of hinge truncation under a wider variety of antigenic conditions. Thus, hinge-modified sdAb021 sdCAR-T cells were plated with EGFR-very high (SKOV3), EGFR-medium/high (U87vIII), or EGFR-low (MCF7) target cells at varying effector:target ratios and examined them using live microscopy. Using primary T cells from both donors, sdCAR-T cells with truncated hinges maintained tumour killing and CAR-T expansion in response to EGFR-overexpressing SKOV3 and U87vIII cells but showed low responses to EGFR-low MCF7 cells (FIGS. 16 and 17). Overall, these data indicate that hinge length modulation, and specifically progressive hinge truncation, can be used to decrease responses of primary CAR-T cells against cells with varying target expression and increase selectivity for antigen overexpressing cells.

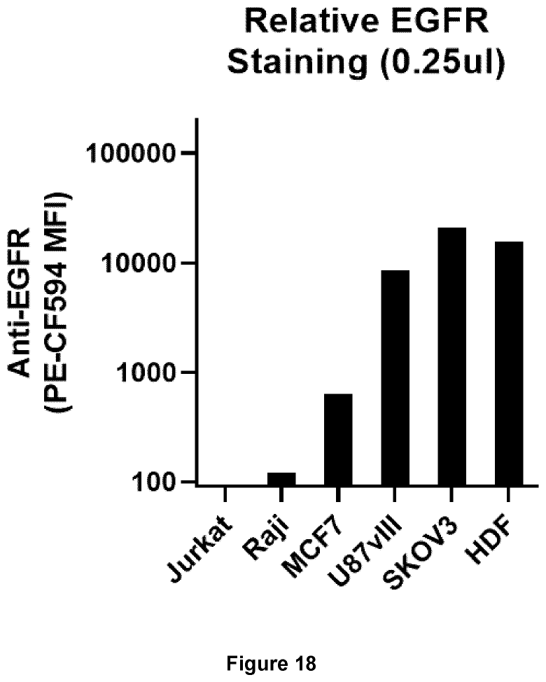

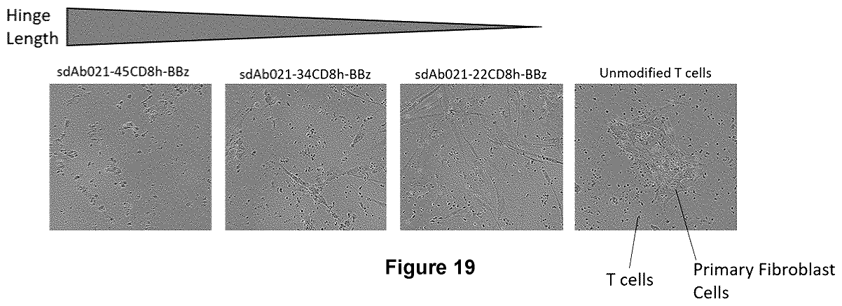

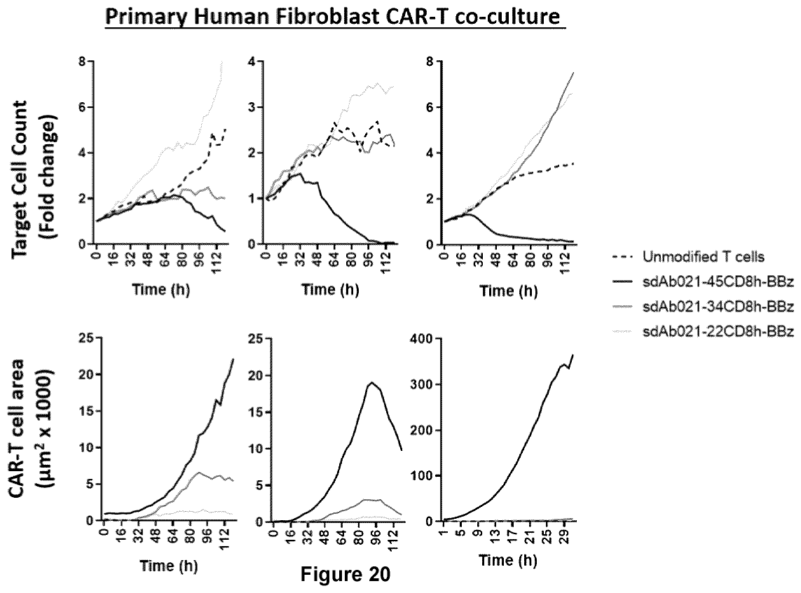

The activity of hinge-truncated CAR molecules against primary human skin fibroblast cells was also tested, which show a relatively high level of EGFR expression as measured using flow cytometry and a commercial anti-EGFR antibody (FIG. 18). Using CAR-T cells virally transduced with EGFR-targeted CAR molecules containing hinge elements of varying length, the relative response in co-cultures primary fibroblasts which were marked with a stable red-fluorescent protein was examined (FIG. 19). Clear expansion of CAR-T cells and killing of primary fibroblast cells can be observed when cells were incubated with EGFR-CAR-T cells with a full CD8-hinge domain. Similar to the observations in cancer cell lines, hinge-truncated EGFR CAR-T cells showed progressively decreasing responses against the EGFR+ healthy donor fibroblast cells (FIG. 19). FIG. depicts relative growth of primary fibroblast cells (top) and CAR-T cells (bottom) in co-cultures experiments. Healthy donor derived CAR-T cells (GFP marked) and primary fibroblast cells (RFP marked) were placed in co-culture and examined using live fluorescence microscopy (Incucyte Device, Sartiorius USA). The relative expansion of CAR-T or target cells was then assessed via automated cell counting. Results show clear killing of fibroblast cells and simultaneous expansion of CAR-T cells in EGFR-CAR-T cells with a full CD8-hinge domain, while CAR constructs with truncated hinge domains showed progressively decreasing CAR-T expansion and increasing survival of fibroblasts. These results demonstrate that hinge truncation can decrease antigen-specific responses against non-cancerous EGFR-expressing cell types, as may be present in healthy patients.

Hinge-Truncated CARs Maintain Selectivity for High-Expressing Cells Following Re-Challenge

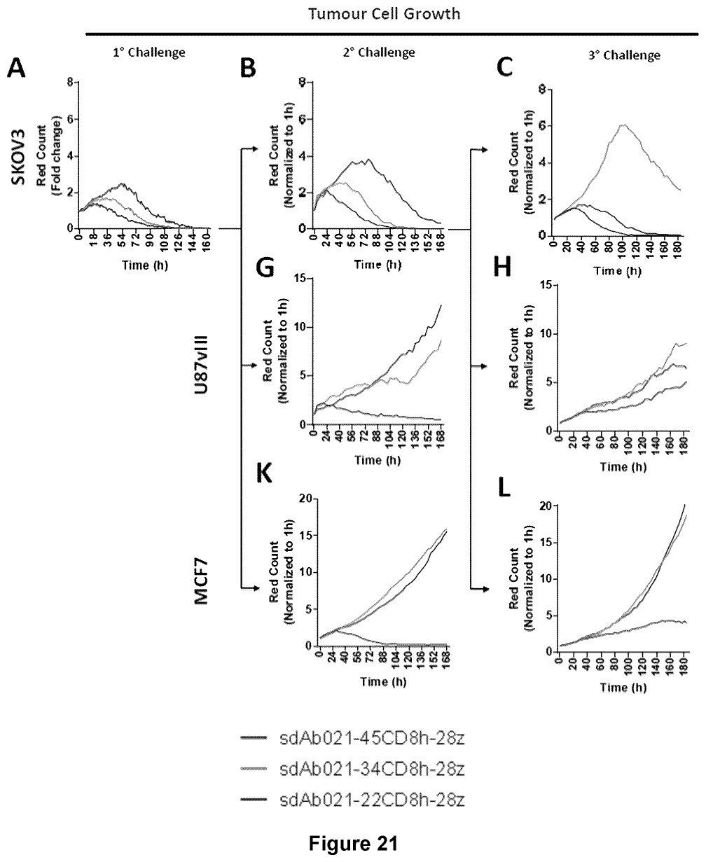

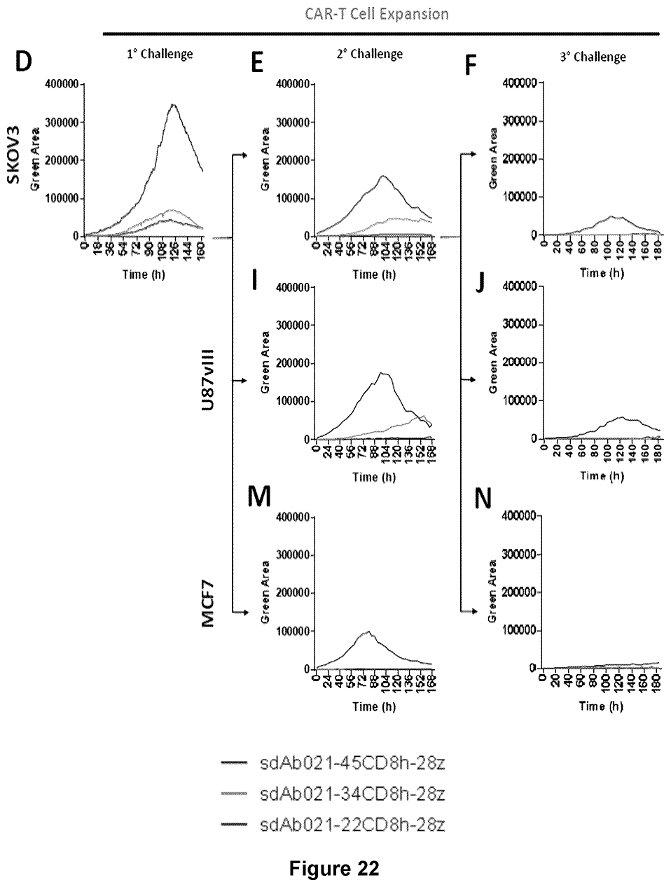

The effect of hinge truncation on serial CAR-T killing was investigated. Hinge modified sdCAR-T cells were isolated following primary challenge with antigen-overexpressing SKOV3 cells using the low density co-culture assay as described above and re-challenged the sdCAR-T cells by re-plating with additional target cells (FIG. 21, Panels A to C). Re-challenged cells maintained their relative ability to kill target cells, which also decreased with hinge length (FIG. 21, Panel B). Re-plating secondary challenge cells for a tertiary challenge revealed a similar trend in maintained tumour repression decreasing this hinge length (FIG. 21, Panel C). While target killing was mostly maintained following re-challenge, a consistent decrease in CAR-T expansion was observed in secondary and tertiary re-challenge assays (FIG. 22, Panels D to F).

To specifically address the question of whether sdCARs with truncated hinges maintained their selectivity following antigen experience, single or double SKOV3-challenged sdCAR-T cells were re-challenged with U87vIII (FIG. 21, Panels G & H; FIG. 22, Panels I & J) or MCF7 cells (FIG. 21, Panels K & L; FIG. 22, Panels M & N). Similar to SKOV3 serial challenge, re-challenged sdCAR-T cells of all hinge lengths showed decreased tumour killing and CAR-T expansion in response to both cell lines with reduced EGFR expression. These results indicated that re-stimulated CAR-T cells show similar or higher antigenic discrimination as observed in primary challenge, with progressively decreasing antigen-induced expansion following serial challenge.

In Vivo CAR-T Response is Progressively Diminished with Hinge Truncation

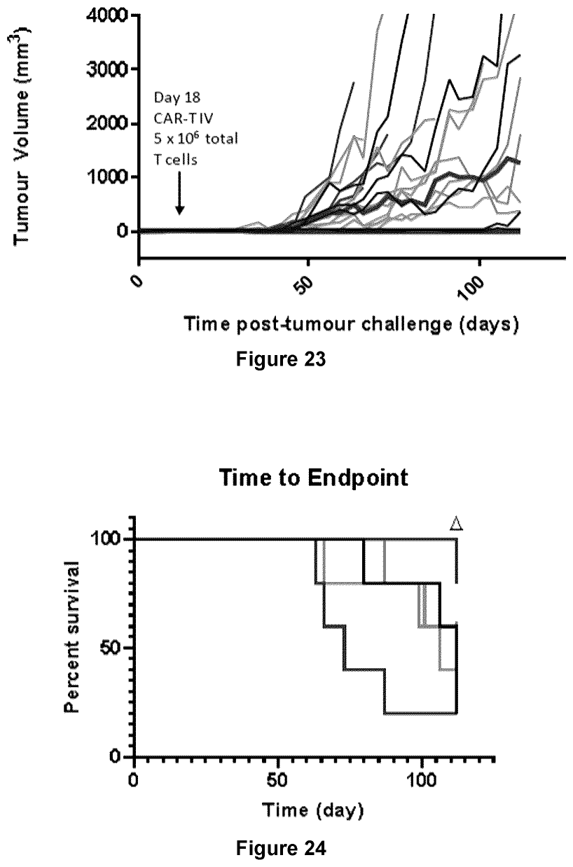

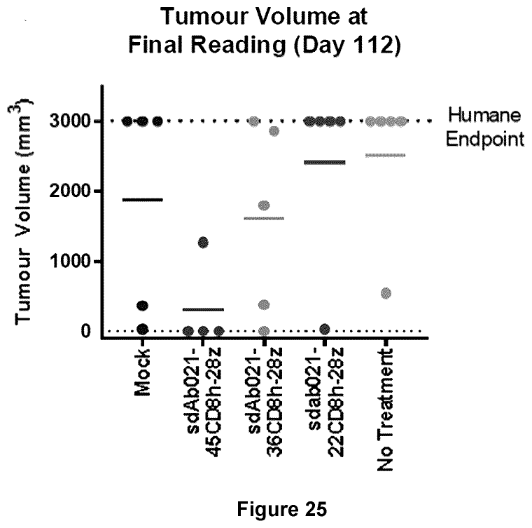

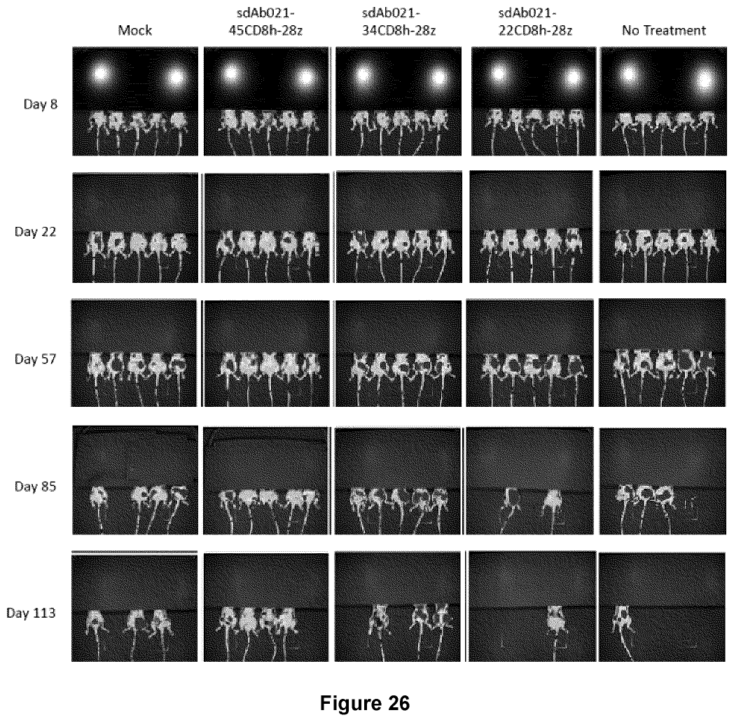

Experiments were carried out to investigate whether in vitro observations regarding the relationship between hinge length and CAR-T activity were consistent in an in vivo xenograft model. Using the relatively slow growing SKOV3 model, mice were challenged with 2 million tumour cells subcutaneously and then treated intravenously at day 18 post-tumour implantation with 5 million hinge modified sdCAR-T cells or 1.25-1.5 million mock transduced CAR-T cells. There was a progressive increase in tumour growth in all mice (FIG. 23) and decreased survival was observed in mice treated with hinge-truncated sdCAR-T cells (FIG. 24). Surprisingly, mice treated with sdCAR-T cells bearing the shortest hinge (sdAb021-22CD8h-28z) showed apparent decreased survival compared with untreated animals, although larger experiments would be needed to confirm this result. The progressive effect of hinge truncation could be clearly observed in the final tumour volume measurement taken at day 112 post tumour challenge (FIG. 25, FIG. 26) prior to early experimental termination due to non-experiment related facility disruptions.

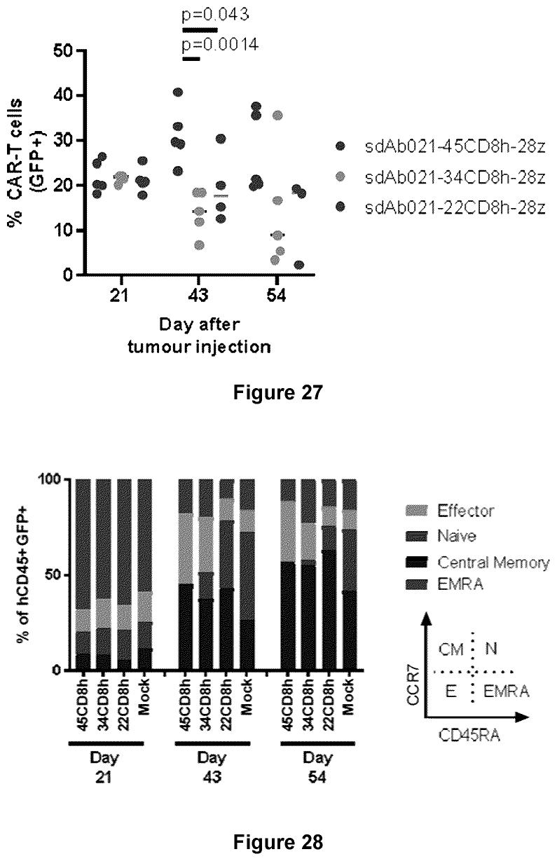

Examining CAR-T cells in the blood of treated mice revealed a consistent pattern of increased expansion of sdCAR-T cells with longer hinge regions at 43 and 54 days post-tumour injection (FIG. 27). Analysis of T cell differentiation revealed increased naïve sdCAR-T cell and decreased effector sdCAR-T cell populations were for hinge truncated sdCARs (FIG. 28), supporting the interpretation that truncated sdCAR-T cells show progressively decreased in in vivo responses. A similar experiment was performed in which wild-type or hinge modified sdCAR-T cells were delivered intratumourally in the relatively more aggressive U87vIII glioblastoma xenograft model. Similar progressive decreases in anti-tumoural effect and CAR-T expansion and progressive increases in naïve CAR-T cell populations associated with hinge truncation were observed. Taken together, these results demonstrated that hinge truncation can be used to reduce CAR-T signaling activity in vivo.

Inclusion of a Hinge Element can Increase Bi-Specific T-Cell Engaging Antibody Activity

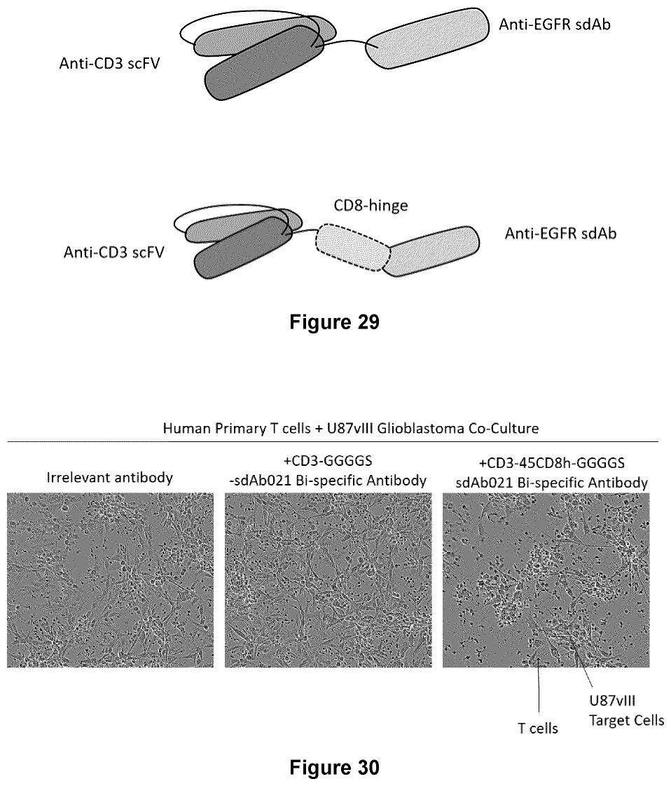

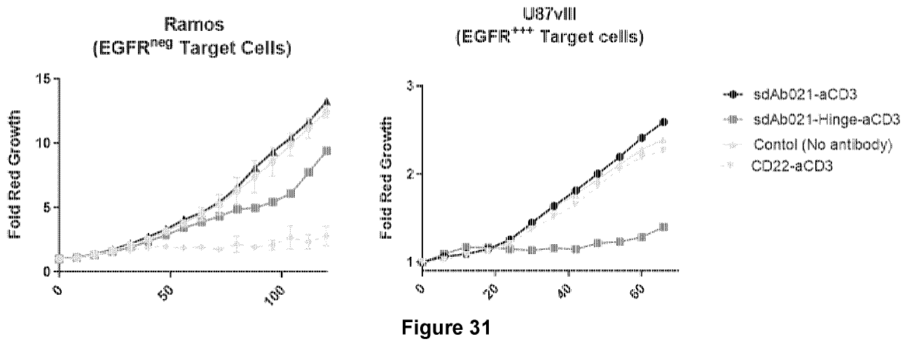

It was next assessed whether inclusion of a human CD8-hinge domain similar to that found in the CAR molecules discussed above can also increase the activity of a bi-specific antibody which can simultaneously engage both a human T-cell through the CD3 receptor and a human EGFR-expressing target cell. Thus a bi-specific antibody composed of a human CD3-specific single-chain variable fragment and an EGFR-specific sdAb021 camelid single domain antibody fragment, linked either by a short GGGGS linker alone or a long human (G4S)3-45CD8-hinge-G4S linker, were generated (FIG. 29). In brief, plasmids were generated containing corresponding sequences of bi-specific CD3-EGFR sdAb021 based antibody with a short or long hinge domain (SEQ ID: 70). Plasmid constructs were then transfected in HEK293T cells, and cell supernatants were collected after 48 and 72 hours. Functionality of bi-specific antibody constructs were tested using a co-culture of human healthy donor T cells and fluorescently labelled U87vIII target cells. Addition of HEK293 supernatants containing bi-specific antibody targeting an irrelevant antigen (CD22) and CD3, or an sdAb021-CD3 bi-specific antibody lacking a hinge domain did not result in significant killing of target cells; whereas addition of an sdAb021-CD3 bi-specific antibody containing a CD8-hinge element resulted in clear increases in T cell proliferation and tumour cell death (FIG. 30). FIG. 31 depicts relative growth of U87-vIII target cells in T-cell/tumour cell co-cultures with or without addition of cell supernatants containing various bi-specific T cell engager molecules. Healthy donor derived T cells and fluorescently labelled EGFR-negative Ramos cells or EGFR-expressing U87vIII target cells were placed in co-culture and examined using live fluorescence microscopy (Incucyte Device, Sartorius USA). The relative expansion of target cells was then assessed via automated cell counting. Control cells show growth repression with a CD22-targeted bi-specific T cell engager, but not EGFR-targeted engagers. In contrast, clear growth repression of U87vIII cells by T cells in the presence of EGFR-targeted bi-specific antibody molecules containing a full CD8-hinge domain (right) is observed. In summary, quantitation of tumour cell proliferation using automated cell counting of fluorescently labelled target cells reveals no significant impact on the growth of EGFR-negative target cells when either sdAb021 bi-specific antibody is included, but growth of EGFR-positive cells is specifically inhibited in T-cell cocultures containing long-hinge sdAb021-CD3 bi-specific antibody.

Discussion

Novel CAR constructs were sought that would be able to effectively target cells overexpressing EGFR and to discriminate between high level expression on tumours and lower expression on normal cells. Despite some variation in binding affinity for the three EGFR sdAb moieties tested here (Table 6), all three sdCAR constructs showed strong responses to EGFR-high SKOV3 and EGFR-low MCF7 target cells. Intriguingly, all EGFR-sdAb CAR receptors showed responsiveness to MCF7 cells despite no apparent reactivity of the purified sdAbs to EGFR-low MCF7 cells. These results are consistent with previous observations that EGFR specific CARs are relatively insensitive to ABD affinity up to the micromolar range[46] and underscore the exquisite antigen sensitivity of CAR-T cells to respond to and lyse even very low antigen expressing target cells[52]. This phenomenon possibly relates to the extreme multi-valency of both CAR and EGFR on their respective cells. Increased valency is well known to lead to significant avidity effects that boost the apparent affinity of biological interactions[53], and these would presumably apply to CAR-T cells as well.

While CAR constructs with maximal antigen sensitivity might be desirable in certain contexts, such as for CAR-T therapies targeting B cell family restricted antigens, the presence of EGFR expression on normal tissue requires a targeting strategy that would be selective for EGFR-overexpressing cancer cells. Previously, affinity modulation has been used to increase selectivity of CAR constructs for overexpressing cells[46]. While it is certainly possible to decrease sdAb affinity through various molecular strategies[54], mutational antibody changes can sometimes lead to unpredictable binding behaviour such as unexpected off-target binding, elevated tonic signaling, or loss of efficacy. Thus, an alternate strategy was pursued to decrease on target activity through hinge modification.

It is shown herein that even very small truncations of a human CD8 hinge, down to the level of individual amino acids, can be a powerful and remarkably precise tuning mechanism for CAR signaling. For the EGFR sdCAR tested most extensively, there was a sharp drop-off of CAR signaling over a range of only 10 amino acids within the CD8 hinge motif. While the requirements of lentiviral production and primary T cell transduction dictated that only a limited number of constructs in primary T cells could be tested, it would be intriguing to map the optimal hinge length for antigen induced CAR-T killing or CAR-T expansion in genuine donor-derived T cells. The hinge-truncation data using either membrane proximal (trastuzumab) or membrane-distal (anti-EGFRvIII-mAbs) based CAR constructs provides a clear demonstration of the criticality of epitope location as a determinant of hinge-sensitivity for CAR molecules reacting to tumour cells.

While the exact epitope for the EGFR sdAbs tested is not known, the data here would indicate a likely membrane proximal location. Consistent with this, sdAb028 cross-reacts with human and mouse but not cynomolgus EGFR; the only positions at which mouse and human EGFR sequences are identical but diverge from cynomolgus EGFR are in domain IV of EGFR. The finding that a full length CD8 hinge is required for the trastuzumab scFv-CAR seems to be consistent with previous experiments using similar CARs where hinge domains were also included[57], although it is worth noting that the scFv employed there diverges somewhat from that used here. In contrast to hinge truncation, it was found that a longer than necessary hinge does not have a very deleterious effect on CAR signaling, at least as determined by the CAR-J assay. Previous work has indicated that longer hinges can decrease in vivo activity for membrane-distal epitope targeting CARs[55], but follow-up studies pinpointed the effect to be related to FC-binding by IgG-hinge motifs rather than hinge length specifically[56]. For those membrane-proximal targeting CARs where a hinge is required though, the data demonstrates that CD8-hinge truncation is a powerful molecular strategy to reduce CAR-antigen sensitivity without the need to alter the ABD.

Due to the relatively more demanding technical requirements of testing CAR-T constructs in primary T cells only a limited number of CAR constructs was tested. Nonetheless, data presented here provide additional evidence that molecular optimization using transient CAR expression in Jurkat cells is predictive of signaling in stably transduced primary CAR-T cells, as has been previously reported[47]. The wider use of such optimization methodology could lead to improved ABD/hinge design for future CAR products. It may be possible for instance to design CARs with customized signaling for application in CD4, CD8, gamma-delta T cells, or NK cells.

The in vivo experiments presented here underline the fundamental trade-off between on-target, on-tumour activity and strategies that might reduce on-target off-tumour signaling. Achieving a level of signaling that is adequate for potent tumour killing yet also eliminates the risk of on-target toxicity may be difficult. Although in isolation, the SKOV3-selective truncated EGFR-sdAb CARs which were tested here had relatively little therapeutic effect in vivo it may be possible to combine such reduced signal hinge-truncated EGFR-sdCAR in a multi-antigen targeting CAR strategy that will ultimately more effectively recognize and lyse tumour cells in a highly selective fashion. Importantly, the aggressive xenograft models used here are imperfect and would likely not perfectly predict CAR-T signaling and activity in humans with slower growing and/or metastatic disease. The clinical use of CARs with varying hinge lengths could also present an intriguing alternative safety pathway for clinical trials of solid tumour targeting CARs through hinge length escalation.

In the preceding description, for purposes of explanation, numerous details are set forth in order to provide a thorough understanding of the embodiments. However, it will be apparent to one skilled in the art that these specific details are not required.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

### REFERENCES

The following publications are incorporated by reference herein.
1. Herbst, R. S. and Shin, D. M. (2002) Monoclonal antibodies to target epidermal growth factor receptor-positive tumors: a new paradigm for cancer therapy. Cancer 94, 1593-1611 https://doi.org/10.1002/cncr.10372
2. Jonker, D. J., O'Callaghan, C. J., Karapetis, C. S., Zalcberg, J. R., Tu, D., Au, H.-J. et al. (2007) Cetuximab for the treatment of colorectal cancer. N. Engl. J. Med. 357, 2040-2048 https://doi.org/10.1056/NEJMoa071834
3. Bonner, J. A., Harari, P. M., Giralt, J., Azarnia, N., Shin, D. M., Cohen, R. B. et al. (2006) Radiotherapy plus cetuximab for squamous-cell carcinoma of the head and neck. N. Engl. J. Med. 354, 567-578 https://doi.org/10.1056/NEJMoaO53422
4. Bell, A., Wang, Z. J., Arbabi-Ghahroudi, M., Chang, T. A., Durocher, Y., Trojahn, U. et al. (2010) Differential tumor-targeting abilities of three single-domain antibody formats. Cancer Lett. 289, 81-90 https://doi.org/10.1016/j.canlet.2009.08.003
5. Gottlin, E. B., Xiangrong, G., Pegram, C., Cannedy, A., Campa, M. J. and Patz, Jr, E. F. (2009) Isolation of novel EGFR-specific VHH domains. J. Biomol. Screen. 14, 77-85 https://doi.org/10.1177/1087057108327064
6 Roovers, R. C., Laeremans, T., Huang, L., De Taeye, S., Verkleij, A. J., Revets, H. et al. (2007) Efficient inhibition of EGFR signaling and of tumour growth by antagonistic anti-EFGR nanobodies. Cancer Immunol. Immunother. 56, 303-317 https://doi.org/10.1007/s00262-006-0180-4
7. Salema, V., Manas, C., Cerden, L., Pinero-Lambea, C., Marin, E., Roovers, R. C. et al. (2016) High affinity nanobodies against human epidermal growth factor receptor selected on cells by E. coli display. MAbs 8, 1286-1301 https://doi.org/10.1080/19420862.2016.1216742
8. Roovers, R. C., Vosjan, M. J., Laeremans, T., el Khoulati, R., de Bruin, R. C., Ferguson, K. M. et al. (2011) A biparatopic anti-EGFR nanobody efficiently inhibits solid tumour growth. Int. J. Cancer 129, 2013-2024 https://doi.org/10.1002/ijc.26145
9. Niu, G., Murad, Y. M., Gao, H., Hu, S., Guo, N., Jacobson, O. et al. (2012) Molecular targeting of CEACAM6 using antibody probes of different sizes. J. Control Release 161, 18-24 https://doi.org/10.1016/j.jconrel.2012.04.043
10. Babiuk, L. A., Pontarollo, R., Babiuk, S., Loehr, B. and van Drunen Littel-van den Hurk, S. (2003) Induction of immune responses by DNA vaccines in large animals. Vaccine 21, 649-658 https://doi.org/10.1016/S0264-410X(02)00574-1
11. Peyrassol, X., Laeremans, T., Gouwy, M., Lahura, V., Debulpaep, M., Van Damme, J. et al. (2016) Development by genetic immunization of monovalent antibodies (nanobodies) behaving as antagonists of the human ChemR23 receptor. J. Immunol. 196, 2893-2901 https://doi.org/10.4049/jimmunol. 1500888
12. Peyrassol, X., Laeremans, T., Lahura, V., Debulpaep, M., El Hassan, H., Steyaert, J. et al. (2018) Development by genetic immunization of monovalent antibodies against human vasoactive intestinal peptide receptor 1 (VPAC1), new innovative, and versatile tools to study VPAC1 receptor function. Front. Endocrinol. 9, 153 https://doi.org/10.3389/fendo.2018.00153
13 van der Woning, B., De Boeck, G., Blanchetot, C., Bobkov, V., Klarenbeek, A., Saunders, M. et al. (2016) DNA immunization combined with scFv phage display identifies antagonistic GCGR specific antibodies and reveals new epitopes on the small extracellular loops. MAbs 8, 1126-1135 https://doi.org/10.1080/19420862.2016.1189050
14. Koch-Nolte, F., Reyelt, J., Schössow, B., Schwarz, N., Scheuplein, F., Rothenburg, S. et al. (2007) Single domain antibodies from llama effectively and specifically block T cell ecto-ADP-ribosyltransferase ART2.2 in vivo. FASEB J. 21, 3490-3498 https://doi.org/10.1096/fj.07-8661com
15. Danquah, W., Meyer-Schwesinger, C., Rissiek, B., Pinto, C., Serracant-Prat, A., Amadi, M. et al. (2016) Nanobodies that block gating of the P2X7 ion channel ameliorate inflammation. Sci. Transl. Med. 8, 366ra162 https://doi.org/i0.1126/scitranslmed.aaf8463
16. Fumey, W., Koenigsdorf, J., Kunick, V., Menzel, S., Schutze, K., Unger, M. et al. (2017) Nanobodies effectively modulate the enzymatic activity of CD38 and allow specific imaging of CD38(+) tumors in mouse models in vivo. Sci. Rep. 7, 14289 https://doi.org/10.1038/s41598-017-14112-6
17. Durocher, Y., Perret, S. and Kamen, A. (2002) High-level and high-throughput recombinant protein production by transient transfection of suspension-growing human 293-EBNA1 cells. Nucleic Acids Res. 30, E9 https://doi.org/10.1093/nar/30.2.e9
18. Henry, K. A., Hussack, G., Collins, C., Zwaagstra, J. C., Tanha, J. and MacKenzie, C. R. (2016) Isolation of TGF-beta-neutralizing single-domain antibodies of predetermined epitope specificity using next-generation DNA sequencing. Protein Eng. Des. Sel. 29, 439-443 https://doi.org/i0.1093/protein/gzw043
19. Rossotti, M. A., Pirez, M., Gonzelez-Techera, A., Cui, Y., Bever, C. S., Lee, K. S. et al. (2015) Method for sorting and pairwise selection of nanobodies for the development of highly sensitive sandwich immunoassays. Anal. Chem. 87, 11907-11914 https://doi.org/10.1021/acs.analchem.5b03561
20. Pardon, E., Laeremans, T., Triest, S., Rasmussen, S. G., Wohlkonig, A., Ruf, A. et al. (2014) A general protocol for the generation of nanobodies for structural biology. Nat. Protoc. 9, 674-693 https://doi.org/10.1038/nprot.2014.039
21. Hussack, G., Arbabi-Ghahroudi, M., van Faassen, H., Songer, J. G., Ng, K. K., MacKenzie, R. et al. (2011) Neutralization of Clostridium difficile toxin A with single-domain antibodies targeting the cell receptor binding domain. J. Biol. Chem. 286, 8961-8976 https://doi.org/10.1074/jbc.M110.198754
22. June C H, O'Connor R S, Kawalekar O U, Ghassemi S, Milone M C. CAR T cell immunotherapy for human cancer. Science. 2018; 359:1361-5.
23. Srivastava S, Riddell S R. Engineering CAR-T Cells: Design Concepts. Trends Immunol. 2015; 36:494-502.
24. Guedan S, Calderon H, Posey A D, Maus M V. Engineering and Design of Chimeric Antigen Receptors. Mol Ther Methods Clin Dev. 2018; 12:145-56.
25. Wong W Y, Tanha J, Krishnan L, Tian B, Kumar P, Gaspar K, et al. Abstract A74: CAR-T cells harboring camelid single domain antibody as targeting agent to CEACAM6 antigen in pancreatic cancer. Cancer Immunol Res. American Association for Cancer Research; 2017; 5:A74-A74.
26. Zhao W-H, Liu J, Wang B-Y, Chen Y-X, Cao X-M, Yang Y, et al. A phase 1, open-label study of LCAR-B38M, a chimeric antigen receptor T cell therapy directed against B cell maturation antigen, in patients with relapsed or refractory multiple myeloma. J Hematol OncolJ Hematol Oncol. 2018; 11:141.
27. Ghorashian S, Kramer A M, Onuoha S, Wright G, Bartram J, Richardson R, et al. Enhanced CAR T cell expansion and prolonged persistence in pediatric patients with ALL treated with a low-affinity CD19 CAR. Nat Med. 2019; 25:1408-14.
28. Alabanza L, Pegues M, Geldres C, Shi V, Wiltzius J J W, Sievers S A, et al. Function of Novel Anti-CD19 Chimeric Antigen Receptors with Human Variable Regions Is Affected by Hinge and Transmembrane Domains. Mol Ther. 2017; 25:2452-65.
29. Schafer D, Henze J, Pfeifer R, Schleicher A, Brauner J, Mockel-Tenbrinck N, et al. A Novel Siglec-4 Derived Spacer Improves the Functionality of CAR T Cells Against Membrane-Proximal Epitopes. Front Immunol. 2020; 11:1704.
30. Guest R D, Hawkins R E, Kirillova N, Cheadle E J, Arnold J, O'Neill A, et al. The role of extracellular spacer regions in the optimal design of chimeric immune receptors: evaluation of four different scFvs and antigens. J Immunother Hagerstown Md 1997. 2005; 28:203-11.
31. Feucht J, Sun J, Eyquem J, Ho Y-J, Zhao Z, Leibold J, et al. Calibration of CAR activation potential directs alternative T cell fates and therapeutic potency. Nat Med. 2019; 25:82-8.
32. Thomas R, Weihua Z. Rethink of EGFR in Cancer With Its Kinase Independent Function on Board. Front Oncol [Internet]. Frontiers; 2019 [cited 2020 Jul. 21]; 9. Available from: https://www.frontiersin.org/articles/10.3389/fonc.2019.00800/full #B1
33. Santarius T, Shipley J, Brewer D, Stratton M R, Cooper C S. A census of amplified and overexpressed human cancer genes. Nat Rev Cancer. Nature Publishing Group; 2010; 10:59-64.
34. Ferguson K M. A structure-based view of Epidermal Growth Factor Receptor regulation. Annu Rev Biophys. 2008; 37:353-73.
35. Garcia-Foncillas J, Sunakawa Y, Aderka D, Wainberg Z, Ronga P, Witzler P, et al. Distinguishing Features of Cetuximab and Panitumumab in Colorectal Cancer and Other Solid Tumors. Front Oncol [Internet]. Frontiers; 2019 [cited 2020 Jul. 21]; 9. Available from: https://www.frontiersin.org/articles/10.3389/fonc.2019.00849/full
36. Maennling A E, Tur M K, Niebert M, Klockenbring T, Zeppernick F, Gattenlöhner S, et al. Molecular Targeting Therapy against EGFR Family in Breast Cancer: Progress and Future Potentials. Cancers. Multidisciplinary Digital Publishing Institute; 2019; 11:1826.
37. Martinez M, Moon E K. CAR T Cells for Solid Tumors: New Strategies for Finding, Infiltrating, and Surviving in the Tumor Microenvironment. Front Immunol [Internet]. 2019 [cited 2019 Sep. 26]; 10. Available from: https://www.frontiersin.org/articles/10.3389/fimmu.2019.00128/full
38. Li J, Li W, Huang K, Zhang Y, Kupfer G, Zhao Q. Chimeric antigen receptor T cell (CAR-T) immunotherapy for solid tumors: lessons learned and strategies for moving forward. J Hematol OncolJ Hematol Oncol [Internet]. 2018 [cited 2020 Aug. 25]; 11. Available from: https://www.ncbi.nlm.nih.gov/pmc/articles/PMC5809840/
39. Morgan R A, Johnson L A, Davis J L, Zheng Z, Woolard K D, Reap E A, et al. Recognition of Glioma Stem Cells by Genetically Modified T Cells Targeting EGFRvIII and Development of Adoptive Cell Therapy for Glioma. Hum Gene Ther. 2012; 23:1043-53.
40. O'Rourke D M, Nasrallah M P, Desai A, Melenhorst J J, Mansfield K, Morrissette J J D, et al. A single dose of peripherally infused EGFRvII-directed CAR T cells mediates antigen loss and induces adaptive resistance in patients with recurrent glioblastoma. Sci Transl Med. 2017; 9:eaaa0984.
41. Feng K, Guo Y, Liu Y, Dai H, Wang Y, Lv H, et al. Cocktail treatment with EGFR-specific and CD133-specific chimeric antigen receptor-modified T cells in a patient with advanced cholangiocarcinoma. J Hematol OncolJ Hematol Oncol [Internet]. 2017 [cited 2020 Aug. 25]; 10. Available from: https://www.ncbi.nlm.nih.gov/pmc/articles/PMC5217546/
42. Feng K, Guo Y, Dai H, Wang Y, Li X, Jia H, et al. Chimeric antigen receptor-modified T cells for the immunotherapy of patients with EGFR-expressing advanced relapsed/refractory non-small cell lung cancer. Sci China Life Sci. 2016; 59:468-79.
43. Liu Y, Guo Y, Wu Z, Feng K, Tong C, Wang Y, et al. Anti-EGFR chimeric antigen receptor-modified T cells in metastatic pancreatic carcinoma: A phase I clinical trial. Cytotherapy [Internet]. 2020 [cited 2020 Aug. 25]; Available from: http://www.sciencedirect.com/science/article/pii/S1465324920306174
44. Guo Y, Feng K, Liu Y, Wu Z, Dai H, Yang Q, et al. Phase I Study of Chimeric Antigen Receptor-Modified T Cells in Patients with EGFR-Positive Advanced Biliary Tract Cancers. Clin Cancer Res. 2018; 24:1277-86.
45. Suurs F V, Lub-de Hooge M N, de Vries E G E, de Groot D J A. A review of bispecific antibodies and antibody constructs in oncology and clinical challenges. Pharmacol Ther. 2019; 201:103-19.
46. Liu X, Jiang S, Fang C, Yang S, Olalere D, Pequignot E C, et al. Affinity-tuned ErbB2 or EGFR chimeric antigen receptor T cells exhibit an increased therapeutic index against tumors in mice. Cancer Res. 2015; 75:3596-607.
47. Bloemberg D, Nguyen T, MacLean S, Zafer A, Gadoury C, Gurnani K, et al. A High-Throughput Method for Characterizing Novel Chimeric Antigen Receptors in Jurkat Cells. Mol Ther—Methods Clin Dev [Internet]. 2020 [cited 2020 Jan. 31]; Available from: http://www.sciencedirect.com/science/article/pii/S2329050120300231
48. Watanabe N, Bajgain P, Sukumaran S, Ansari S, Heslop H E, Rooney C M, et al. Fine-tuning the CAR spacer improves T-cell potency. OncoImmunology. 2016; 5:e1253656.
49. Moritz D, Groner B. A spacer region between the single chain antibody- and the CD3 zeta-chain domain of chimeric T cell receptor components is required for efficient ligand binding and signaling activity. Gene Ther. 1995; 2:539-46.
50. Qin L, Lai Y, Zhao R, Wei X, Weng J, Lai P, et al. Incorporation of a hinge domain improves the expansion of chimeric antigen receptor T cells. J Hematol OncolJ Hematol Oncol. 2017; 10:68.
51. Garrett J T, Rawale S, Allen S D, Phillips G, Forni G, Morris J C, et al. Novel Engineered Trastuzumab Conformational Epitopes Demonstrate In Vitro and In Vivo Antitumor Properties against HER-2/neu. J Immunol. American Association of Immunologists; 2007; 178:7120-31.
52. Stone J D, Aggen D H, Schietinger A, Schreiber H, Kranz D M. A sensitivity scale for targeting T cells with chimeric antigen receptors (CARs) and bispecific T-cell Engagers (BiTEs). Oncoimmunology. 2012; 1:863-73.
53. Vauquelin G, Charlton S J. Exploring avidity: understanding the potential gains in functional affinity and target residence time of bivalent and heterobivalent ligands. Br J Pharmacol. 2013; 168:1771-85.
54. Alonso-Camino V, Senchez-Martin D, Compte M, Nunez-Prado N, Diaz R M, Vile R, et al. CARbodies: Human Antibodies Against Cell Surface Tumor Antigens Selected From Repertoires Displayed on T Cell Chimeric Antigen Receptors. Mol Ther-Nucleic Acids [Internet]. 2013 [cited 2018 Aug. 30]; 2. Available from: https://www.cell.com/molecular-therapy-family/nucleic-acids/abstract/S2162-2531(16)30151-2
55. Hudecek M, Lupo-Stanghellini M-T, Kosasih P L, Sommermeyer D, Jensen M C, Rader C, et al. Receptor affinity and extracellular domain modifications affect tumor recognition by ROR1-specific chimeric antigen receptor T-cells. Clin Cancer Res Off J Am Assoc Cancer Res. 2013; 19:3153-64.
56. Hudecek M, Sommermeyer D, Kosasih P L, Silva-Benedict A, Liu L, Rader C, et al. The non-signaling extracellular spacer domain of chimeric antigen receptors is decisive for in vivo antitumor activity. Cancer Immunol Res. 2015; 3:125-35.
57. Zhao Y, Wang Q J, Yang S, Kochenderfer J N, Zheng Z, Zhong X, et al. A Herceptin-Based Chimeric Antigen Receptor with Modified Signaling Domains Leads to Enhanced Survival of Transduced T Lymphocytes and Antitumor Activity. J Immunol Baltim Md 1950. 2009; 183:5563-74.
58. Pohlmann P R, Mayer I A, Mernaugh R. Resistance to Trastuzumab in Breast Cancer. Clin Cancer Res Off J Am Assoc Cancer Res. 2009; 15:7479-91.

TABLE 7
Table of Sequences

| Seq. # | Description | | Sequence |
|---|---|---|---|
| 1 | NRC-sdAb021 | CDR1 | LSDFGSKH |
| 2 | NRC-sdAb021 | CDR2 | IFSSGAT |
| 3 | NRC-sdAb021 | CDR3 | NLVPPSPA |
| 4 | NRC-sdAb022 | CDR1 | GFAFERNG |
| 5 | NRC-sdAb022 | CDR2 | ISSGTT |
| 6 | NRC-sdAb022 | CDR3 | NLVPPTPY |
| 7 | NRC-sdAb023 | CDR1 | GIIFSRNG |
| 8 | NRC-sdAb023 | CDR2 | ITLSGTT |
| 9 | NRC-sdAb023 | CDR3 | NLVPPTSY |
| 10 | NRC-sdAb024 | CDR1 | GSSLYINA |
| 11 | NRC-sdAb024 | CDR2 | ISRGGDT |
| 12 | NRC-sdAb024 | CDR3 | NAVPPFPN |
| 13 | NRC-sdAb025 | CDR1 | GSSLTINA |
| 14 | NRC-sdAb025 | CDR2 | VYRGGGT |
| 15 | NRC-sdAb025 | CDR3 | NAVPPFPN |
| 16 | NRC-sdAb026 | CDR1 | GVIFGINT |
| 17 | NRC-sdAb026 | CDR2 | ISNAGST |
| 18 | NRC-sdAb026 | CDR3 | NYVPPSPA |
| 19 | NRC-sdAb027 | CDR1 | GFAFERNG |
| 20 | NRC-sdAb027 | CDR2 | ISSGTT |
| 21 | NRC-sdAb027 | CDR3 | NLIPPTPY |
| 22 | NRC-sdAb028 | CDR1 | GSFFSRDA |
| 23 | NRC-sdAb028 | CDR2 | ISSGGLS |
| 24 | NRC-sdAb028 | CDR3 | SIYEDGVWTS |
| 25 | NRC-sdAb032 | CDR1 | GSISSISV |
| 26 | NRC-sdAb032 | CDR2 | ITSGGIS |
| 27 | NRC-sdAb032 | CDR3 | YMYVHVGYWVDY |
| 28 | NRC-sdAb033 | CDR1 | ESIDSTYV |
| 29 | NRC-sdAb033 | CDR2 | LTPGGGS |
| 30 | NRC-sdAb033 | CDR3 | NAFHRLRGTHY |
| 31 | NRC-VH54 | CDR1 | GFTFQEAY |
| 32 | NRC-VH54 | CDR2 | IDSSVGST |
| 33 | NRC-VH54 | CDR3 | GTYDTEETTSSWEDEYQYAV |
| 34 | NRC-sdAb021 | Full length | QVQLVESGGGLVQPGGSLRLSCSVSLSDFGSKHLAWHRQA |
| | (CDRs | | PGKEREWVALIFSSGATNYANSVKGRFTISRDNSKNTVYL |
| | underlined) | | QMNSLKPEDTAVYYCNLVPPSPAWGQGTQVTVSS |
| 35 | NRC-sdAb022 | Full length | QVQLVESGGGLVQPGGSLKLSCAVSGFAFERNGVSWYRQA |
| | (CDRs | | PGKQREFVAVISSGTTTYKDSVKGRFTISRDNTRNTAYLQ |
| | underlined) | | MNNLKPEDTAVYYCNLVPPTPYWGQGTQVTVSS |
| 36 | NRC-sdAb023 | Full length | QVQLVESGGGLVQPGGSLRLSCTASGIIFSRNGVSWYRQA |
| | (CDRs | | PGKEREFVAVITLSGTTTYKDSVKGRFTISRDNTRNTGYL |
| | underlined) | | QMNNLKPEDTAVYYCNLVPPTSYWGQGTQVTVSS |
| 37 | NRC-sdAb024 | Full length | QVQLVESGGGLVQPGGSLRLSCAASGSSLYINAMTWYRQA |
| | (CDRs | | PGEQREFVAIISRGGDTKYSDSVKGRESISIDNAKNTVYL |
| | underlined) | | QMNSLKAEDTAVYYCNAVPPFPNWGQGTQVTVSS |
| 38 | NRC-sdAb025 | Full length | QVQLVESGGGLVQPGGSLRLSCAASGSSLTINAMTWYRQA |
| | (CDRs | | PGDEREFVAIVYRGGGTKYADSVKGRESISIDNAKNTVYL |
| | underlined) | | QMNSLKAEDTAVYYCNAVPPFPNWGQGTQVTVSS |
| 39 | NRC-sdAb026 | Full length | QVQLVESGGALVQPGGSLRLSCSASGVIFGINTLAWHRQA |
| | (CDRs | | PGKEREWVALISNAGSTKYAGSVKGRFTISRDNDKNTVYL |
| | underlined) | | QMDSLKPEDTAVYYCNYVPPSPAWGQGTQVTVSS |
| 40 | NRC-sdAb027 | Full length | QVKLEESGGGLVQPGGSLKLSCAVSGFAFERNGVSWYRQA |
| | (CDRs | | PGKQREFVAVISSGTTTYKDSVKGRFTISRDNTRNTAYLQ |
| | underlined) | | MNNLKPEDTAVYYCNLIPPTPYWGQGTQVTVSS |
| 41 | NRC-sdAb028 | Full length | EVOLVESGGGLVQPGGSLRLSCTVSGSFFSRDAMGWHRQA |
| | (CDRs | | PGKQREMVAGISSGGLSNHAESVKGRFTISRDNSKNTVYL |
| | underlined) | | QMNNLKPEDTAVYYCSIYEDGVWTSWGQGTQVTVSS |
| 42 | NRC-sdAb028-H1 | Full length | QVQLVESGGGVVQPGRSLRLSCTVSGSFFSRDAMGWHRQA |
| | (CDRs | | PGKGLEMVAGISSGGLSNHAESVKGRFTISRDNSKNTLYL |
| | underlined) | | QMNSLRAEDTAVYYCSIYEDGVWTSWGQGTLVTVSS |
| 43 | NRC-sdAb032 | Full length | QVKLEESGGGLVQPGGSLRLSCSASGSISSISVMGWYRQA |
| | (CDRs | | PGKORELVARITSGGISNYVDSVKGRFTISGDSAKNIVYL |
| | underlined) | | QMNSLKPEDTAVYYCYMYVHVGYWVDYWGQGTQVTVSS |
| 44 | NRC-sdAb032-H1 | Full length | QVQLVESGGGLVQPGRSLRLSCSASGSISSISVMGWYRQA |
| | (CDRs | | PGKGLELVARITSGGISNYVDSVKGRFTISRDSNSKNTLY |
| | underlined) | | LQMNSLRAEDTAVYYCYMYVHVGYWVDYWGQGTLVTVSS |
| 45 | NRC-sdAb033 | Full length | QVQLVESGGGLVQPGGSLRLSCAASESIDSIYVMQWYRQA |
| | (CDRs | | PEKPRDVVATLTPGGGSYYPEAVLGRFTISRDDAKNTVYL |
| | underlined) | | QMNSLKPEDTAVYYCNAFHRLRGTHYWGQGTQVTVSS |
| 46 | NRC-sdAb033-H1 | Full length | QVQLVESGGGVVQPGRSLRLSCAASESIDSIYVMQWYRQA |
| | (CDRs | | PEKGLDVVATLTPGGGSYYPESVKGRFTISRDNSKNTLYL |
| | underlined) | | QMNSLRAEDTAVYYCNAFHRLRGTHYWGQGTLVTVSS |
| 47 | NRC-sdAb033-H2 | Full length | QVQLVESGGGVVQPGRSLRLSCAASESIDSIYVMQWYRQA |
| | (CDRs | | PGKGLEVVATLTPGGGSYYPDSVKGRFTISRDNSKNTLYL |
| | underlined) | | QMNSLRAEDTAVYYCNAFHRLRGTHYWGQGTLVTVSS |
| 48 | NRC-VH54 | Full length | QVQLQESGGGLVQPGGSLRLSCAASGFTFQEAYMHWVRQA |
| | (CDRs | | PGKGLEWVCAIDSSVGSTYYADSVKGRFTCSRDNSKNTLY |
| | underlined) | | LOMNSLRAEDTAVYYCGTYDTEETTSSWEDEYQYAVWGKG |
| | | | TTVTVSS |

| | | |
|---|---|---|
| 49 | Group A CDR1 Consensus | X1X2X3X4X5X6X7X8 wherein: |
| | | X1 is L or G, |
| | | X2 is S, F, I, V, or F, |
| | | X3 is D, A, I, or S, |
| | | X4 is F or L, |
| | | X5 is G, E, S, Y, or T, |
| | | X6 is S, R, or I, |
| | | X7 is K or N, and |
| | | X8 is H, G, A, or T |
| 50 | Group A CDR2 Consensus | X9X10X11X12GX13T wherein |
| | | X9 is I or V, |
| | | X10 is F, S, T, or Y, |
| | | X11 is S, L, R, or N, |
| | | X12 is S, G, A, or is absent, and |
| | | X13 is A, T, D, G, or S |
| 51 | Group A CDR3 Consensus | NX14X15PPX16X17X18 wherein: |
| | | X14 is L, Y, or A, |
| | | X15 is V or I, |
| | | X16 is S, T, or F, |
| | | X17 is P or S, and |
| | | X18 is A, Y, or N |
| 52 | CAR modular construct | ATGCTCAGGCTGCTCTTGGCTCTCAACTTATTCCCTTCAA |
| | (restriction sites bolded) | TTCAAGTAACAGGAGGGTCTTCG[antigen binding |
| | | domain]GGAAGACTTCCTTTGCGAGACGACGGTGGCGGG |
| | | GGATCAGGTGGTGGAGGTAGCGGGGGAGGGGGCTCAGGCG |
| | | GTACAACTACGCCTGCACCTCGCCCACCGACCCCAGCACC |
| | | AACCATCGCTTCACAGCCTTTGAGCCTGCGACCAGAGGCA |
| | | TGTCGCCCTGCTGCGGGCGGTGCCGTTCATACTCGCGGAC |
| | | TTGATTTTGCGTGTGACGTCGTCTCGCCTTCTAAGCCCTT |
| | | TTGGGTGCTGGTGGTGGTTGGTGGAGTCCTGGCTTGCTAT |
| | | AGCTTGCTAGTAACAGTGGCCTTTATTATTTTCTGGGTGA |
| | | GGAAACGGGGCAGAAAGAAACTCCTGTATATATTCAAACA |
| | | ACCATTTATGCGACCAGTACAAACTACTCAAGAGGAAGAT |
| | | GGCTGTAGCTGCCGATTTCCAGAAGAAGAAGAAGGAGGAT |
| | | GTGAACTGCTGAGAGTGAAGTTCAGCAGGAGCGCAGACGC |
| | | CCCCGCGTACCAGCAGGGCCAGAACCAGCTCTATAACGAG |
| | | CTCAATCTAGGACGAAGAGAGGAGTACGATGTTTTGGACA |
| | | AGCGACGTGGCCGGGACCCTGAGATGGGGGGAAAGCCGCA |
| | | GAGAAGGAAGAACCCTCAGGAAGGCCTGTACAATGAACTG |
| | | CAGAAAGATAAGATGGCGGAGGCCTACAGTGAGATTGGGA |
| | | TGAAAGGCGAGCGCCGGAGGGGCAAGGGGCACGATGGCCT |
| | | TTACCAGGGACTCAGTACAGCCACCAAGGACACCTACGAC |
| | | GCCCTTCACATGCAGGCCCTGCCCCCTCGC[P2A-GFP |
| | | marker] |
| 53 | Human CD28 Signal Peptide | MLRLLLALNLFPSIQVTG |
| 54 | Synthetic Flexible Linker | GGGGSGGGGSGGGGSGG |
| | Domain | |
| 55 | Human CD8 Hinge Domain | TTTPAPRPPTPAPTIASQPLSLRPEACRPAAGGAVHTRGL |
| | | DFACD |
| 56 | Truncated 34CD8h | APTIASQPLSLRPEACRPAAGGAVHTRGLDFACD |
| 57 | Truncated 22CD8h | PEACRPAAGGAVHTRGLDFACD |
| 58 | Human CD28 Transmembrane | PSKPFWVLVVVGGVLACYSLLVTVAFIIFWVR |
| | Domain | |
| 59 | Human 4-1BB Co-stimulatory | KRGRKKLLYIFKQPFMRPVQTTQEEDGCSCRFPEEEEGGC |
| | Domain | EL |
| 60 | Human CD3zeta Signaling | LRVKFSRSADAPAYQQGQNOLYNELNLGRREEYDVLDKRR |
| | Domain | GRDPEMGGKPQRRKNPQEGLYNELQKDKMAEAYSEIGMKG |
| | | ERRRGKGHDGLYQGLSTATKDTYDALHMQALPPR |
| 61 | FMC63 scFV | DYKDHDGDYKDHDIDYKDDDDKDIQMTOTTSSLSASLGDR |
| | | VTISCRASQDISKYLNWYQQKPDGTVKLLIYHTSRLHSGV |
| | | PSRFSGSGSGTDYSLTISNLEQEDIATYFCQQGNTLPYTF |
| | | GGGTKLEITGSTSGSGKPGSGEGSTKGEVKLQESGPGLVA |
| | | PSQSLSVTCTVSGVSLPDYGVSWIRQPPRKGLEWLGVIWG |
| | | SETTYYNSALKSRLTIIKDNSKSQVFLKMNSLQTDDTAIY |
| | | YCAKHYYYGGSYAMDYWGQGTSVTVSS |
| 62 | Short flexible linker amino acid | GGGGS |
| | sequence | |
| 63 | EGFR1 used in CAR constructs | QVQLVESGGGLVQPGGSLRLSCSVSLSDFGSKHLAWHRQA |
| | | PGKEREWVALIFSSGATNYANSVKGRFTISRDNSKNTVYL |
| | | QMNSLKPEDTAVYYCNLVPPSPAWGQGTQVTVSS |
| 64 | EGFR7 used in CAR constructs | QVKLEESGGGLVQPGGSLKLSCAVSGFAFERNGVSWYRQA |
| | | PGKQREFVAVISSGTTTYKDSVKGRFTISRDNTRNTAYLQ |
| | | MNNLKPEDTAVYYCNLIPPTPYWGQGTQVTVSS |
| 65 | EGFR10 used in CAR | EVOLVESGGGLVQPGGSLRLSCTVSGSFFSRDAMGWHRQA |
| | constructs | PGKQREMVAGISSGGLSNHAESVKGRFTISRDNSKNTVYL |
| | | QMNNLKPEDTAVYYCSIYEDGVWTSWGQGTQVTVSS |
| 66 | Exemplary CAR DNA Sequence | ATGCTCAGGCTGCTCTTGGCTCTCAACTTATTCCCTTCAA |
| | EGFR1-45CD8h-BBz with full | TTCAAGTAACAGGAGGGTCTTCGCAGGTACAGCTGGTGGA |
| | human CD8hinge | GTCTGGGGGAGGCTTGGTGCAGCCGGGGGGATCTCTGAGA |
| | | CTCTCCTGCTCAGTGTCACTAAGCGACTTCGGGAGCAAGC |
| | | ATTTGGCCTGGCACCGCCAGGCTCCAGGGAAGGAGCGCGA |
| | | GTGGGTCGCACTTATATTTAGTTCTGGTGCCACAAACTAT |
| | | GCAAACTCCGTGAAGGGCCGATTCACCATCTCCAGAGACA |
| | | ACAGCAAGAACACAGTGTATCTGCAAATGAACAGCCTGAA |
| | | ACCTGAGGACACGGCCGTCTATTACTGCAATTTGGTTCCG |
| | | CCTTCTCCAGCCTGGGGCCAGGGGACCCAGGTCACTGTCT |
| | | CCTCAGGAAGACTTCCTTTGACAACTACGCCTGCACCTCG |
| | | CCCACCGACCCCAGCACCAACCATCGCTTCACAGCCTTTG |
| | | AGCCTGCGACCAGAGGCATGTCGCCCTGCTGCGGGCGGTG |
| | | CCGTTCATACTCGCGGACTTGATTTTGCGTGTGACCCTTC |
| | | TAAGCCCTTTTGGGTGCTGGTGGTGGTTGGTGGAGTCCTG |
| | | GCTTGCTATAGCTTGCTAGTAACAGTGGCCTTTATTATTT |
| | | TCTGGGTGAGGAAACGGGGCAGAAAGAAACTCCTGTATAT |
| | | ATTCAAACAACCATTTATGCGACCAGTACAAACTACTCAA |
| | | GAGGAAGATGGCTGTAGCTGCCGATTTCCAGAAGAAGAAG |
| | | AAGGAGGATGTGAACTGCTGAGAGTGAAGTTCAGCAGGAG |
| | | CGCAGACGCCCCCGCGTACCAGCAGGGCCAGAACCAGCTC |
| | | TATAACGAGCTCAATCTAGGACGAAGAGAGGAGTACGATG |
| | | TTTTGGACAAGCGACGTGGCCGGGACCCTGAGATGGGGGG |
| | | AAAGCCGCAGAGAAGGAAGAACCCTCAGGAAGGCCTGTAC |
| | | AATGAACTGCAGAAAGATAAGATGGCGGAGGCCTACAGTG |
| | | AGATTGGGATGAAAGGCGAGCGCCGGAGGGGCAAGGGGCA |
| | | CGATGGCCTTTACCAGGGACTCAGTACAGCCACCAAGGAC |
| | | ACCTACGACGCCCTTCACATGCAGGCCCTGCCCCCTCGC |
| 67 | Exemplary CAR amino acid | MLRLLLALNLFPSIQVTGGSSQVQLVESGGGLVQPGGSLR |
| | sequence | LSCSVSLSDFGSKHLAWHRQAPGKEREWVALIFSSGATNY |
| | EGFR1-45CD8h-BBz with full | ANSVKGRFTISRDNSKNTVYLQMNSLKPEDTAVYYCNLVP |
| | human CD8hinge amino acid | PSPAWGQGTQVTVSSGRLPLTTTPAPRPPTPAPTIASQPL |
| | | SLRPEACRPAAGGAVHTRGLDFACDPSKPFWVLVVVGGVL |
| | | ACYSLLVTVAFIIFWVRKRGRKKLLYIFKQPFMRPVQTTQ |
| | | EEDGCSCRFPEEEEGGCELLRVKESRSADAPAYOQGQNOL |
| | | YNELNLGRREEYDVLDKRRGRDPEMGGKPQRRKNPQEGLY |
| | | NELQKDKMAEAYSEIGMKGERRRGKGHDGLYQGLSTATKD |
| | | TYDALHMQALPPR |
| 68 | Bi-specific T cell engager | ATGGAGTTTGGGCTGAGCTGGGTTTTCCTCGTTGCTCTTT |
| | modular construct (restriction | TTAGAGGTGTCCAGTGTACAGGAGGGTCTTCG[sdAb_ |
| | sites bolded) | sequence]GAAGACTTCCTTGGAGGAGGCGGAAGT |
| | | [anti-CD3_Mab_heavy_chain]GGCGGTGGTGGGT |
| | | CAGGCGGCGGTGGGAGCGGAGGAGGTGGAAGC[anti- |
| | | CD3_Mab_Light_chain]CACCACCACCACCACCACTA |
| | | G |
| 69 | Exemplary Bi-specific T cell | ATGGAGTTTGGGCTGAGCTGGGTTTTCCTCGTTGCTCTTT |
| | engager DNA Sequence | TTAGAGGTGTCCAGTGTACAGGACAGGTACAGGTACAGCT |
| | EGFR1-(G4S)3-45CD8h- | GGTGGAGTCTGGGGGAGGCTTGGTGCAGCCGGGGGGATCT |
| | CD3Mab | CTGAGACTCTCCTGCTCAGTGTCACTAAGCGACTTCGGGA |
| | Contains linker-extended human | GCAAGCATTTGGCCTGGCACCGCCAGGCTCCAGGGAAGGA |
| | CD8hinge domain | GCGCGAGTGGGTCGCACTTATATTTAGTTCTGGTGCCACA |
| | | AACTATGCAAACTCCGTGAAGGGCCGATTCACCATCTCCA |
| | | GAGACAACAGCAAGAACACAGTGTATCTGCAAATGAACAG |
| | | CCTGAAACCTGAGGACACGGCCGTCTATTACTGCAATTTG |
| | | GTTCCGCCTTCTCCAGCCTGGGGCCAGGGGACCCAGGTCA |
| | | CTGTCTCCTCAGGAAGACTTCCTTTGCGAGACGACGGTGG |
| | | CGGGGGATCAGGTGGTGGAGGTAGCGGGGGAGGGGGCTCA |
| | | GGCGGTACAACTACGCCTGCACCTCGCCCACCGACCCCAG |
| | | CACCAACCATCGCTTCACAGCCTTTGAGCCTGCGACCAGA |
| | | GGCATGTCGCCCTGCTGCGGGCGGTGCCGTTCATACTCGC |
| | | GGACTTGATTTTGCGTGTGACCTTGGAGGAGGCGGAAGT[ |
| | | human_CD3_specific_murine_IgG_Heavy_chain] |
| | | GGCGGTGGTGGGTCAGGCGGCGGTGGGAGCGGAGGAGG |
| | | TGGAAGC_[human_CD3_specific_murine_IgG |
| | | Light_chain] |
| 70 | Exemplary Bi-specific T cell | MEFGLSWVFLVALFRGVQCTGQVQLVESGGGLVQPGGSLR |
| | engager amino acid Sequence | LSCSVSLSDFGSKHLAWHRQAPGKEREWVALIFSSGATNY |
| | EGFR1-(G4S)3-45CD8h- | ANSVKGRFTISRDNSKNTVYLQMNSLKPEDTAVYYCNLVP |
| | CD3Mab | PSPAWGQGTQVTVSSGRLPLRDDGGGGSGGGGSGGGGSGG |
| | Contains linker-extended human | TTTPAPRPPTPAPTIASQPLSLRPEACRPAAGGAVHTRGL |
| | CD8hinge domain | DFACDLGGGGS[CD3_specific_Mab_heavy |
| | | chain]GGGGSGGGGSGGGGS |
| | | [CD3_specific_Mab_light_chain]HHHHHH |
| 71 | Untruncated Hinge | GGGGSGGGGSGGGGSGGTTTPAPRPPTPAPTIASQPLSLR |
| | (G₄S)₃GG + Human CD8 Hinge | PEACRPAAGGAVHTRGLDFACD |
| | Domain | |

1. (canceled)

2. An isolated single domain antibody (sdAb), which binds specifically to human epidermal growth factor receptor (EGFR), the sdAb comprising:
A)
i) a CDR1 amino acid sequence as set forth in SEQ ID NO: 1, a CDR2 amino acid sequence as set forth in SEQ ID NO: 2, and a CDR3 amino acid sequence as set forth in SEQ ID NO: 3 (from NRC-sdAb021),
ii) a CDR1 amino acid sequence as set forth in SEQ ID NO: 4, a CDR2 amino acid sequence as set forth in SEQ ID NO: 5, and a CDR3 amino acid sequence as set forth in SEQ ID NO: 6 (from NRC-sdAb022),
iii) a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9 (from NRC-sdAb023),
iv) a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12 (from NRC-sdAb024),
v) a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15 (from NRC-sdAb025),
vi) a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18 (from NRC-sdAb026),
vii) a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (from NRC-sdAb027),
viii) a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (from NRC-sdAb028),
ix) a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 (from NRC-sdAb032),
x) a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30 (from NRC-sdAb033)_or
xi) a CDR1 amino acid sequence as set forth in SEQ ID NO: 31, a CDR2 amino acid sequence as set forth in SEQ ID NO: 32, and a CDR3 amino acid sequence as set fort in SEQ ID NO: 33 (from NRC-VH54).

3-5. (canceled)

6. The isolated sdAb of claim 2, comprising A) the amino acid sequence of any one of SEQ ID NO: 34 to 48, or B) an amino acid sequence that is at least 80% identical to any one of SEQ ID NO: 34 to 48 across the full length thereof.

7. The isolated sdAb of claim 2, comprising A) the amino acid sequence of any one of SEQ ID NO: 34 to 48.

8-24. (canceled)

25. The isolated sdAb of claim 2, which is a camelid sdAb.

26. The isolated sdAb of claim 25, which is a llama sdAb.

27. The isolated sdAb of claim 2, which is a humanized sdAb of llama origin.

28. The isolated sdAb of claim 27, which comprises:
the amino acid sequence of SEQ ID NO: 42 (NRC-sdAb028-H1),
the amino acid sequence of SEQ ID NO: 44 (NRC-sdAb032-H1),
the amino acid sequence of SEQ ID NO: 46 (NRC-sdAb033-H1), or
the amino acid sequence of SEQ ID NO: 47 (NRC-sdAb033-H2).

29-30. (canceled)

31. A recombinant polypeptide comprising one or more sdAb as defined in claim 2.

32. A multivalent antibody comprising:
a first antigen-binding portion comprising the sdAb as defined in claim 2, and
a second antigen-binding portion.

33. The multivalent antibody of claim 32, wherein the second antigen-binding moiety binds specifically to a cell-surface marker of a T-cell.

34-56. (canceled)

57. The multivalent antibody of claim 32, wherein the sdAb comprises:
a CDR1 amino acid sequence as set forth in SEQ ID NO: 1,
a CDR2 amino acid sequence as set forth in SEQ ID NO: 2, and
a CDR3 amino acid sequence as set forth in SEQ ID NO: 3 (from sdAb021/EGFR1).

58. The multivalent antibody of claim 32, wherein the sdAb comprises SEQ ID NO: 34 (sdAb021/EGFR1).

59-60. (canceled)

61. A method of treating a cancer in subject comprising administering to the subject the multivalent antibody as defined in claim 32.

62. The method of claim 61, wherein the cancer comprises a solid tumour.

63. A chimeric antibody receptor (CAR), which binds to human EGFR, comprising the VHH sdAb as defined in claim 2.

64-79. (canceled)

80. The CAR of claim 63, wherein the sdAb comprises SEQ ID NO: 63 (sdAb021/EGFR1), SEQ ID NO: 64 (sdAb027/EGFR7), SEQ ID NO: 65 (sdAb028/EGFR10), or SEQ ID NO: 42 (sdAb028-H1).

81. The CAR of claim 63, where in the VHH sdAb comprises:
a CDR1 amino acid sequence as set forth in SEQ ID NO: 1,
a CDR2 amino acid sequence as set forth in SEQ ID NO: 2, and
a CDR3 amino acid sequence as set forth in SEQ ID NO: 3;
a CDR2 amino acid sequence as set forth in SEQ ID NO: 19,
a CDR2 amino acid sequence as set forth in SEQ ID NO: 20, and
a CDR3 amino acid sequence as set forth in SEQ ID NO: 21: or
a CDR2 amino acid sequence as set forth in SEQ ID NO: 22,
a CDR2 amino acid sequence as set forth in SEQ ID NO: 23, and
a CDR3 amino acid sequence as set forth in SEQ ID NO: 24.

82-83. (canceled)

84. A recombinant nucleic acid molecule encoding the sdAb as defined in claim 2.

85-87. (canceled)

88. An engineered cell expressing at the cell surface membrane the CAR as defined in claim 63.

89-90. (canceled)

91. A method of treating a cancer in a subject, comprising administering to the subject the engineered cell as defined in claim 88.

92. The use of claim 90 or the method of claim 91, wherein the cancer comprises a solid tumour.


SEQUENCE LISTING (71 sequences)

SEQ ID NO: 1 - protein, 8 aa, Artificial Sequence
  NRC-sdAb021 CDR1
LSDFGSKH

SEQ ID NO: 2 - protein, 7 aa, Artificial Sequence
  NRC-sdAb021 CDR2
IFSSGAT

SEQ ID NO: 3 - protein, 8 aa, Artificial Sequence
  NRC-sdAb021 CDR3
NLVPPSPA

SEQ ID NO: 4 - protein, 8 aa, Artificial Sequence
  NRC-sdAb022 CDR1
GFAFERNG

SEQ ID NO: 5 - protein, 6 aa, Artificial Sequence
  NRC-sdAb022 CDR2
ISSGTT

SEQ ID NO: 6 - protein, 8 aa, Artificial Sequence
  NRC-sdAb022 CDR3
NLVPPTPY

SEQ ID NO: 7 - protein, 8 aa, Artificial Sequence
  NRC-sdAb023 CDR1
GIIFSRNG

SEQ ID NO: 8 - protein, 7 aa, Artificial Sequence
  NRC-sdAb023 CDR2
ITLSGTT

SEQ ID NO: 9 - protein, 8 aa, Artificial Sequence
  NRC-sdAb023 CDR3
NLVPPTSY

SEQ ID NO: 10 - protein, 8 aa, Artificial Sequence
  NRC-sdAb024 CDR1
GSSLYINA

SEQ ID NO: 11 - protein, 7 aa, Artificial Sequence
  NRC-sdAb024 CDR2
ISRGGDT

SEQ ID NO: 12 - protein, 8 aa, Artificial Sequence
  NRC-sdAb024 CDR3
NAVPPFPN

SEQ ID NO: 13 - protein, 8 aa, Artificial Sequence
  NRC-sdAb025 CDR1
GSSLTINA

SEQ ID NO: 14 - protein, 7 aa, Artificial Sequence
  NRC-sdAb025 CDR2
VYRGGGT

SEQ ID NO: 15 - protein, 8 aa, Artificial Sequence
  NRC-sdAb025 CDR3
NAVPPFPN

SEQ ID NO: 16 - protein, 8 aa, Artificial Sequence
  NRC-sdAb026 CDR1
GVIFGINT

SEQ ID NO: 17 - protein, 7 aa, Artificial Sequence
  NRC-sdAb026 CDR2
ISNAGST

SEQ ID NO: 18 - protein, 8 aa, Artificial Sequence
  NRC-sdAb026 CDR3
NYVPPSPA

SEQ ID NO: 19 - protein, 8 aa, Artificial Sequence
  NRC-sdAb027 CDR1
GFAFERNG

SEQ ID NO: 20 - protein, 6 aa, Artificial Sequence
  NRC-sdAb027 CDR2
ISSGTT

SEQ ID NO: 21 - protein, 8 aa, Artificial Sequence
  NRC-sdAb027 CDR3
NLIPPTPY

SEQ ID NO: 22 - protein, 8 aa, Artificial Sequence
  NRC-scAb028 CDR1
GSFFSRDA

SEQ ID NO: 23 - protein, 7 aa, Artificial Sequence
  NRC-sdAb028 CDR2
ISSGGLS

SEQ ID NO: 24 - protein, 10 aa, Artificial Sequence
  NRC-sdAb028 CDR3
SIYEDGVWTS

SEQ ID NO: 25 - protein, 8 aa, Artificial Sequence
  NRC-sdAb032 CDR1
GSISSISV

SEQ ID NO: 26 - protein, 7 aa, Artificial Sequence
  NRC-sdAb032 CDR2
ITSGGIS

SEQ ID NO: 27 - protein, 12 aa, Artificial Sequence
  NRC-sdAb032 CDR3
YMYVHVGYWVDY

SEQ ID NO: 28 - protein, 8 aa, Artificial Sequence
  NRC-sdAb033 CDR1
ESIDSIYV

SEQ ID NO: 29 - protein, 7 aa, Artificial Sequence
  NRC-sdAb033 CDR2
LTPGGGS

SEQ ID NO: 30 - protein, 11 aa, Artificial Sequence
  NRC-sdAb033 CDR3
NAFHRLRGTHY

SEQ ID NO: 31 - protein, 8 aa, Artificial Sequence
  NRC-VH54 CDR1
GFTFQEAY

SEQ ID NO: 32 - protein, 8 aa, Artificial Sequence
  NRC-VH54 CDR2
IDSSVGST

SEQ ID NO: 33 - protein, 20 aa, Artificial Sequence
  NRC-VH54 CDR3
GTYDTEETTSSWEDEYQYAV

SEQ ID NO: 34 - protein, 114 aa, Artificial Sequence
  NRC-sdAb021
QVQLVESGGGLVQPGGSLRLSCSVSLSDFGSKHLAWHRQAPGKEREWVALIFSSGATNYANSVKGRFTISRDNSKNTVYLQMNSLKPEDTAVYYCNLVPPSPAWGQGTQVTVSS

SEQ ID NO: 35 - protein, 113 aa, Artificial Sequence
  NRC-sdAb022
QVQLVESGGGLVQPGGSLKLSCAVSGFAFERNGVSWYRQAPGKQREFVAVISSGTTTYKDSVKGRFTISRDNTRNTAYLQMNNLKPEDTAVYYCNLVPPTPYWGQGTQVTVSS

SEQ ID NO: 36 - protein, 114 aa, Artificial Sequence
  NRC-sdAb023
QVQLVESGGGLVQPGGSLRLSCTASGIIFSRNGVSWYRQAPGKEREFVAVITLSGTTTYKDSVKGRFTISRDNTRNTGYLQMNNLKPEDTAVYYCNLVPPTSYWGQGTQVTVSS

SEQ ID NO: 37 - protein, 114 aa, Artificial Sequence
  NRC-sdAb024
QVQLVESGGGLVQPGGSLRLSCAASGSSLYINAMTWYRQAPGEQREFVAIISRGGDTKYSDSVKGRFSISIDNAKNTVYLQMNSLKAEDTAVYYCNAVPPFPNWGQGTQVTVSS

SEQ ID NO: 38 - protein, 114 aa, Artificial Sequence
  NRC-sdAb025
QVQLVESGGGLVQPGGSLRLSCAASGSSLTINAMTWYRQAPGDEREFVAIVYRGGGTKYADSVKGRFSISIDNAKNTVYLQMNSLKAEDTAVYYCNAVPPFPNWGQGTQVTVSS

SEQ ID NO: 39 - protein, 114 aa, Artificial Sequence
  NRC-sdAb026
QVQLVESGGALVQPGGSLRLSCSASGVIFGINTLAWHRQAPGKEREWVALISNAGSTKYAGSVKGRFTISRDNDKNTVYLQMDSLKPEDTAVYYCNYVPPSPAWGQGTQVTVSS

SEQ ID NO: 40 - protein, 113 aa, Artificial Sequence
  NRC-sdAb027
QVKLEESGGGLVQPGGSLKLSCAVSGFAFERNGVSWYRQAPGKQREFVAVISSGTTTYKDSVKGRFTISRDNTRNTAYLQMNNLKPEDTAVYYCNLIPPTPYWGQGTQVTVSS

SEQ ID NO: 41 - protein, 116 aa, Artificial Sequence
  NRC-sdAb028
EVQLVESGGGLVQPGGSLRLSCTVSGSFFSRDAMGWHRQAPGKQREMVAGISSGGLSNHAESVKGRFTISRDNSKNTVYLQMNNLKPEDTAVYYCSIYEDGVWTSWGQGTQVTVSS

SEQ ID NO: 42 - protein, 116 aa, Artificial Sequence
  NRC-sdAb028-H1
QVQLVESGGGVVQPGRSLRLSCTVSGSFFSRDAMGWHRQAPGKGLEMVAGISSGGLSNHAESVKGRFTISRDNSKNTLYLQMNSLRAEDTAVYYCSIYEDGVWTSWGQGTLVTVSS

SEQ ID NO: 43 - protein, 118 aa, Artificial Sequence
  NRC-sdAb032
QVKLEESGGGLVQPGGSLRLSCSASGSISSISVMGWYRQAPGKQRELVARITSGGISNYVDSVKGRFTISGDSAKNIVYLQMNSLKPEDTAVYYCYMYVHVGYWVDYWGQGTQVTVSS

SEQ ID NO: 44 - protein, 119 aa, Artificial Sequence
  NRC-sdAb032-H1
QVQLVESGGGLVQPGRSLRLSCSASGSISSISVMGWYRQAPGKGLELVARITSGGISNYVDSVKGRFTISRDSNSKNTLYLQMNSLRAEDTAVYYCYMYVHVGYWVDYWGQGTLVTVSS

SEQ ID NO: 45 - protein, 117 aa, Artificial Sequence
  NRC-sdAb033
QVQLVESGGGLVQPGGSLRLSCAASESIDSIYVMQWYRQAPEKPRDVVATLTPGGGSYYPEAVLGRFTISRDDAKNTVYLQMNSLKPEDTAVYYCNAFHRLRGTHYWGQGTQVTVSS

SEQ ID NO: 46 - protein, 117 aa, Artificial Sequence
  NRC-sdAb033-H1
QVQLVESGGGVVQPGRSLRLSCAASESIDSIYVMQWYRQAPEKGLDVVATLTPGGGSYYPESVKGRFTISRDNSKNTLYLQMNSLRAEDTAVYYCNAFHRLRGTHYWGQGTLVTVSS

SEQ ID NO: 47 - protein, 117 aa, Artificial Sequence
  NRC-sdAb033-H2
QVQLVESGGGVVQPGRSLRLSCAASESIDSIYVMQWYRQAPGKGLEVVATLTPGGGSYYPDSVKGRFTISRDNSKNTLYLQMNSLRAEDTAVYYCNAFHRLRGTHYWGQGTLVTVSS

SEQ ID NO: 48 - protein, 127 aa, Artificial Sequence
  NRC-VH54
QVQLQESGGGLVQPGGSLRLSCAASGFTFQEAYMHWVRQAPGKGLEWVCAIDSSVGSTYYADSVKGRFTCSRDNSKNTLYLQMNSLRAEDTAVYYCGTYDTEETTSSWEDEYQYAVWGKGTTVTVSS

SEQ ID NO: 49 - protein, 8 aa, Artificial Sequence
  Group A CDR1 Consensus
  MISC_FEATURE (1)..(1): Xaa is L or G
  MISC_FEATURE (2)..(2): Xaa is S, F, I, V, or F
  MISC_FEATURE (3)..(3): Xaa is D, A, I, or S
  MISC_FEATURE (4)..(4): Xaa is F or L
  MISC_FEATURE (5)..(5): Xaa is G, E, S, Y, or T
  MISC_FEATURE (6)..(6): Xaa is S, R, or I
  MISC_FEATURE (7)..(7): Xaa is K or N
  MISC_FEATURE (8)..(8): Xaa is H, G, A, or T
XXXXXXXX

SEQ ID NO: 50 - protein, 7 aa, Artificial Sequence
  Group A CDR2 Consensus
  MISC_FEATURE (1)..(1): Xaa is I or V
  MISC_FEATURE (2)..(2): Xaa is F, S, T, or Y
  MISC_FEATURE (3)..(3): Xaa is S, L, R, or N
  MISC_FEATURE (4)..(4): Xaa is S, G, A, or is absent
  MISC_FEATURE (6)..(6): Xaa is A, T, D, G, or S
XXXXGXT

SEQ ID NO: 51 - protein, 8 aa, Artificial Sequence
  Group A CDR3 Consensus
  MISC_FEATURE (2)..(2): Xaa is L, Y, or A
  MISC_FEATURE (3)..(3): Xaa is V or I
  MISC_FEATURE (6)..(6): Xaa is S, T, or F
  MISC_FEATURE (7)..(7): Xaa is P or S
  MISC_FEATURE (8)..(8): Xaa is A, Y, or N
NXXPPXXX

SEQ ID NO: 52 - DNA, 846 bp, Artificial Sequence
  CAR Modular Construct
  misc_feature (63)..(64): positions separate by region encoding antigen binding domain
  misc_feature (846)..(846): position followed by PSA-GFP marker
atgctcaggctgctcttggctctcaacttattcccttcaattcaagtaacaggagggtcttcgggaagacttcctttgcgagacgacggtggcgggggatcaggtggtggaggtagcgggggagggggctcaggcggtacaactacgcctgcacctcgcccaccgaccccagcaccaaccatcgcttcacagcctttgagcctgcgaccagaggcatgtcgccctgctgcgggcggtgccgttcatactcgcggacttgattttgcgtgtgacgtcgtctcgccttctaagcccttttgggtgctggtggtggttggtggagtcctggcttgctatagcttgctagtaacagtggcctttattattttctgggtgaggaaacggggcagaaagaaactcctgtatatattcaaacaaccatttatgcgaccagtacaaactactcaagaggaagatggctgtagctgccgatttccagaagaagaagaaggaggatgtgaactgctgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 53 - protein, 18 aa, Artificial Sequence
  Human CD28 Signal Peptide
MLRLLLALNLFPSIQVTG

SEQ ID NO: 54 - protein, 17 aa, Artificial Sequence
  Synthetic Flexible Linker Domain
GGGGSGGGGSGGGGSGG

SEQ ID NO: 55 - protein, 45 aa, Artificial Sequence
  Human CD8 Hinge Domain
TTTPAPRPPTPAPTIASQPLSLRPEACRPAAGGAVHTRGLDFACD

SEQ ID NO: 56 - protein, 34 aa, Artificial Sequence
  Truncated 34CD8h
APTIASQPLSLRPEACRPAAGGAVHTRGLDFACD

SEQ ID NO: 57 - protein, 22 aa, Artificial Sequence
  Truncated 22CD8h
PEACRPAAGGAVHTRGLDFACD

SEQ ID NO: 58 - protein, 32 aa, Artificial Sequence
  Human CD28 Transmembrane Domain
PSKPFWVLVVVGGVLACYSLLVTVAFIIFWVR

SEQ ID NO: 59 - protein, 42 aa, Artificial Sequence
  Human 4-1BB Co-stimulatory Domain
KRGRKKLLYIFKQPFMRPVQTTQEEDGCSCRFPEEEEGGCEL

SEQ ID NO: 60 - protein, 114 aa, Artificial Sequence
  Human CD3zeta Signaling Domain
LRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 61 - protein, 267 aa, Artificial Sequence
  FMC63 scFV
DYKDHDGDYKDHDIDYKDDDDKDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 62 - protein, 5 aa, Artificial Sequence
  Short flexible linker amino acid sequence
GGGGS

SEQ ID NO: 63 - protein, 114 aa, Artificial Sequence; identical to SEQ ID NO: 34
  EGFR1 used in CAR constructs

SEQ ID NO: 64 - protein, 113 aa, Artificial Sequence; identical to SEQ ID NO: 40
  EGFR7 used in CAR constructs

SEQ ID NO: 65 - protein, 116 aa, Artificial Sequence; identical to SEQ ID NO: 41
  EGFR10 used in CAR constructs

SEQ ID NO: 66 - DNA, 1119 bp, Artificial Sequence
  EGFR1-45CD8h-BBz CAR with full CD8hinge
atgctcaggctgctcttggctctcaacttattcccttcaattcaagtaacaggagggtcttcgcaggtacagctggtggagtctgggggaggcttggtgcagccggggggatctctgagactctcctgctcagtgtcactaagcgacttcgggagcaagcatttggcctggcaccgccaggctccagggaaggagcgcgagtgggtcgcacttatatttagttctggtgccacaaactatgcaaactccgtgaagggccgattcaccatctccagagacaacagcaagaacacagtgtatctgcaaatgaacagcctgaaacctgaggacacggccgtctattactgcaatttggttccgccttctccagcctggggccaggggacccaggtcactgtctcctcaggaagacttcctttgacaactacgcctgcacctcgcccaccgaccccagcaccaaccatcgcttcacagcctttgagcctgcgaccagaggcatgtcgccctgctgcgggcggtgccgttcatactcgcggacttgattttgcgtgtgacccttctaagcccttttgggtgctggtggtggttggtggagtcctggcttgctatagcttgctagtaacagtggcctttattattttctgggtgaggaaacggggcagaaagaaactcctgtatatattcaaacaaccatttatgcgaccagtacaaactactcaagaggaagatggctgtagctgccgatttccagaagaagaagaaggaggatgtgaactgctgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 67 - protein, 373 aa, Artificial Sequence
  EGFR1-45CD8h-BBz CAR with full CD8hinge
MLRLLLALNLFPSIQVTGGSSQVQLVESGGGLVQPGGSLRLSCSVSLSDFGSKHLAWHRQAPGKEREWVALIFSSGATNYANSVKGRFTISRDNSKNTVYLQMNSLKPEDTAVYYCNLVPPSPAWGQGTQVTVSSGRLPLTTTPAPRPPTPAPTIASQPLSLRPEACRPAAGGAVHTRGLDFACDPSKPFWVLVVVGGVLACYSLLVTVAFIIFWVRKRGRKKLLYIFKQPFMRPVQTTQEEDGCSCRFPEEEEGGCELLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 68 - DNA, 165 bp, Artificial Sequence
  Bi-specific T cell Engager Modular Construct
  misc_feature (72)..(73): positions separated by region encoding sdAb
  misc_feature (99)..(100): positions separated by region enoding anti-CD3 Mab heavy chain
  misc_feature (144)..(145): positions separated by region encoding anti-CD3 Mab light chain
atggagtttgggctgagctgggttttcctcgttgctctttttagaggtgtccagtgtacaggagggtcttcggaagacttccttggaggaggcggaagtggcggtggtgggtcaggcggcggtgggagcggaggaggtggaagccaccaccaccaccaccactag

SEQ ID NO: 69 - DNA, 684 bp, Artificial Sequence
  EGFR1-(G4S)3-45CD8h-CD3Mab Bi-specific T cell Engager
  misc_feature (639)..(640): positions separated by anti-CD3 specific murine IgG heavy chain
  misc_feature (684)..(684): positions followed by positions separated by anti-CD3 specific murine IgG light chain
atggagtttgggctgagctgggttttcctcgttgctctttttagaggtgtccagtgtacaggacaggtacaggtacagctggtggagtctgggggaggcttggtgcagccggggggatctctgagactctcctgctcagtgtcactaagcgacttcgggagcaagcatttggcctggcaccgccaggctccagggaaggagcgcgagtgggtcgcacttatatttagttctggtgccacaaactatgcaaactccgtgaagggccgattcaccatctccagagacaacagcaagaacacagtgtatctgcaaatgaacagcctgaaacctgaggacacggccgtctattactgcaatttggttccgccttctccagcctggggccaggggacccaggtcactgtctcctcaggaagacttcctttgcgagacgacggtggcgggggatcaggtggtggaggtagcgggggagggggctcaggcggtacaactacgcctgcacctcgcccaccgaccccagcaccaaccatcgcttcacagcctttgagcctgcgaccagaggcatgtcgccctgctgcgggcggtgccgttcatactcgcggacttgattttgcgtgtgaccttggaggaggcggaagtggcggtggtgggtcaggcggcggtgggagcggaggaggtggaagc

SEQ ID NO: 70 - protein, 234 aa, Artificial Sequence
  EGFR1-(G4S)3-45CD8h-CD3Mab B-specific T cell Engager
  MISC_FEATURE (213)..(214): positions separated by CD3-specific Mab heavy chain
  MISC_FEATURE (228)..(288): positions followed by CD3-specific Mab light chain
MEFGLSWVFLVALFRGVQCTGQVQVQLVESGGGLVQPGGSLRLSCSVSLSDFGSKHLAWHRQAPGKEREWVALIFSSGATNYANSVKGRFTISRDNSKNTVYLQMNSLKPEDTAVYYCNLVPPSPAWGQGTQVTVSSGRLPLRDDGGGGSGGGGSGGGGSGGTTTPAPRPPTPAPTIASQPLSLRPEACRPAAGGAVHTRGLDFACDLGGGGSGGGGSGGGGSGGGGSHHHHHH

SEQ ID NO: 71 - protein, 62 aa, Artificial Sequence
  Untruncated Hinge
  MISC_FEATURE (1)..(17): (G4S)3GG
  MISC_FEATURE (18)..(62): Human CD8 Hinge Domain
GGGGSGGGGSGGGGSGGTTTPAPRPPTPAPTIASQPLSLRPEACRPAAGGAVHTRGLDFACD